(19) United States
(12) Patent Application Publication
(10) Pub. No.: US 2017/0260183 A1
(43) Pub. Date: Sep. 14, 2017

(54) BICYCLIC COMPOUNDS AS PEST CONTROL AGENTS

(71) Applicant: BAYER CROPSCIENCE AKTIENGESELLSCHAFT, Monheim am Rhein (DE)

(72) Inventors: Peter JESCHKE, Bergisch Gladbach (DE); Alexander ARLT, Köln (DE); Silvia CEREZO-GALVEZ, Langenfeld (DE); Arnd VOERSTE, Köln (DE); Martin FÜSSLEIN, Düsseldorf (DE); Reiner FISCHER, Monheim (DE); Thomas BRETSCHNEIDER, LOHMAR (DE); Kerstin ILG, Köln (DE); Olga MALSAM, Rösrath (DE); Peter LÖSEL, Leverkusen (DE)

(21) Appl. No.: 15/531,471

(22) PCT Filed: Nov. 30, 2015
(86) PCT No.: PCT/EP2015/078056

§ 371 Date: May 30, 2017

(30) Foreign Application Priority Data
Dec. 2, 2014 (EP) 14195948.6

(51) Int. Cl. C07D 471/04; A01N 43/56

(52) U.S. Cl. CPC C07D 471/04; A01N 43/56

(57) ABSTRACT

The present application relates to novel bicyclic compounds, to compositions comprising these compounds, to their use for controlling animal pests and to processes and intermediates for their preparation.

The present application relates to novel bicyclic compounds, to compositions comprising these compounds, to their use for controlling animal pests and to processes and intermediates for their preparation.

Recently, bicyclic compounds having insecticidal properties have been disclosed (WO 2015/038503 A1).

WO 2014/100695 A1 describes the preparation and pharmaceutical use of PRMTS inhibitors containing, inter alia, an N-substituted 2H-pyrazolo[3,4-b]pyridin-1-yl fragment. Furthermore, WO 2010/056999 describes the preparation of parasiticides containing an N-substituted 2H-pyrazolo[3,4-b]pyridin-1-yl fragment.

Modern crop protection compositions have to meet many demands, for example in relation to extent, persistence and spectrum of their action and possible use. Questions of toxicity and of combinability with other active ingredients or formulation auxiliaries play a role, as does the question of the cost and complexity involved in the synthesis of an active ingredient. In addition, resistances can occur. For all these reasons alone, the search for novel crop protection compositions cannot be considered complete, and there is a constant need for novel compounds having improved properties compared to the known compounds, at least in relation to individual aspects.

It was an object of the present invention to provide compounds which widen the spectrum of the pesticides under various aspects.

The object, and further objects which are not stated explicitly but can be discerned or derived from the connections discussed herein, are achieved by compounds of the formula (I)

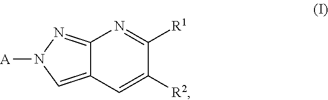

in which
A is a radical from the group of (A-a) to (A-f)

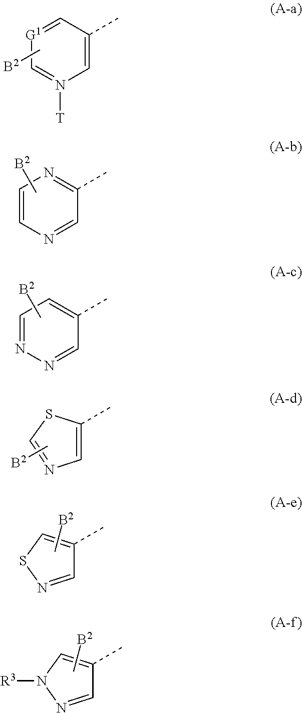

in which the broken line denotes the bond to the nitrogen atom of the bicyclic system of the formula (I) and
G¹ is N or C—B¹,
B¹ is a radical from the group of hydrogen, halogen, cyano, nitro, alkyl, haloalkyl, alkoxy, haloalkoxy and in each case optionally substituted cycloalkyl and cycloalkenyl,
B² is a radical from the group of hydrogen, halogen, cyano, nitro, alkyl, haloalkyl, alkoxy, haloalkoxy and in each case optionally substituted cycloalkyl and cycloalkenyl,
T is oxygen or an electron pair,
R¹ is a radical from the group of hydrogen, alkyl, alkoxy and cyano,
R² a) is a B radical from the group of (B-1) to (B-36)

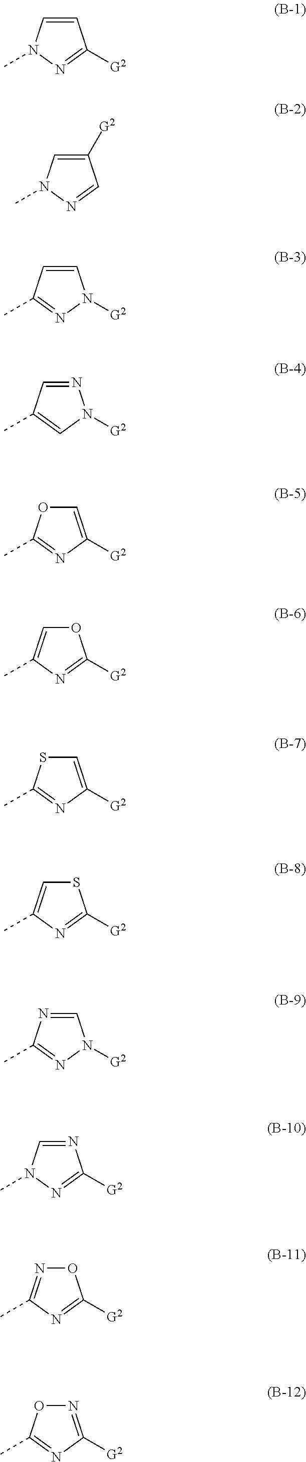

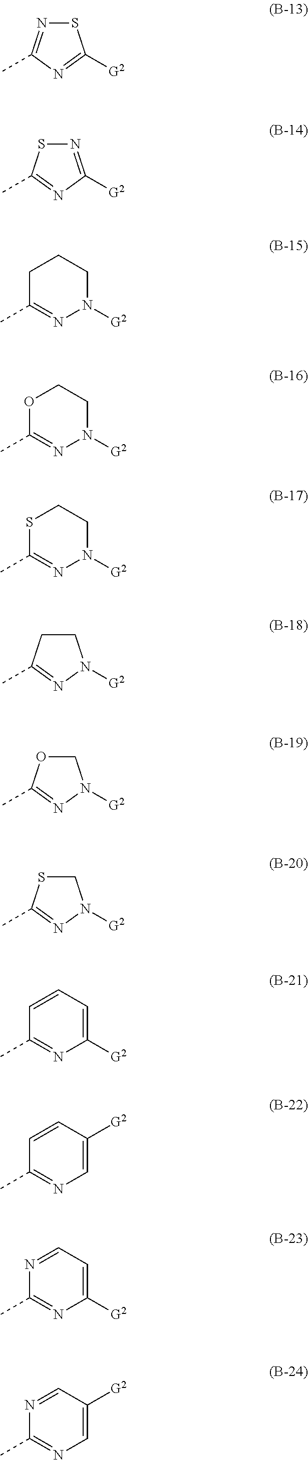

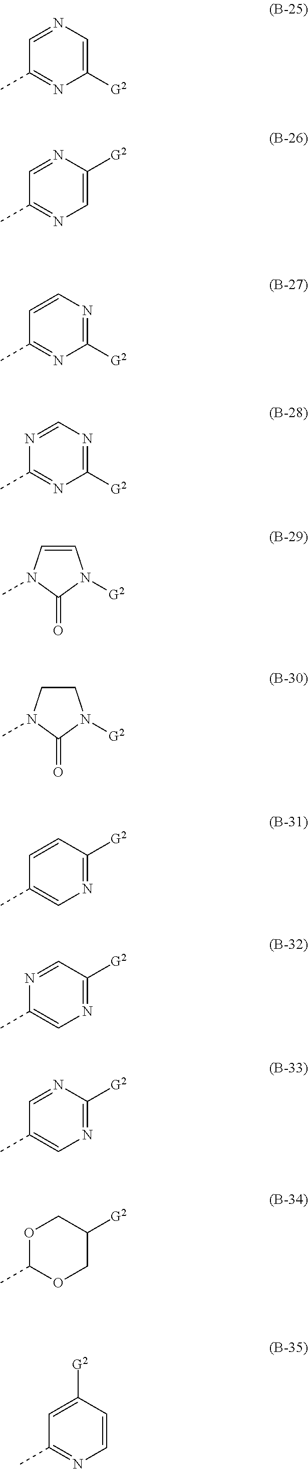

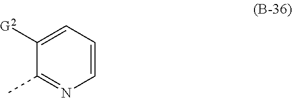

in which the broken line denotes the bond to the carbon atom of the bicyclic system of the formula (I), or
R² b) is a D radical from the group of (D-1) to (D-3)

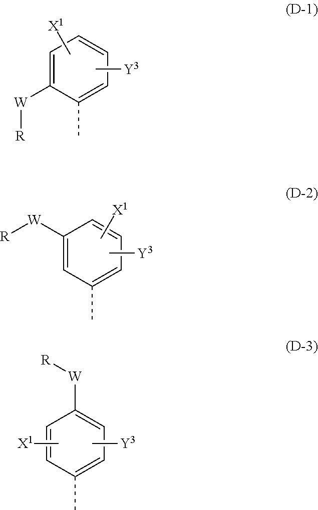

in which the broken line denotes the bond to the carbon atom of the bicyclic system of the formula (I), or
R² c) is a radical of the formula

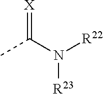

in which the broken line denotes the bond to the carbon atom of the bicyclic system of the formula (I), or
R² d) is a radical of the formula

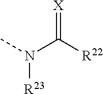

in which the broken line denotes the bond to the carbon atom of the bicyclic system of the formula (I), or
R² e) is an F radical from the group of (F-1) to (F-11)

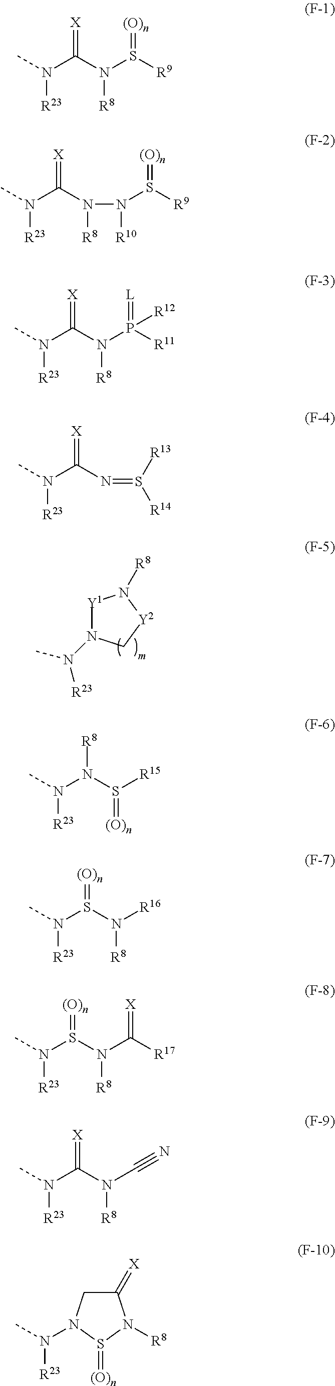

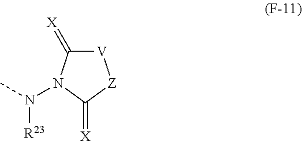

in which the broken line denotes the bond to the carbon atom of the bicyclic system of the formula (I), or
R² f) is a radical from the group of haloalkyl, carboxyl and amino,
   in which
G² is hydrogen or a radical from the group of halogen, nitro, amino, cyano, alkylamino, haloalkylamino, dialkylamino, alkyl, haloalkyl, alkoxycarbonylalkyl, saturated or unsaturated cycloalkyl which is optionally substituted and optionally interrupted by one or more heteroatoms, cycloalkylalkyl, alkoxy, haloalkoxy, alkoxyalkyl, halogenated alkoxyalkyl, alkylthioalkyl, alkylsulphinylalkyl, alkylsulphonylalkyl, bis(alkoxy)alkyl, bis(haloalkoxy) alkyl, alkoxy(alkylsulphanyl)alkyl, alkoxy(alkylsulphinyl)alkyl, alkoxy(alkylsulphonyl)alkyl, bis(alkylsulphanyl)alkyl, bis(haloalkylsulphanyl)alkyl, bis(hydroxyalkylsulphanyl)alkyl, alkoxy carbonyl, alkoxycarbonylalkyl, alpha-hydroxyiminoalkoxycarbonylalkyl, alpha-alkoxyiminoalkoxycarbonylalkyl, C(X²)NR³R⁴, NR⁶R⁷, alkylthio, alkylsulphinyl, alkylsulphonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may in turn be substituted by alkyl, haloalkyl, alkoxy and alkoxyalkyl), phenyl (which for its part may in turn be substituted by halogen, cyano, nitro, alkyl and haloalkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may in turn be substituted by halogen, nitro, alkyl, haloalkyl, alkoxy, haloalkoxy, alkoxyalkyl, alkylthio, alkylthioalkyl and cycloalkyl) and the heteroarylalkyl radicals triazolylalkyl, pyridylalkyl, pyrimidylalkyl and oxadiazolylalkyl (which for their part may in turn be substituted by halogen and alkyl), or
G² is a radical from the group of (C-1) to (C-9)

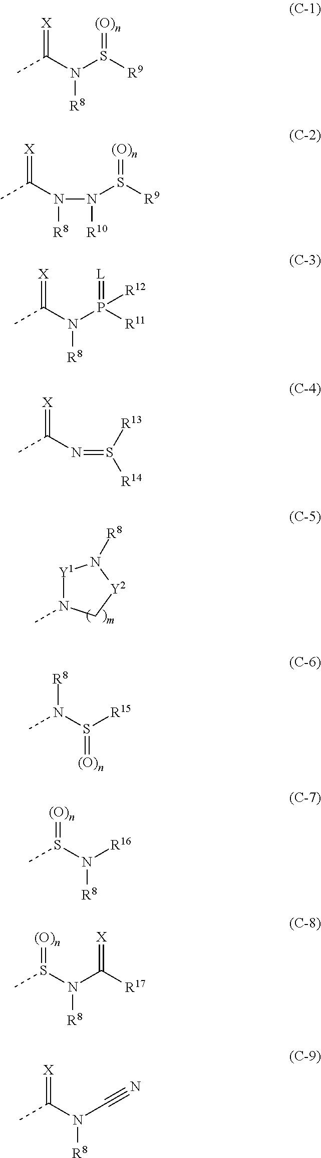

in which the broken line denotes the bond to the radicals (B-1) to (B-34),
X is oxygen or sulphur,
X¹ is a radical from the group of hydrogen, halogen, cyano, nitro, alkyl, haloalkyl, cycloalkyl, alkoxy and haloalkoxy,
X² is oxygen, sulphur, NR⁵ or NOH,
L is oxygen or sulphur,
V—Z is R²⁴CH—CHR²⁵ or R²⁴C═CR²⁵,
n is 1 or 2,
m is 1, 2, 3 or 4,
R is NR¹⁸R¹⁹, or is an in each case optionally substituted radical from the group of alkyl, alkenyl, alkynyl, alkoxyalkyl, alkyl-S-alkyl, alkyl-S(O)-alkyl, alkyl-S(O)₂-alkyl, R¹⁸—CO-alkyl, NR¹⁸R¹⁹—CO-alkyl, cycloalkyl, cycloalkenyl, cycloalkylalkyl, cycloalkenylalkyl, heterocyclyl, heterocyclylalkyl, phenyl, phenylalkyl, hetaryl and hetarylalkyl,
R³ is hydrogen or alkyl,
R⁴ is a radical from the group of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, alkylsulphinylalkyl, alkylsulphonylalkyl, aryl, arylalkyl and hetarylalkyl,
R⁵ is a radical from the group of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, aryl, arylalkyl and hetarylalkyl, or
R³ and R⁴ together with the nitrogen atom to which they are bonded form a ring which may contain one or more further heteroatoms from the group of nitrogen, oxygen and sulphur, or
R³ and R⁵ together with the nitrogen atoms to which they are bonded form a ring,
R⁶ is hydrogen or alkyl,
R⁷ is a radical from the group of hydrogen, alkyl, haloalkyl, cyanoalkyl, alkynyl, cycloalkyl, cycloalkylalkyl, alkoxy, haloalkoxy, alkoxyalkyl, alkylthioalkyl, alkylsulphinylalkyl, alkylsulphonylalkyl, alkoxycarbonyl, alkoxycarbonylalkyl, alkylthioalkyl, aryl, arylalkyl and hetarylalkyl, or
R⁶ and R⁷ together with the nitrogen atom to which they are bonded form a ring which may contain one or more further heteroatoms from the group of nitrogen, oxygen and sulphur,
R⁸ is a radical from the group of hydrogen, alkyl, haloalkyl, cyanoalkyl, alkoxy, haloalkoxy, alkenyl, alkoxyalkyl, in each case optionally halogen-substituted alkylcarbonyl and alkylsulphonyl, optionally halogen-substituted alkoxycarbonyl, optionally halogen-, alkyl-, alkoxy-, haloalkyl- and cyano-substituted cycloalkylcarbonyl, or is a cation, or an optionally alkyl- or arylalkyl-substituted ammonium ion,
R⁹ is a radical from the group of in each case optionally substituted alkyl, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R⁹ in the (C-1) and (F-1) radicals, together with the N—S(O)n group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁰ is hydrogen or alkyl,
R⁸ and R¹⁰ in the (C-2) and (F-2) radicals, together with the nitrogen atoms to which they are bonded, may also be a saturated or unsaturated and optionally substituted 4- to 8-membered ring which may contain at least one further heteroatom from the group of sulphur, oxygen (where oxygen atoms must not be directly adjacent to one another) and nitrogen and/or at least one carbonyl group,
R⁹ and R¹⁰ in the (C-2) and (F-2) radicals, together with the N—S(O)n group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹¹ is an in each case optionally substituted radical from the group of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹² is an in each case optionally substituted radical from the group of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹¹ and R¹² in the (C-3) and (F-3) radicals, together with the phosphorus atom to which they are bonded, may also form a saturated or unsaturated and optionally substituted 5- to 7-membered ring which may contain one or two heteroatoms from the group of oxygen (where oxygen atoms must not be directly adjacent to one another) and sulphur,
R¹³ is an in each case optionally substituted radical from the group of alkyl, alkenyl, alkynyl, phenyl and phenylalkyl,
R¹⁴ is an in each case optionally substituted radical from the group of alkyl, alkenyl, alkynyl, phenyl and phenylalkyl,
R¹⁵ is a radical from the group of in each case optionally substituted alkyl, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁵ in the (C-6) and (F-6) radicals, together with the N—S(O)n group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁶ is a radical from the group of hydrogen, in each case optionally substituted alkyl, alkoxy, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁶ in the (C-7) and (F-7) radicals, together with the nitrogen atom to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁷ is a radical from the group of in each case optionally substituted alkyl, alkoxy, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁷ in the (C-8) and (F-8) radicals, together with the N—C(X) group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁸ is a radical from the group of hydrogen, hydroxy, in each case optionally substituted alkyl, alkoxy, alkoxyalkyl, alkylthioalkyl, alkylsulphinylalkyl, alkylsulphonylalkyl, alkylcarbonyl, alkoxycarbonyl, alkenyl and alkynyl, in each case optionally substituted cycloalkyl, cycloalkylalkyl, cycloalkenyl and cycloalkenylalkyl, in which the rings may contain at least one heteroatom from the group of sulphur, oxygen (where oxygen atoms must not be directly adjacent to one another) and nitrogen, in each case optionally substituted aryl, arylalkyl, heteroaryl and heteroarylalkyl and an optionally substituted amino group,
R¹⁹ is a radical from the group of hydrogen, is an alkali metal or alkaline earth metal ion or is an ammonium ion optionally mono- to tetrasubstituted by C₁-C₄-alkyl or is an in each case optionally halogen- or cyano-substituted alkyl, alkoxy, alkoxyalkyl, alkylthioalkyl, alkylsulphinylalkyl, alkylsulphonylalkyl radical,
Y¹ and Y² are independently C═O or S(O)₂,
Y³ is a radical from the group of hydrogen, halogen, cyano, alkyl, cycloalkyl, haloalkyl, alkoxy, haloalkoxy and NR²⁰R²¹,
W is a radical from the group of O, S, SO and SO₂,
R²² is a radical from the group of alkyl, optionally halogen-, carbamoyl-, thiocarbamoyl- or cyano-substituted cycloalkyl, haloalkyl, alkoxy, haloalkoxy, alkoxyalkyloxy, alkylthio, alkylsulphinyl, alkylsulphonyl, haloalkylthio, haloalkylsulphinyl, haloalkylsulphonyl, alkylthioalkyl, alkylsulphinylalkyl, alkylsulphonylalkyl, alkylthioalkyloxy, alkylsulphinylalkyloxy, alkylsulphonylalkyloxy, haloalkylthioalkyl, haloalkylsulphinylalkyl, haloalkylsulphonylalkyl, alkylthioalkenyl, alkylsulphinylalkenyl, alkylsulphonylalkenyl, alkenylthioalkyl, alkenylsulphinylalkyl, alkenylsulphonylalkyl, alkylcarbonylalkyl, haloalkylcarbonylalkyl, alkoxyalkyl, haloalkoxy alkyl, alkoxycarbonylalkyl, haloalkoxycarbonylalkyl, alkylaminosulphonyl, di(alkylamino)sulphonyl, or
   in the case that R²=d)
R²² is also optionally substituted aryl or a radical from the group of E-1 to E-51

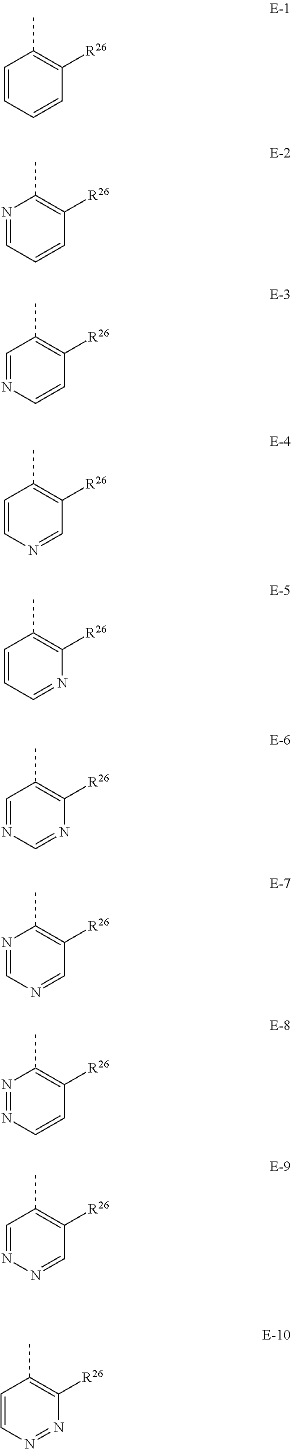

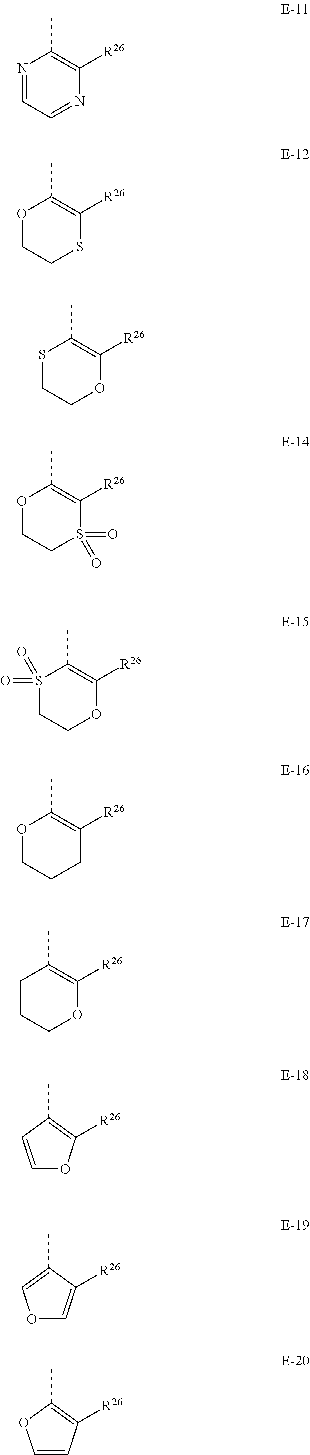

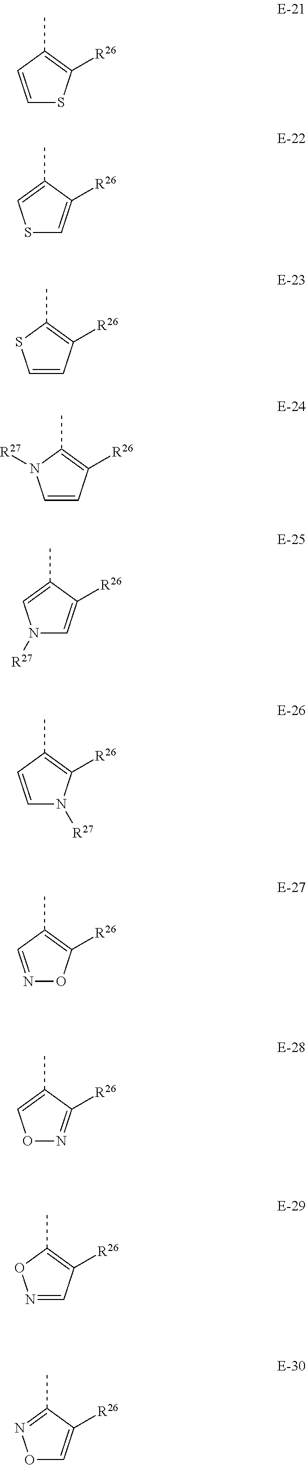

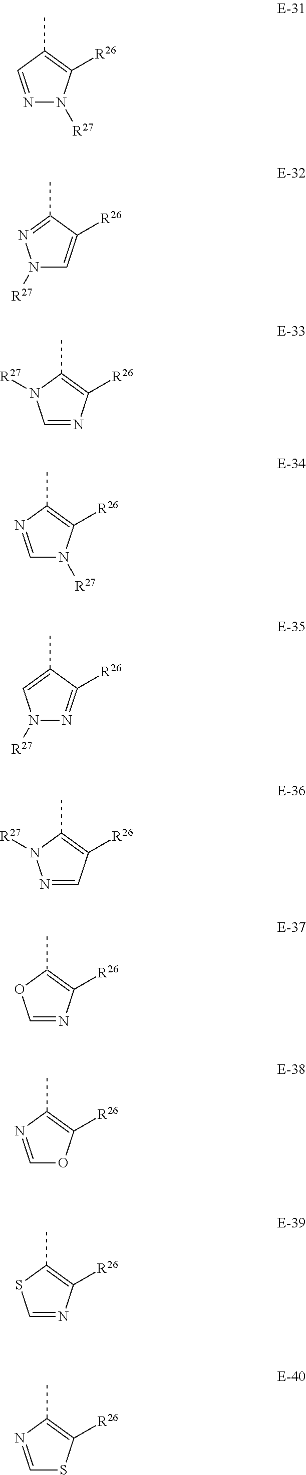

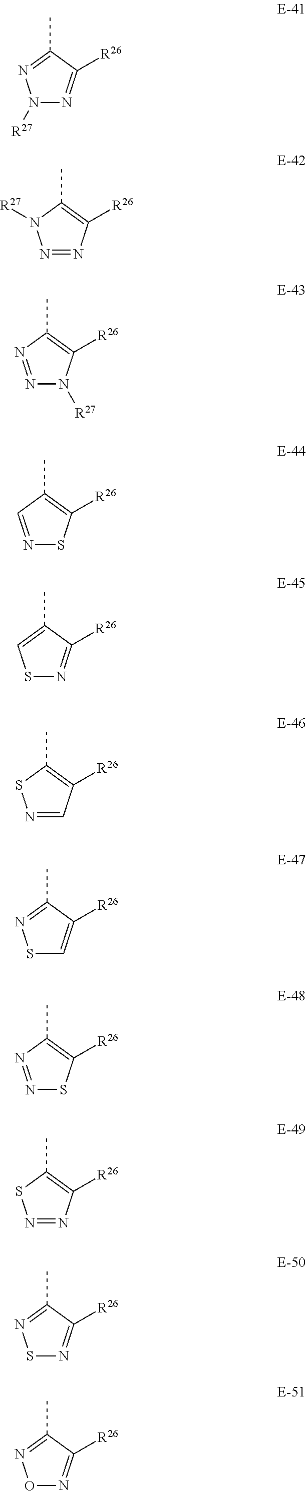

R²⁰ is a radical from the group of hydrogen, halogen, cyano, nitro, amino, hydroxy and in each case optionally substituted alkyl, alkenyl, alkynyl, cycloalkyl, cycloalkylalkyl, alkoxy, alkenyloxy, alkynyloxy, cycloalkyloxy, alkylcarbonyloxy, alkenylcarbonyloxy, alkynylcarbonyloxy, cycloalkylcarbonyloxy, alkoxycarbonyloxy, alkylsulphonyloxy, alkylamino, alkenylamino, alkynylamino, cycloalkylamino, alkylthio, haloalkylthio, alkenylthio, alkynylthio, cycloalkylthio, alkylsulphinyl, alkylsulphonyl, alkylcarbonyl, alkoxyiminoalkyl, alkoxycarbonyl, aminocarbonyl, alkylaminocarbonyl, dialkylaminocarbonyl, aminothiocarbonyl, alkylaminosulphonyl, alkylsulphonylamino, alkylcarbonylamino, alkenylcarbonylamino, alkynylcarbonylamino, cycloalkylcarbonylamino, alkoxycarbonylamino, alkylthiocarbonylamino, bicycloalkyl, aryl, aryloxy, heteroaryl and heteroaryloxy, where the substituents are independently of one another selected from halogen, cyano, nitro, hydroxy, amino, alkyl and haloalkyl,
R²¹ is a radical from the group of hydrogen, alkyl, cycloalkyl, haloalkyl, alkenyl, alkynyl, cycloalkylalkyl, cyanoalkyl, alkylcarbonyl, alkenylcarbonyl, haloalkylcarbonyl, haloalkenylcarbonyl, alkoxyalkyl, alkoxycarbonyl, alkylsulphonyl and haloalkylsulphonyl,
R²³ is a radical from the group of hydrogen, alkyl, alkenyl, alkynyl, cycloalkyl, cycloalkenyl, alkoxy, alkenyloxy, alkynyloxy, cycloalkyloxy, alkylthioalkyl, alkenylthioalkyl, cyanoalkyl, alkoxyalkyl and
R²⁴ is hydrogen or an in each case optionally substituted radical from the group of alkyl, alkenyl, alkynyl, phenyl and phenylalkyl and
R²⁵ is hydrogen or an in each case optionally substituted radical from the group of alkyl, alkenyl, alkynyl, phenyl and phenylalkyl,
R²⁷ is hydrogen or alkyl and
R²⁶ is a radical from the group of hydrogen, alkyl, haloalkyl, alkenyl, alkynyl, cycloalkyl, cycloalkylalkyl, alkoxyalkyl, alkylthioalkyl, alkylsulphinylalkyl, alkylsulphonylalkyl and cyanoalkyl.

It has been additionally found that the compounds of the formula (I) and also the compounds listed in Table 1 which are not covered by the formula (I) have good efficacy as pesticides, for example against arthropods and especially insects, and additionally generally have very good compatibility with plants, especially crop plants, and/or have favourable toxicological and/or favourable environmentally relevant properties.

Area of preference (1): Preference is given to compounds of the formula (I) in which

A is a radical from the group of (A-a), (A-b) and (A-f)

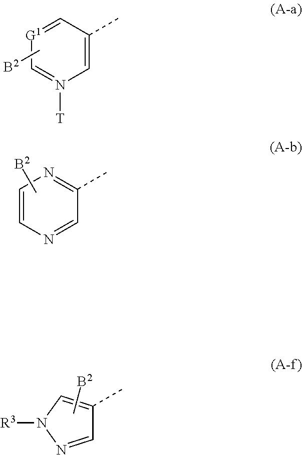

in which the broken line denotes the bond to the nitrogen atom of the bicyclic system and
G¹ is N or C—B¹,
B¹ is a radical from the group of hydrogen, halogen, C₁-C₆-alkyl and C₁-C₄-haloalkyl,
B² is a radical from the group of hydrogen, halogen, C₁-C₆-alkyl and C₁-C₄-haloalkyl,
T is oxygen or an electron pair,
R¹ is a radical from the group of hydrogen, C₁-C₆-alkyl and C₁-C₆-alkoxy,
R² a) is a radical from the group of (B-1) to (B-36)

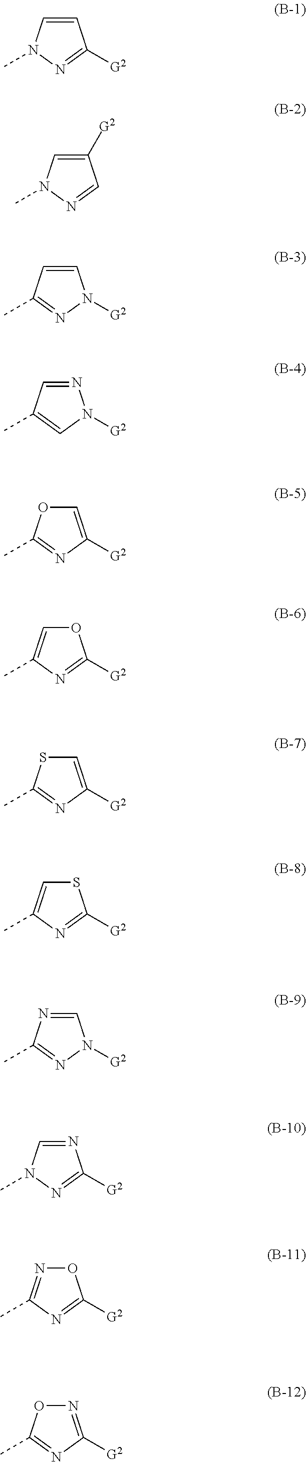

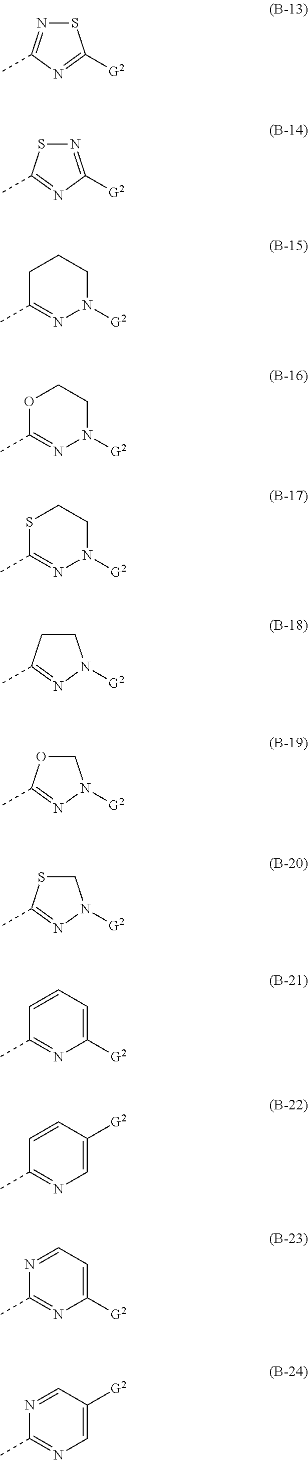

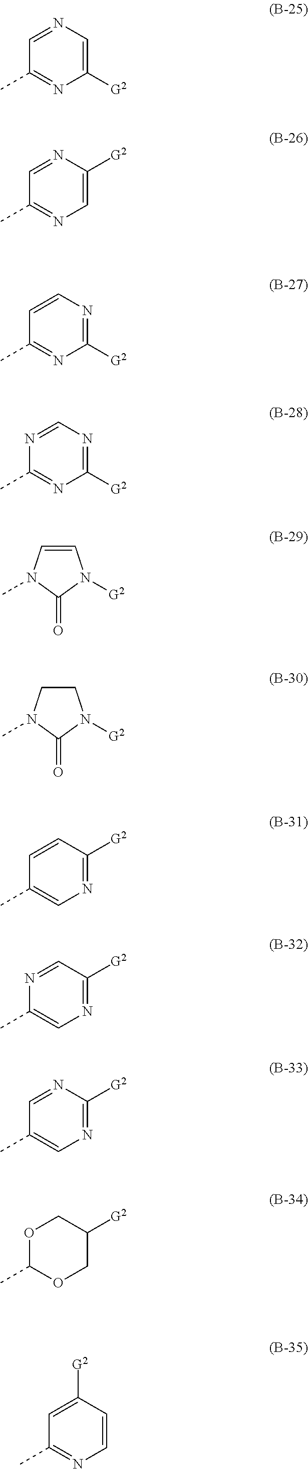

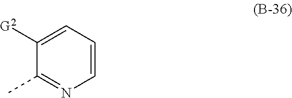

in which the broken line denotes the bond to the carbon atom of the bicyclic system or
R² b) is a radical from the group of (D-1) to (D-3)

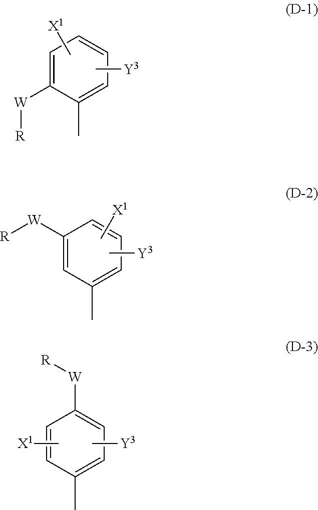

or
R² c) is a radical of the formula

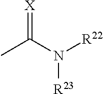

or
R² d) is a radical of the formula

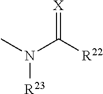

or
R² e) is a radical from the group of (F-1), (F-8), (F-10) and (F-11)

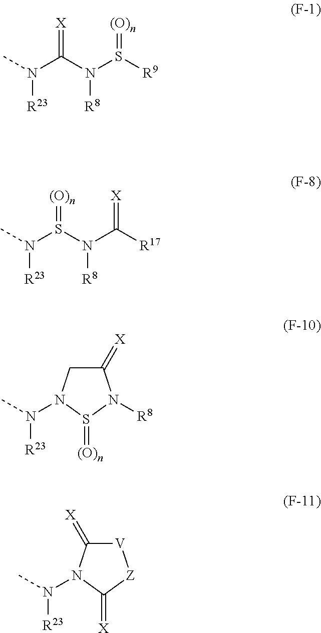

in which the broken line denotes the bond to the carbon atom in the formula (I) or
R² f) is a radical from the group of C₁-C₆-haloalkyl, carboxyl and amino,
   in which
G² is hydrogen or a radical from the group of halogen, nitro, amino, cyano, C₁-C₄-alkylamino, halo-C₁-C₄-alkylamino, C₁-C₄-dialkylamino, C₁-C₄-alkyl, halo-C₁-C₄-alkyl, C₁-C₄-alkoxy carbonyl-C₁-C₄-alkyl, C₃-C₆-cycloalkyl, C₃-C₆-cycloalkenyl, C₃-C₆-cycloalkyl-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy (C₁-C₄-alkylsulphanyl)-C₁-C₄-alkyl, C₁-C₄-alkoxy (C₁-C₄-alkylsulphinyl)-C₁-C₄-alkyl, C₁-C₄-alkoxy (C₁-C₄-alkylsulphonyl)-C₁-C₄-alkyl, bis(C₁-C₄-alkylsulphanyl)-C₁-C₄-alkyl, bis(halo-C₁-C₄-alkylsulphanyl)-C₁-C₄-alkyl, bis(hydroxy-C₁-C₄-alkylsulphanyl)-C₁-C₄-alkyl, C₁-C₄-alkoxy carbonyl, C₁-C₄-alkoxy carbonyl-C₁-C₄-alkyl, alpha-hydroxyimino-C₁-C₄-alkoxy carbonyl-C₁-C₄-alkyl, alpha-C₁-C₄-alkoxy-imino-C₁-C₄-alkoxycarbonyl-C₁-C₄-alkyl, C(X²)NR³R⁴, NR⁶1 e, C₁-C₄-alkylthio, C₁-C₄-alkylsulphinyl, C₁-C₄-alkylsulphonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may in turn be substituted by C₁-C₄-alkyl, halo-C₁-C₄-alkyl, C₁-C₄-alkoxy and C₁-C₄-alkoxy-C₁-C₄-alkyl), phenyl (which for its part may in turn be substituted by halogen, cyano, nitro, C₁-C₄-alkyl and halo-C₁-C₄-alkyl), the heteroaryl radicals pyridyl, pyridyl-N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may in turn be substituted by halogen, nitro, C₁-C₄-alkyl, halo-C₁-C₄-alkyl, C₁-C₄-alkoxy, halo-C₁-C₄-alkoxy, —C₄-alkoxy-C₄-alkyl, C₁-C₄-alkylthio, C₁-C₄-alkylthio-C₁-C₄-alkyl and C₃-C₆-cycloalkyl) and the heteroaryl-C₁-C₄-alkyl radicals triazolyl-C₁-C₄-alkyl, pyrimidyl-C₁-C₄-alkyl and oxadiazolyl-C₁-C₄-alkyl (which for their part may in turn be substituted by halogen and C₁-C₄-alkyl), or

G² is a radical from the group of (C-1) and (C-6) to (C-9)

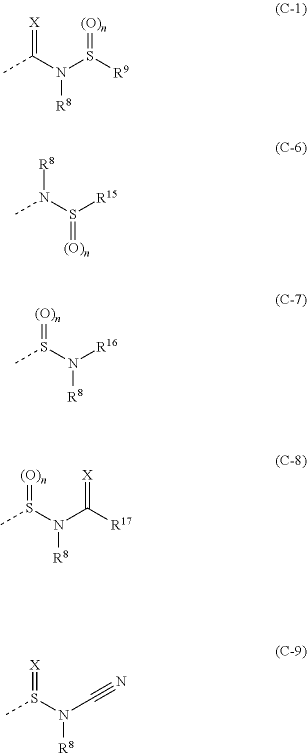

in which the broken line denotes the bond to the radicals (B-1) to (B-34),
X is oxygen or sulphur,
X¹ is a radical from the group of hydrogen, halogen, cyano, nitro, C₁-C₆-alkyl, C₁-C₆-haloalkyl, C₃-C₆-cycloalkyl, C₁-C₆-alkoxy and C₁-C₆-haloalkoxy,
X² is oxygen, sulphur, NR⁵ or NOH,
V—Z is R²⁴CH—CHR²⁵ or R²⁴C═CR²⁵,
n is 1 or 2,
R is NR¹⁸R¹⁹ or in each case optionally halogen-, oxygen- (leads to C═O) or cyano-substituted C₁-C₆-alkyl, C₃-C₆-alkenyl, C₃-C₆-alkynyl, C₁-C₆-alkoxy-C₁-C₄-alkyl, C₁-C₆-alkyl-S—C₁-C₄-alkyl, C₁-C₆-alkyl-S(O)₂—C₁-C₄-alkyl, is R¹⁸—CO—C₁-C₄-alkyl, is NR¹⁸R¹⁹—CO—C₁-C₄-alkyl, is optionally mono- or di-oxygen- (leads to C═O), —C₁-C₄-alkoxy- and —C₁-C₄-haloalkyl-substituted C₃-C₆-cycloalkyl, is optionally mono- or di-oxygen-(leads to C═O), —C₁-C₄-alkyl-, —C₃-C₆-cycloalkyl-, —C₄-alkoxy- and —C₁-C₄-haloalkyl-substituted C₃-C₈-cycloalkenyl, is optionally mono- or di-oxygen-(leads to C═O), C₃-C₆-cycloalkyl-, —C₁-C₄-alkoxy- and —C₁-C₄-haloalkyl-substituted is optionally mono- or di-oxygen-(leads to C═O), —C₁-C₄-alkyl, —C₃-C₆-cycloalkyl-, —C₁-C₄-alkoxy- and —C₁-C₄-haloalkyl-substituted C₃-C₆-cycloalkenyl-C₁-C₄-alkyl, is optionally mono- or di-oxygen-(leads to C═O), —C₁-C₄-alkyl-, —C₃-C₆-cycloalkyl-, —C₁-C₄-alkoxy- and —C₁-C₄-haloalkyl-substituted heterocyclyl, is optionally mono- or di-oxygen-(leads to C═O), —C₁-C₄-alkyl-, —C₃-C₆-cycloalkyl-, —C₁-C₄-alkoxy- and —C₁-C₄-haloalkyl-substituted heterocyclyl-C₁-C₄-alkyl or is in each case optionally mono- to tri-halogen-, -cyano-, —C₁-C₄-alkyl-, —C₁-C₄-haloalkyl-, —C₃-C₆-cycloalkyl-, —C₁-C₄-alkoxy- or —C₁-C₄-haloalkoxy-substituted phenyl, phenyl-C₁-C₄-alkyl, hetaryl and hetaryl-C₁-C₄-alkyl,
R³ is hydrogen or C₁-C₆-alkyl,
R⁴ is a radical from the group of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl,
R⁵ is a radical from the group of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl, or
R³ and R⁴ together with the nitrogen atom to which they are bonded form a 4- to 7-membered ring which may contain one or two further heteroatoms from the group of nitrogen, oxygen and sulphur (where oxygen and sulphur atoms must not be directly adjacent to one another),
R⁶ is hydrogen or C₁-C₄-alkyl,
R⁷ is a radical from the group of hydrogen, C₁-C₄-alkyl, halo-C₁-C₄-alkyl, cyano-C₁-C₄-alkyl, C₂-C₄-alkynyl, C₃-C₆-cycloalkyl, C₃-C₄-cycloalkyl-C₁-C₄-alkyl, C₁-C₄-alkoxy, halo-C₁-C₄-alkoxy, C₁-C₄-alkoxy-C₁-C₄-alkyl, C₁-C₄-alkoxy carbonyl, C₁-C₄-alkoxy carbonyl-C₁-C₄-alkyl, C₁-C₄-alkylthio-C₁-C₄-alkyl, aryl, aryl-C₁-C₄-alkyl and hetaryl-C₁-C₄-alkyl, or
R⁶ and R⁷ together with the nitrogen atom to which they are bonded form a 4- to 7-membered ring which may contain one or two further heteroatoms from the group of nitrogen, oxygen and sulphur (where oxygen and sulphur atoms must not be directly adjacent to one another),
R⁸ is a radical from the group of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulphonyl, optionally halogen-substituted C₁-C₆-alkoxycarbonyl, optionally halogen-, C₁-C₆-alkyl-, C₁-C₆-alkoxy-, C₁-C₆-haloalkyl- and cyano-substituted C₃-C₆-cycloalkylcarbonyl, or is a cation or an optionally C₁-C₆-alkyl- or aryl-C₁-C₆-alkyl-substituted ammonium ion,
R⁹ is a radical from the group of in each case optionally halogen-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl- and C₁-C₆-haloalkylsulphonyl-substituted C₁-C₆-alkyl, C₂-C₆-alkenyl and C₂-C₆-alkynyl, in each case optionally halogen-, C₁-C₆-alkyl-, C₁-C₆-haloalkyl-, C₁-C₆-alkoxy- or C₁-C₆-haloalkoxy-substituted C₃-C₆-cycloalkyl, C₃-C₆-cycloalkyl-C₁-C₆-alkyl and C₃-C₆-cycloalkenyl, in which one ring member may be replaced by a heteroatom from the group of sulphur, oxygen (where oxygen atoms must not be directly adjacent to one another) and nitrogen (and here are especially

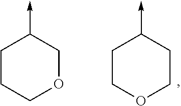

where the arrow in each case marks the bond to the sulphur atom in the (C-1) radical and in the (F-1) radical), in each case optionally halogen-, cyano- (including in the alkyl moiety), nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl-, C₁-C₆-haloalkylsulphonyl-, amino-, C₁-C₆-alkylamino-, C₁-C₆-alkylcarbonylamino-, C₁-C₆-alkoxycarbonylamino-, C₁-C₆-alkoxy-C₁-C₆-alkyl-, C₁-C₆-haloalkoxy-C₁-C₆-alkyl-, C₂-C₆-alkenyl-, C₂-C₆-alkynyl-, C₃-C₆-cycloalkyl-C₁-C₆-alkyl-, C₁-C₆-alkylcarbonyl-, C₁-C₆-alkoxycarbonyl- or aminocarbonyl-substituted aryl, heteroaryl, aryl-C₁-C₆-alkyl, heteroaryl-C₁-C₆-alkyl, or is NR′R″ in which R′ and R″ are independently a radical from the group of hydrogen, C₁-C₆-alkyl, C₁-C₆-haloalkyl, C₃-C₆-cycloalkyl, C₁-C₆-alkoxy, C₁-C₆-alkylcarbonyl and C₁-C₆-alkoxycarbonyl, or
R⁸ and R⁹ in the (C-1) radical and in the (F-1) radical, together with the N—S(O)n group to which they are bonded, may also form a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group of sulphur, oxygen (where oxygen atoms must not be directly adjacent to one another) and nitrogen and/or at least one and preferably one carbonyl group; in particular, R⁸ and R⁹ together with the N—S(O)n group to which they are bonded may be a radical from the group of

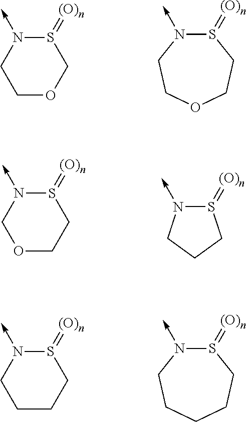

(in which the arrow in each case marks the bond to the C(X) group),
R¹⁵ is a radical from the group of in each case optionally methyl-, cyano-, carbamoyl-substituted C₁-C₆-alkyl, C₂-C₆-alkenyl and C₂-C₆-alkynyl, in each case optionally methyl-, trifluoromethyl-, halogen-, cyano- or carbamoyl-substituted C₃-C₆-cycloalkyl, C₃-C₆-cycloalkyl-C₁-C₂-alkyl and C₃-C₆-cycloalkenyl, in which the rings may contain at least one heteroatom from the group of sulphur, oxygen (where oxygen atoms must not be directly adjacent to one another) and nitrogen, in each case optionally C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-haloalkylsulphinyl-, C₁-C₄-haloalkylsulphonyl-, C₁-C₄-alkylamino-, di-(C₁-C₄-alkyl)-amino-, halogen-, nitro- or cyano-substituted aryl, heteroaryl, aryl-C₁-C₄-alkyl and heteroaryl-C₁-C₂-alkyl and an optionally C₁-C₄-alkyl-, C₁-C₄-alkylcarbonyl-, C₁-C₄-alkyl-C₁-C₄-alkoxycarbonyl- or C₁-C₄-alkylsulphonyl-substituted amino group,
R⁸ and R¹⁵ in the (C-6) radical, together with the N—S(O)n group to which they are bonded, may also form a saturated or unsaturated and optionally substituted 4- to 8-membered ring which may contain one or two further heteroatoms from the group of sulphur, oxygen (where oxygen and sulphur atoms must not be directly adjacent to one another) and nitrogen and/or at least one carbonyl group,
R¹⁶ is a radical from the group of hydrogen, in each case optionally methyl-, cyano-, carbamoyl- or carboxyl-substituted C₁-C₆-alkyl, C₁-C₆-alkoxy, C₂-C₄-alkenyl and C₂-C₄-alkynyl, in each case optionally halogen-, cyano-, nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl-, C₁-C₆-haloalkylsulphonyl-, amino-, C₁-C₆-alkylamino-, di-(C₁-C₆-alkyl)amino-, C₁-C₆-alkylcarbonylamino-, C₁-C₆-alkoxycarbonylamino-, C₂-C₆-alkenyl-, C₂-C₆-alkynyl- or C₁-C₆-alkylcarbonyl-substituted C₃-C₆-cycloalkyl, C₃-C₆-cycloalkyl-C₁-C₄-alkyl and C₃-C₆-cycloalkenyl in which the rings may contain at least one heteroatom from the group of sulphur, oxygen (where oxygen atoms must not be directly adjacent to one another) and nitrogen, in each case optionally halogen-, cyano-, nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl-, C₁-C₆-haloalkylsulphonyl-, amino-, C₁-C₆-alkylamino-, di-(C₁-C₆-alkyl)amino-, C₁-C₆-alkylcarbonylamino-, C₁-C₆-alkoxycarbonylamino-, C₂-C₆-alkenyl-, C₂-C₆-alkynyl- or C₁-C₆-alkylcarbonyl-substituted aryl, heteroaryl, aryl-C₁-C₂-alkyl and heteroaryl-C₁-C₂-alkyl and an optionally C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl-, C₁-C₆-haloalkylsulphonyl-, C₂-C₆-alkenyl-, C₂-C₆-alkynyl- or C₁-C₆-alkylcarbonyl-substituted amino group,
R¹⁷ is a radical from the group of in each case optionally halogen-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl- or C₁-C₆-haloalkylsulphonyl-substituted C₁-C₆-alkyl, C₁-C₆-alkoxy, C₂-C₆-alkenyl and C₂-C₆-alkynyl, in each case optionally halogen-, C₁-C₆-alkyl-, C₁-C₆-haloalkyl-, C₁-C₆-alkoxy- or C₁-C₆-haloalkoxy-substituted C₃-C₆-cycloalkyl, C₃-C₆-cycloalkyl-C₁-C₆-alkyl and C₃-C₆-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (including in the alkyl moiety), nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl-, C₁-C₆-haloalkylsulphonyl-, amino-, C₁-C₆-alkylamino-, di(C₁-C₆-alkyl)amino-, C₁-C₆-alkylcarbonylamino-, C₁-C₆-alkoxycarbonylamino-, C₁-C₆-alkoxy-C₁-C₆-alkyl-, C₁-C₆-haloalkoxy-C₁-C₆-alkyl-, C₂-C₆-alkenyl-, C₂-C₆-alkynyl-, C₃-C₆-cycloalkyl-C₁-C₆-alkyl-, C₁-C₆-alkylcarbonyl-, C₁-C₆-alkoxycarbonyl- or aminocarbonyl-substituted aryl, heteroaryl, aryl-C₁-C₆-alkyl, heteroaryl-C₁-C₆-alkyl or is NR′R″ in which R′ and R″ are each independently a radical from the group of hydrogen, C₁-C₆-alkyl, C₁-C₆-haloalkyl, C₃-C₆-cycloalkyl, C₁-C₆-alkoxy, C₁-C₆-alkylcarbonyl and C₁-C₆-alkoxycarbonyl,
R⁸ and R¹⁷ in the (C-8) radical and in the (F-8) radical, together with the N—C(X) group to which they are bonded, may also form a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two further heteroatoms from the group of sulphur, oxygen (where oxygen atoms must not be directly adjacent to one another) and nitrogen and/or one carbonyl group; in particular, R⁸ and R¹⁷ together with the N—C(X) group to which they are bonded may be a radical from the group of

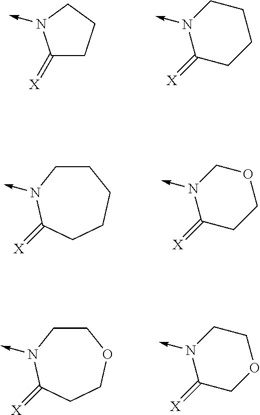

(where the arrow in each case denotes the bond to the sulphur atom in the (C-8) radical and in the (F-8) radical),
R¹⁸ is a radical from the group of hydrogen, hydroxyl, in each case optionally mono- or poly-halogen-substituted or mono- or di-cyano-substituted C₁-C₆-alkyl, C₁-C₆-alkoxy, C₁-C₆-alkoxy-C₁-C₄-alkyl, C₁-C₆-alkyl-S—C₁-C₄-alkyl, C₁-C₆-alkyl-S(O)—C₁-C₄-alkyl, C₁-C₆-alkyl-S(O)₂—C₁-C₄-alkyl, C₁-C₆-alkylcarbonyl, C₁-C₆-alkoxycarbonyl, C₃-C₆-cycloalkyl, C₃-C₆-cycloalkenyl, C₃-C₆-cycloalkyl-C₁-C₃-alkyl, C₃-C₆-cycloalkenyl-C₁-C₃-alkyl, heterocyclyl, heterocyclyl-C₁-C₃-alkyl and in each case optionally mono- to tetra-C₁-C₄-alkyl-, —C₁-C₄-haloalkyl-, —C₁-C₄-alkoxy-, —C₁-C₄-haloalkoxy-, —C₃-C₆-cycloalkyl-, -halogen- or -cyano-substituted phenyl, phenyl-C₁-C₃-alkyl, hetaryl and hetaryl-C₁-C₃-alkyl,
R¹⁹ is hydrogen, an alkali metal or alkaline earth metal ion or an optionally mono- to tetra-C₁-C₄-alkyl-substituted ammonium ion or an in each case optionally mono- or poly-halogen-substituted or mono- or di-cyano-substituted radical from the group of C₁-C₄-alkyl, C₁-C₄-alkoxy, C₁-C₄-alkoxy-C₁-C₄-alkyl, C₁-C₄-alkyl-S—C₁-C₄-alkyl, C₁-C₄-alkyl-S(O)—C₁-C₄-alkyl and C₁-C₄-alkyl-S(O)₂—C₁-C₄-alkyl,
Y³ is a radical from the group of hydrogen, halogen, cyano, C₁-C₆-alkyl, C₁-C₆-haloalkyl, C₃-C₆-cycloalkyl, C₁-C₆-alkoxy, C₁-C₆-haloalkoxy and NR²⁰R²¹,
W is a radical from the group of O, S, SO and SO₂,
R²² is a radical from the group of C₁-C₆-alkyl, optionally halogen-, carbamoyl-, thiocarbamoyl- or cyano-substituted C₃-C₆-cycloalkyl, C₁-C₆-haloalkyl, C₁-C₆-alkoxy, C₁-C₄-haloalkoxy, C₁-C₆-alkoxy-C₁-C₆-alkyloxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylthio-C₁-C₄-alkyl, C₁-C₄-alkylsulphinyl-C₁-C₄-alkyl, C₁-C₄-alkylsulphonyl-C₁-C₄-alkyl, C₁-C₄-alkylthio-C₁-C₄-alkyloxy, C₁-C₄-alkylsulphinyl-C₁-C₄-alkyloxy, C₁-C₄-alkylsulphonyl-C₁-C₄-alkyloxy, C₁-C₄-haloalkylthio-C₁-C₄-alkyl, C₁-C₄-haloalkylsulphinyl-C₁-C₄-alkyl, C₁-C₄-haloalkylsulphonyl-C₁-C₄-alkyl, C₁-C₄-alkylthio-C₂-C₄-alkenyl, C₁-C₄-alkylsulphinyl-C₂-C₄-alkenyl, C₁-C₄-alkylsulphonyl-C₂-C₄-alkenyl, C₂-C₄-alkenylthio-C₁-C₄-alkyl, C₂-C₄-alkenylsulphinyl-C₁-C₄-alkyl, C₂-C₄-alkenylsulphonyl-C₁-C₄-alkyl, C₁-C₄-alkylcarbonyl-C₁-C₄-alkyl, C₁-C₄-haloalkylcarbonyl-C₁-C₄-alkyl, C₁-C₄-alkoxy-C₁-C₄-alkyl, C₁-C₄-haloalkoxy-C₁-C₄-alkyl, C₁-C₄-alkoxycarbonyl-C₁-C₄-alkyl, C₁-C₄-haloalkoxycarbonyl-C₁-C₄-alkyl, C₁-C₄-alkylaminosulphonyl, di(C₁-C₄-alkyl)aminosulphonyl,
   in the case that R²=d)
R²² is also optionally halogen-, cyano-, nitro-, amino-, hydroxy-, C₁-C₆-alkyl-, C₁-C₆-haloalkyl-, C₂-C₆-alkenyl-, C₂-C₆-alkynyl-, C₃-C₆-cycloalkyl-, C₃-C₆-alkenyloxy-, C₃-C₆-alkynyloxy-, C₁-C₆-alkoxy-, C₁-C₄-haloalkoxy-, C₁-C₆-alkoxycarbonyloxy-, C₁-C₆-alkylamino-, C₃-C₆-alkenylamino-, C₃-C₆-alkynylamino-, C₃-C₆-cycloalkylamino-, C₁-C₆-alkylthio-, C₁-C₄-alkylsulphinyl-, C₁-C₄-alkylsulphonyl-, C₃-C₆-alkenylthio-, C₃-C₆-alkynylthio-, C₃-C₆-cycloalkylthio-, C₁-C₄-haloalkylthio-, C₁-C₄-haloalkylsulphinyl-, C₁-C₄-haloalkylsulphonyl-, C₁-C₆-alkylcarbonyl-, aminocarbonyl-, C₁-C₆-alkylaminocarbonyl-, di-(C₁-C₆)-alkylaminocarbonyl-, C₁-C₆-alkylcarbonylamino-, C₁-C₆-alkylamino- or C₁-C₆-dialkylamino-substituted aryl or is a radical from the group of E-1 to E-51

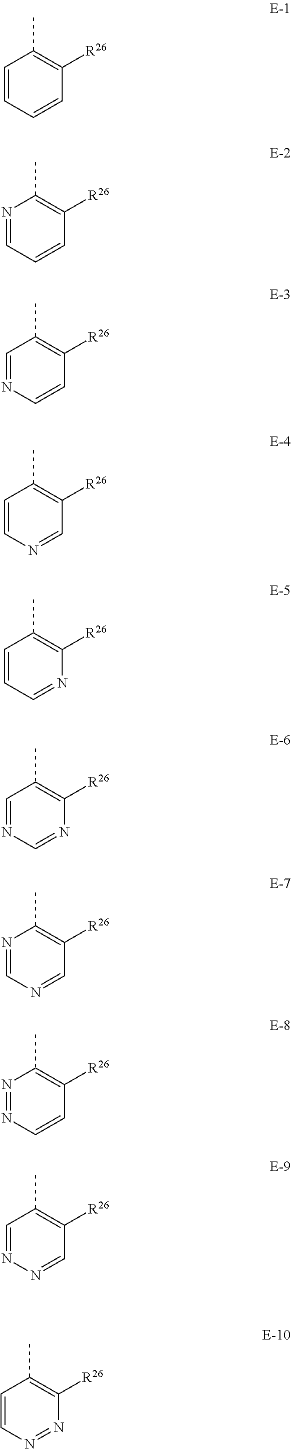

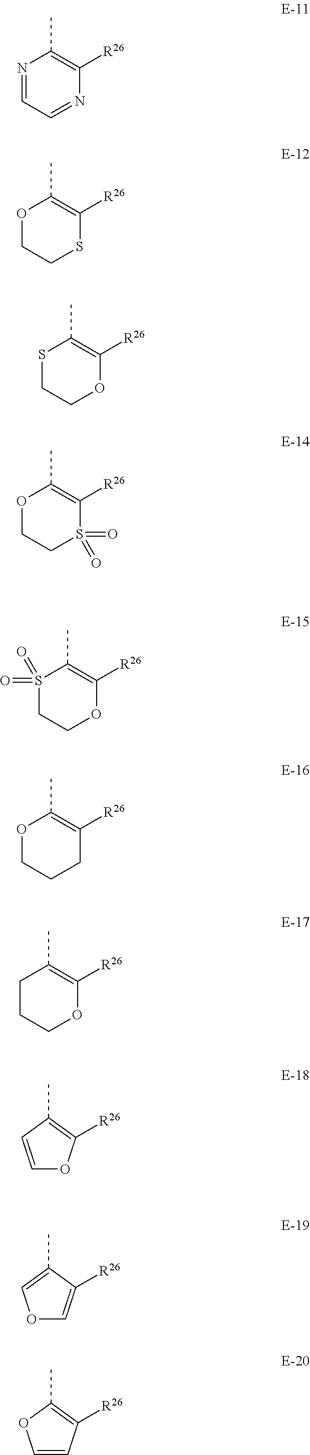

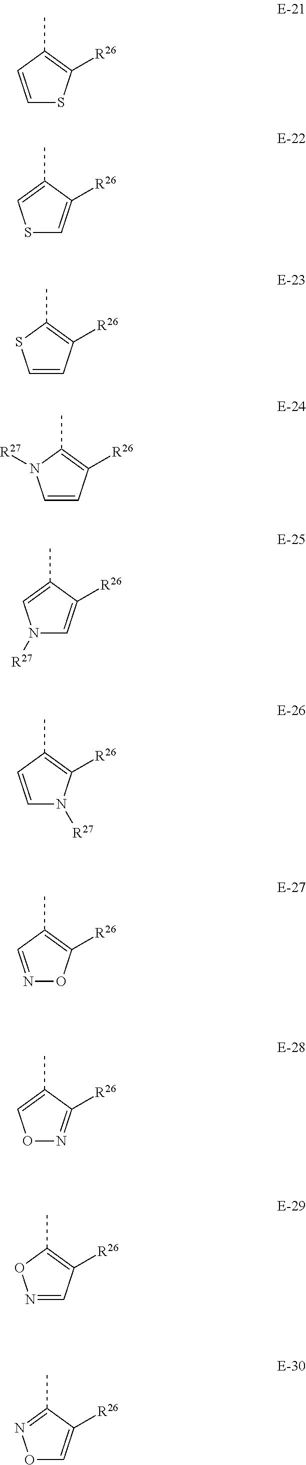

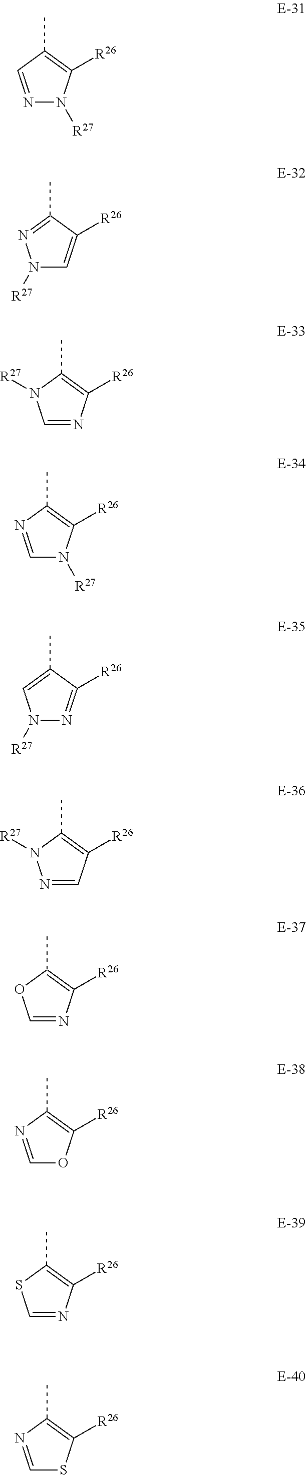

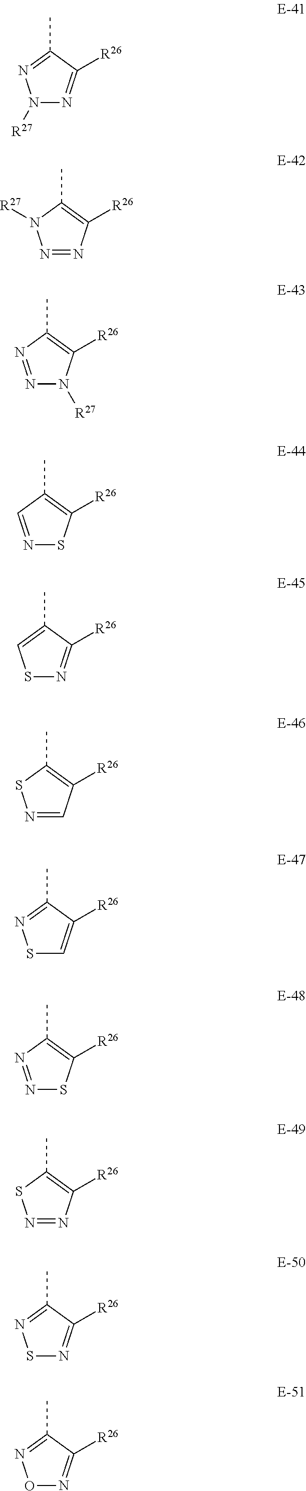

R²⁰ is a radical from the group of hydrogen, halogen, cyano, nitro, amino, hydroxyl, C₁-C₆-alkyl, C₁-C₆-haloalkyl, cyano-C₁-C₆-alkyl, C₂-C₆-alkenyl, C₂-C₆-alkynyl, C₃-C₆-cycloalkyl, C₃-C₆-cycloalkyl-C₁-C₆-alkyl, C₁-C₆-alkoxy, C₁-C₆-haloalkoxy, C₃-C₆-alkenyloxy, C₃-C₆-alkynyloxy, C₃-C₆-cycloalkyloxy, C₁-C₆-alkylcarbonyloxy, C₂-C₆-alkenylcarbonyloxy, C₂-C₆-alkynylcarbonyloxy, C₃-C₆-cycloalkylcarbonyloxy, C₁-C₆-alkoxycarbonyloxy, C₁-C₆-alkylsulphonyloxy, C₁-C₆-alkylamino, C₃-C₆-alkenylamino, C₃-C₆-alkynylamino, C₃-C₆-cycloalkylamino, C₁-C₆-alkylthio, C₁-C₆-haloalkylthio, C₃-C₆-alkenylthio, C₃-C₆-alkynylthio, C₃-C₆-cycloalkylthio, C₁-C₆-alkylsulphinyl, C₁-C₆-alkylsulphonyl, C₁-C₆-alkylcarbonyl, C₁-C₆-alkoxyimino-C₁-C₆-alkyl, C₁-C₆-alkoxycarbonyl, aminocarbonyl, C₁-C₆-alkylaminocarbonyl, di-(C₁-C₆)-alkylaminocarbonyl, aminothiocarbonyl, C₁-C₆-alkylaminosulphonyl, C₁-C₆-alkylsulphonylamino, C₁-C₆-alkylcarbonylamino, C₁-C₆-alkylthiocarbonylamino, of phenyl, phenoxy, pyridinyl and pyridinyloxy, each of which is optionally substituted by a radical from the group of halogen, cyano, nitro, amino, hydroxy, C₁-C₆-alkyl and C₁-C₆-haloalkyl,
R²¹ is a radical from the group of hydrogen, C₁-C₆-alkyl, C₃-C₆-cycloalkyl, C₁-C₆-haloalkyl, C₃-C₆-alkenyl, C₂-C₆-alkynyl, C₃-C₆-cycloalkyl-C₁-C₆-alkyl, cyano-C₁-C₆-alkyl, C₁-C₆-alkylcarbonyl, C₂-C₆-alkenylcarbonyl, C₁-C₆-haloalkylcarbonyl, C₂-C₆-haloalkenylcarbonyl, C₁-C₆-alkoxy-C₁-C₆-alkyl, C₁-C₆-alkoxycarbonyl, C₁-C₆-alkylsulphonyl and C₁-C₆-haloalkylsulphonyl,
R²³ is a radical from the group of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄-alkyl,
R²⁴ is hydrogen or is an in each case optionally halogen- or cyano-substituted radical from the group of C₁-C₄-alkyl, C₂-C₆-alkenyl, C₂-C₆-alkynyl, phenyl and phenyl-C₁-C₂-alkyl,
R²⁵ is hydrogen or is an in each case optionally halogen- or cyano-substituted radical from the group of C₁-C₄-alkyl, C₂-C₆-alkenyl, C₂-C₆-alkynyl, phenyl and phenyl-C₁-C₂-alkyl,
R²⁷ is hydrogen or C₁-C₄-alkyl and
R²⁶ is a radical from the group of hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylthio-C₁-C₂-alkyl, C₁-C₄-alkylsulphinyl-C₁-C₂-alkyl, C₁-C₄-alkylsulphonyl-C₁-C₂-alkyl and cyano-C₁-C₄-alkyl.

Area of preference (2): Particular preference is given to compounds of the formula (I) in which
A is a radical from the group of (A-a), (A-b) and (A-f)

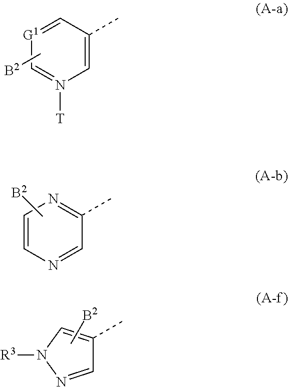

in which the broken line denotes the bond to the nitrogen atom of the bicyclic system of the formula (I),
G¹ is N or
B¹ is a radical from the group of hydrogen, halogen, C₁-C₆-alkyl and C₁-C₄-haloalkyl,
B² is a radical from the group of hydrogen, halogen, C₁-C₆-alkyl and C₁-C₄-haloalkyl,
T is oxygen or an electron pair,
R¹ is a radical from the group of hydrogen, C₁-C₄-alkyl and C₁-C₄-alkoxy,
R² a) is a B radical from the group of

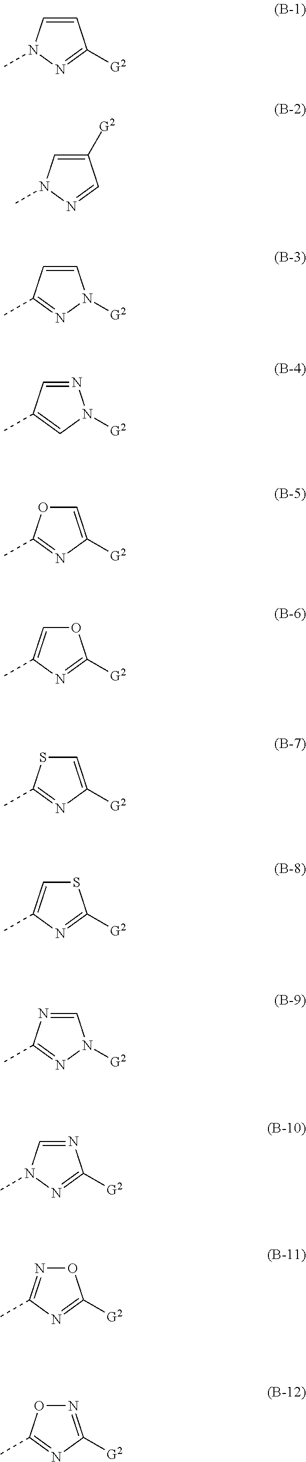

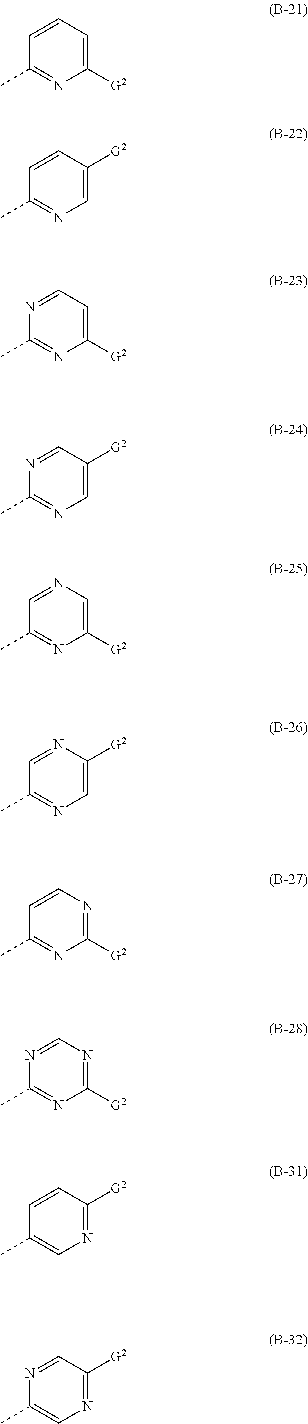

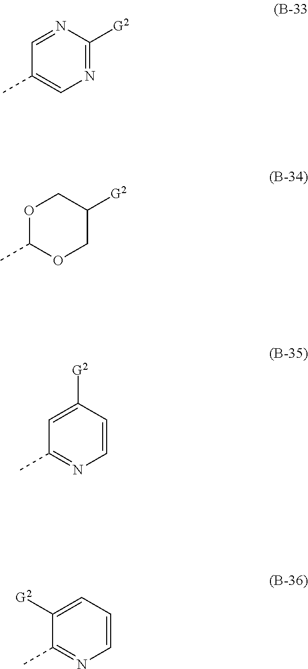

in which the broken line denotes the bond to the carbon atom of the bicyclic system of the formula (I), or
R² b) is a D radical from the group of (D-1) to (D-3)

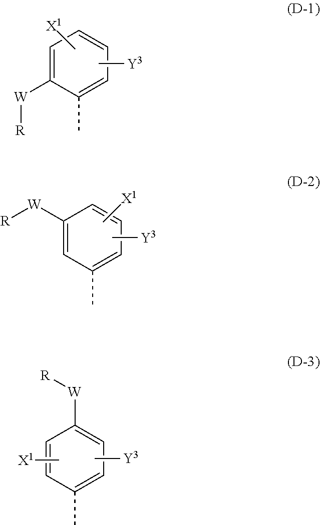

in which the broken line denotes the bond to the carbon atom of the bicyclic system of the formula (I), or
R² c) is a radical of the formula

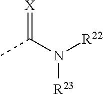

in which the broken line denotes the bond to the carbon atom of the bicyclic system of the formula (I), or
R² d) is a radical of the formula

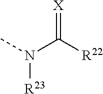

in which the broken line denotes the bond to the carbon atom of the bicyclic system of the formula (I), or
R² e) is an F radical from the group of (F-1), (F-8) and (F-10)

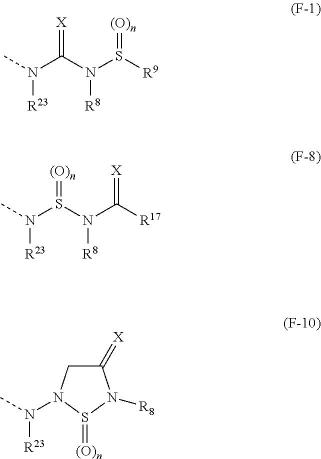

in which the broken line denotes the bond to the carbon atom in the formula (I) or
R² f) is a radical from the group of C₁-C₆-haloalkyl, carboxyl and amino,
   in which
G² is hydrogen or a radical from the group of halogen, nitro, amino, cyano, C₁-C₄-alkylamino, halo-C₁-C₄-alkylamino, C₁-C₄-dialkylamino, C₁-C₄-alkyl, C₁-C₄-alkoxycarbonyl-C₁-C₄-alkyl, halo-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy-(C₁-C₄-alkylsulphanyl)-C₁-C₄-alkyl, C₁-C₄-alkoxy-(C₁-C₄-alkylsulphinyl)-C₁-C₄-alkyl, C₁-C₄-alkoxy-(C₁-C₄-alkylsulphonyl)-C₁-C₄-alkyl, bis(C₁-C₄-alkylsulphanyl)-C₁-C₄-alkyl, bis(halo-C₁-C₄-alkylsulphanyl)-C₁-C₄-alkyl, bis(hydroxy-C₁-C₄-alkylsulphanyl)-C₁-C₄-alkyl, C₁-C₄-alkoxycarbonyl, C₁-C₄-alkoxycarbonyl-C₁-C₄-alkyl, C(X²)NR³R⁴, NR⁶1 e, C₁-C₄-alkylthio, C₁-C₄-alkylsulphinyl, C₁-C₄-alkylsulphonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may in turn be substituted by C₁-C₄-alkyl, halo-C₁-C₄-alkyl, C₁-C₄-alkoxy and C₁-C₄-alkoxy-C₁-C₄-alkyl), phenyl (which for its part may in turn be substituted by halogen, cyano, nitro, C₁-C₄-alkyl and halo-C₁-C₄-alkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may in turn be substituted by halogen, nitro, C₁-C₄-alkyl, halo-C₁-C₄-alkyl, C₁-C₄-alkoxy, halo-C₁-C₄-alkoxy, C₁-C₄-alkoxy-C₁-C₄-alkyl, C₁-C₄-alkylthio, C₁-C₄-alkylthio-C₁-C₄-alkyl and C₃-C₆-cyclo-C₁-C₄-alkyl) and the heteroaryl-C₁-C₄-alkyl radicals triazolyl-C₁-C₄-alkyl, pyridyl-C₁-C₄-alkyl, pyrimidyl-C₁-C₄-alkyl and oxadiazolyl-C₁-C₄-alkyl (which for their part may in turn be substituted by halogen and C₁-C₄-alkyl), or
G² is a C radical from the group of (C-1), (C-6) and (C-9)

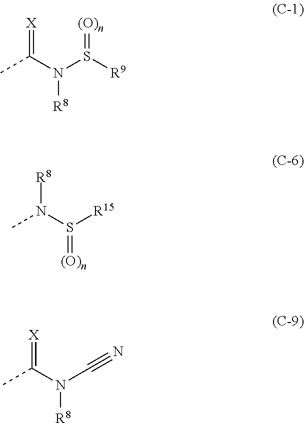

in which the broken line denotes the bond to the B radicals,
X is oxygen,
X¹ is a radical from the group of hydrogen, fluorine, chlorine, bromine, iodine, cyano, nitro, C₁-C₄-alkyl, C₁-C₄-haloalkyl, C₃-C₆-cycloalkyl, C₁-C₄-alkoxy and C₁-C₄-haloalkoxy,
X² is oxygen, sulphur, NR⁵ or NOH,
n is 2,
R is NR¹⁸R¹⁹ or is in each case optionally mono- to hepta-halogen-substituted, mono- or di-oxygen-substituted (leads to C═O) or mono- or di-cyano-substituted C₁-C₆-alkyl, C₃-C₆-alkenyl, C₃-C₆-alkynyl, C₁-C₄-alkoxy-C₁-C₃-alkyl, C₁-C₄-alkyl-S—C₁-C₃-alkyl, C₁-C₄-alkyl-S(O)—C₁-C₃-alkyl, C₁-C₄-alkyl-S(O)₂—C₁-C₃-alkyl, is R¹⁸—CO—C₁-C₂-alkyl, is NR¹⁸R¹⁹—CO—C₁-C₂-alkyl, is optionally mono- or di-oxygen- (leads to C═O), —C₁-C₄-alkyl-, —C₃-C₆-cycloalkyl-, —C₁-C₄-alkoxy- and —C₁-C₄-haloalkyl-substituted C₃-C₆-cycloalkyl, is optionally mono- or di-oxygen- (leads to C═O), —C₁-C₄-alkyl-, —C₃-C₆-cycloalkyl-, —C₁-C₄-alkoxy- and —C₁-C₄-haloalkyl-substituted C₃-C₈-cycloalkenyl, is optionally mono- or di-oxygen- (leads to C═O), —C₁-C₄-alkyl-, —C₃-C₆-cycloalkyl-, —C₁-C₄-alkoxy- and —C₁-C₄-haloalkyl-substituted C₃-C₆-cycloalkyl-C₁-C₄-alkyl, is optionally mono- or di-oxygen- (leads to C═O), —C₁-C₄-alkyl-, —C₃-C₆-cycloalkyl-, —C₁-C₄-alkoxy- and —C₁-C₄-haloalkyl-substituted C₃-C₆-cycloalkenyl-C₁-C₄-alkyl, is optionally mono- or di-oxygen- (leads to C═O), —C₁-C₄-alkyl-, —C₃-C₆-cycloalkyl-, —C₁-C₄-alkoxy- and —C₁-C₄-haloalkyl-substituted heterocyclyl, is optionally mono- or di-oxygen- (leads to C═O), —C₁-C₄-alkyl-, —C₃-C₆-cycloalkyl-, —C₁-C₄-alkoxy- and —C₁-C₄-haloalkyl-substituted heterocyclyl-C₁-C₄-alkyl or is in each case optionally mono- to tri-halogen-, -cyano-, —C₁-C₄-alkyl-, —C₁-C₄-haloalkyl-, —C₃-C₆-cycloalkyl-, —C₁-C₄-alkoxy- or —C₁-C₄-haloalkoxy-substituted phenyl, phenyl-C₁-C₄-alkyl, hetaryl and hetaryl-C₁-C₄-alkyl,
R³ is C₁-C₄-alkyl,
R⁴ is a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁵ is a radical from the group of hydrogen, C₁-C₄-alkyl, cyano-C₁-C₄-alkyl, C₂-C₄-alkynyl, C₃-C₆-cycloalkyl, C₁-C₄-alkoxycarbonyl, C₁-C₄-alkoxycarbonyl-C₁-C₄-alkyl and C₁-C₄-alkylthio-C₁-C₄-alkyl,
R⁶ is hydrogen or C₁-C₄-alkyl,
R⁷ is a radical from the group of hydrogen, C₁-C₄-alkyl, cyano-C₁-C₄-alkyl, C₂-C₄-alkynyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, aryl-C₁-C₄-alkyl and hetaryl-C₁-C₄-alkyl or
R⁶ and R⁷ together with the nitrogen atom to which they are bonded form a 4- to 7-membered ring which may contain one or two further heteroatoms from the group of nitrogen, oxygen and sulphur (where oxygen and sulphur atoms must not be directly adjacent to one another),
R⁸ is a radical from the group of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulphonyl, optionally halogen-substituted C₁-C₆-alkoxycarbonyl and optionally halogen-, C₁-C₆-alkyl-, C₁-C₆-alkoxy-, C₁-C₆-haloalkyl- and cyano-substituted C₃-C₆-cycloalkylcarbonyl, or is a cation or an optionally C₁-C₆-alkyl- or aryl-C₁-C₆-alkyl-substituted ammonium ion,

R⁹ is a radical from the group of in each case optionally halogen-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl- and C₁-C₄-haloalkylsulphonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, in which one or two ring members may each be replaced by a heteroatom from the group of sulphur, oxygen (where oxygen atoms must not be directly adjacent to one another) and nitrogen (and in this case is especially

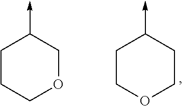

where the arrow in each case marks the bond to the sulphur atom in the (C-1) radical and in the (F-1) radical), in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl-, C₁-C₄-haloalkylsulphonyl-, amino-, C₁-C₄-alkylamino-, C₁-C₄-alkylcarbonylamino-, C₁-C₄-alkoxycarbonylamino-, C₁-C₄-alkoxy-C₁-C₄-alkyl, C₁-C₄-haloalkoxy-C₁-C₄-alkyl, C₂-C₄-alkenyl-, C₂-C₄-alkynyl-, C₁-C₄-alkylcarbonyl-, C₁-C₄-alkoxycarbonyl- or aminocarbonyl-substituted aryl, heteroaryl, alkyl and heteroaryl-C₁-C₄-alkyl, or is NR′R″ in which R′ and R″ are independently a radical from the group of hydrogen and C₁-C₄-alkyl, or
R⁸ and R⁹ in the (C-1) radical and in the (F-1) radical, together with the N—S(O)n group to which they are bonded, may also form a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group of sulphur, oxygen (where oxygen atoms must not be directly adjacent to one another) and nitrogen and/or at least one and preferably exactly one carbonyl group; in particular R⁸ and R⁹ together with the N—S(O)n group to which they are bonded may be a radical from the group of

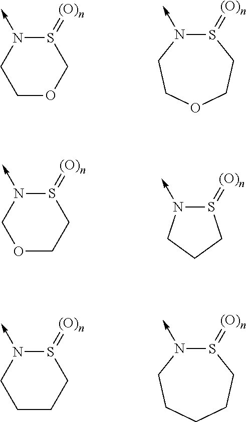

(in which the arrow in each case marks the bond to the C(X) group),
R¹⁵ is a radical from the group of in each case optionally methyl-substituted C₁-C₆-alkyl, C₂-C₆-alkenyl and C₂-C₆-alkynyl, in each case optionally methyl-, halogen-, cyano- or carbamoyl-substituted C₃-C₆-cycloalkyl, C₃-C₆-cycloalkyl-C₁-C₂-alkyl and C₃-C₆-cycloalkenyl,
R⁸ and R¹⁵ in the (C-6) radical, together with the N—S(O)n group to which they are bonded, may also form a saturated or unsaturated and optionally substituted 4- to 8-membered ring which may contain one or two further heteroatoms from the group of sulphur, oxygen (where oxygen and sulphur atoms must not be directly adjacent to one another) and nitrogen and/or at least one carbonyl group,
R¹⁷ is a radical from the group of in each case optionally halogen-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl- and C₁-C₄-haloalkylsulphonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl-, C₁-C₄-haloalkylsulphonyl-, amino-, C₁-C₄-alkylamino-, di-(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl and heteroaryl-C₁-C₄-alkyl, or is NR′R″ in which R′ and R″ are independently a radical from the group of hydrogen and C₁-C₄-alkyl,
R¹⁸ is a radical from the group of hydrogen, hydroxyl, in each case optionally mono- or poly-halogen-substituted or mono- or di-cyano-substituted C₁-C₄-alkyl, C₁-C₄-alkoxy, C₁-C₄-alkoxy-C₁-C₄-alkyl, C₁-C₄-alkyl-S—C₁-C₃-alkyl, C₁-C₄-alkyl-S(O)—C₁-C₃-alkyl, C₁-C₄-alkyl-S(O)₂—C₁-C₃-alkyl, C₁-C₄-alkylcarbonyl, C₁-C₄-alkoxy carbonyl, C₃-C₆-cycloalkyl, C₃-C₆-cycloalkyl-C₁-C₃-alkyl, heterocyclyl, heterocyclyl-C₁-C₃-alkyl and in each case optionally mono- to tri-C₁-C₄-alkyl-, —C₁-C₃-haloalkyl-, —C₁-C₃-alkoxy-, —C₁-C₃-haloalkoxy-, -cyclopropyl-, -fluorine-, -chlorine-, -bromine- or -cyano-substituted phenyl, benzyl, pyridyl, pyrimidyl, thiazolyl, oxazolyl, pyrazolyl, thienyl, furanyl, pyridinylmethyl and thiazolylmethyl,
R¹⁹ is hydrogen, is an alkali metal or alkaline earth metal ion, or is an optionally mono- to tetra-C₁-C₄-alkyl-substituted ammonium ion or is an in each case optionally mono- or poly-halogen-substituted or mono- or di-cyano-substituted radical from the group of C₁-C₄-alkyl, C₁-C₄-alkoxy, C₁-C₄-alkoxy-C₁-C₂-alkyl, C₁-C₄-alkyl-S—C₁-C₂-alkyl, C₁-C₄-alkyl-S(O)—C₁-C₂-alkyl and C₁-C₄-alkyl-S(O)₂—C₁-C₂-alkyl,
Y³ is a radical from the group of hydrogen, fluorine, chlorine, bromine, iodine, cyano, C₁-C₄-alkyl, C₁-C₄-haloalkyl, C₃-C₆-cycloalkyl, C₁-C₄-alkoxy and C₁-C₄-haloalkoxy,
W is a radical from the group of S, SO and SO₂,
R²², when R² is the radical c), is a radical from the group of C₁-C₆-alkyl, optionally halogen-, carbamoyl-, thiocarbamoyl- or cyano-substituted C₃-C₆-cycloalkyl, C₁-C₆-haloalkyl, C₁-C₆-alkoxy, C₁-C₄-haloalkoxy, C₁-C₆-alkoxy-C₁-C₆-alkyloxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylthio-C₁-C₄-alkyl, C₁-C₄-alkylsulphinyl-C₁-C₄-alkyl, C₁-C₄-alkylsulphonyl-C₁-C₄-alkyl, C₁-C₄-alkylthio-C₁-C₄-alkyloxy, C₁-C₄-alkylsulphinyl-C₁-C₄-alkyloxy, C₁-C₄-alkylsulphonyl-C₁-C₄-alkyloxy, C₁-C₄-haloalkylthio-C₁-C₄-alkyl, C₁-C₄-haloalkylsulphinyl-C₁-C₄-alkyl, C₁-C₄-haloalkylsulphonyl-C₁-C₄-alkyl, C₁-C₄-alkylthio-C₂-C₄-alkenyl, C₁-C₄-alkylsulphinyl-C₂-C₄-alkenyl, C₁-C₄-alkylsulphonyl-C₂-C₄-alkenyl, C₂-C₄-alkenylthio-C₁-C₄-alkyl, C₂-C₄-alkenylsulphinyl-C₁-C₄-alkyl, C₂-C₄-alkenylsulphonyl-C₁-C₄-alkyl, C₁-C₄-alkylcarbonyl-C₁-C₄-alkyl, C₁-C₄-haloalkylcarbonyl-C₁-C₄-alkyl, C₁-C₄-alkoxy-C₁-C₄-alkyl, C₁-C₄-haloalkoxy-C₁-C₄-alkyl, C₁-C₄-alkoxy carbonyl-C₁-C₄-alkyl, C₁-C₄-haloalkoxy carbonyl-C₁-C₄-alkyl, C₁-C₄-alkylaminosulphonyl, di-(C₁-C₄-alkyl)-aminosulphonyl,

R²³, when R² is the radical c), is a radical from the group of hydrogen, C₁-C₆-alkyl, C₂-C₄-alkenyl, C₂-C₄-alkynyl, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄-alkyl,
R²³, when R² is the radical d) or e), is a radical from the group of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄-alkyl,
   or in the case that R²=d)
R²² is also optionally halogen-, cyano-, nitro-, amino-, hydroxyl-, C₁-C₆-alkyl-, C₁-C₆-haloalkyl-, C₃-C₆-cycloalkyl-, C₁-C₆-alkoxy-, C₁-C₄-haloalkoxy-, C₁-C₆-alkoxycarbonyloxy-, C₁-C₆-alkylamino-, C₁-C₆-alkylthio-, C₁-C₄-haloalkylthio-, C₁-C₄-alkylsulphinyl-, C₁-C₄-alkylsulphonyl-, C₁-C₆-alkylcarbonyl-, aminocarbonyl-, C₁-C₆-alkylaminocarbonyl-, di-(C₁-C₆-alkyl)-aminocarbonyl-, C₁-C₆-alkylcarbonylamino-substituted phenyl or is an E radical from the group of

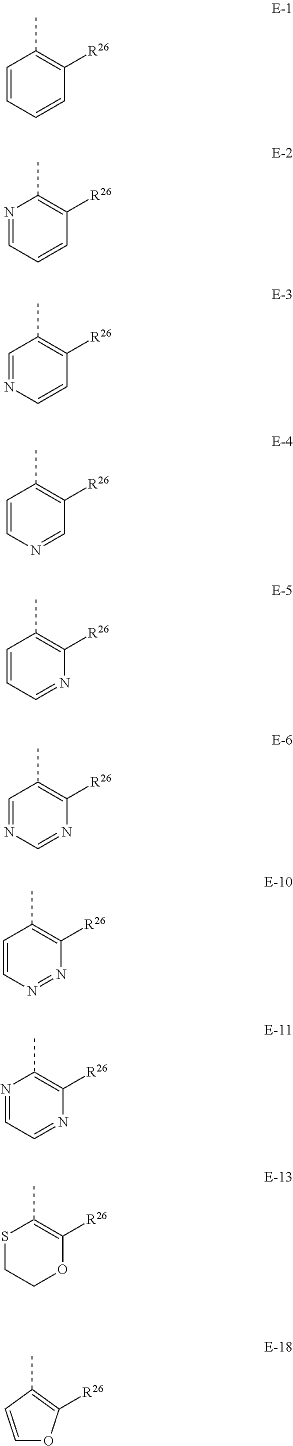

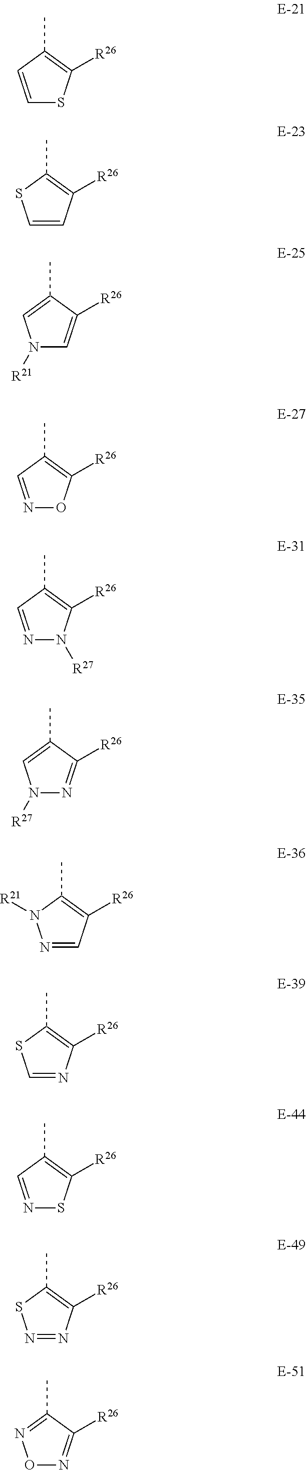

R²⁷ is hydrogen or C₁-C₄-alkyl and
R²⁶ is a radical from the group of hydrogen, C₁-C₄-alkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylthio-C₁-C₄-alkyl, C₁-C₄-alkylsulphonyl-C₁-C₂-alkyl and cyano-C₁-C₄-alkyl.

Area of preference (3): Very particular preference is given to compounds of the formula (I) in which
A is a radical from the group of (A-a), (A-b) and (A-f)

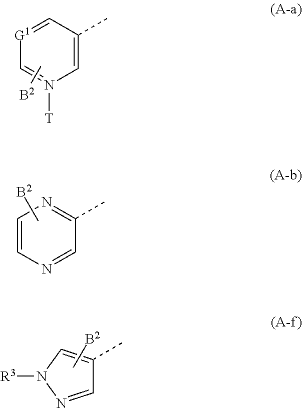

in which the broken line denotes the bond to the nitrogen atom of the bicyclic system of the formula (I),
G¹ is N or C—B¹,
B¹ is a radical from the group of hydrogen and fluorine,
B² is hydrogen,
T is oxygen or an electron pair,
R¹ is hydrogen,
R² is a B radical from the group of

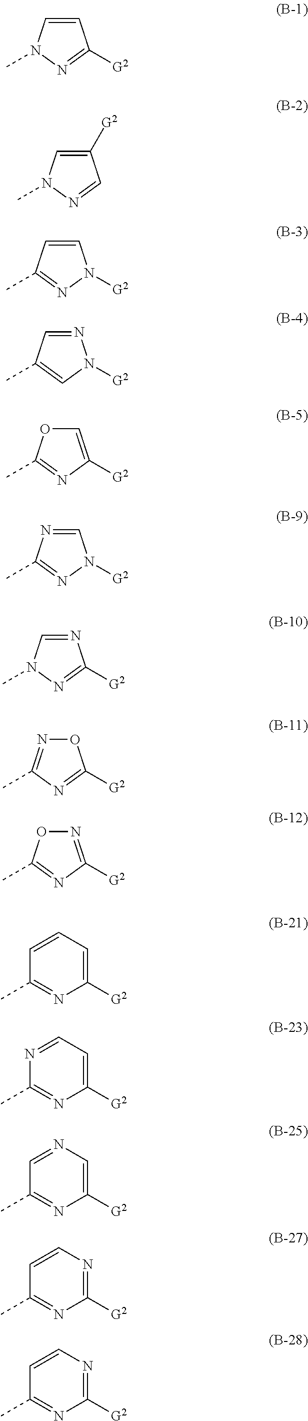

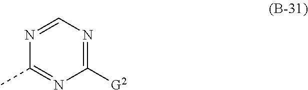

in which the broken line denotes the bond to the carbon atom of the bicyclic system of the formula (I), or
R² b) is a D radical from the group of (D-1) to (D-3)

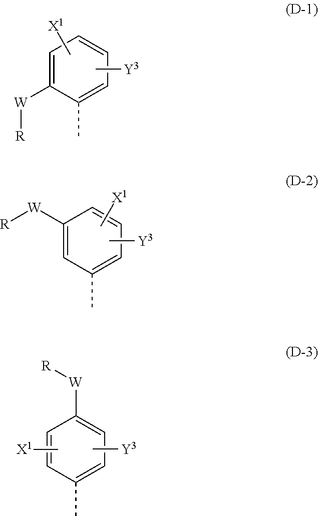

in which the broken line denotes the bond to the carbon atom of the bicyclic system of the formula (I),
or
R² c) is a radical of the formula

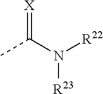

in which the broken line denotes the bond to the carbon atom of the bicyclic system of the formula (I), or
R² d) is a radical of the formula

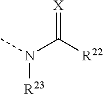

in which the broken line denotes the bond to the carbon atom of the bicyclic system of the formula (I), or
R² e) is an F radical from the group of (F-1), (F-8) and (F-10)

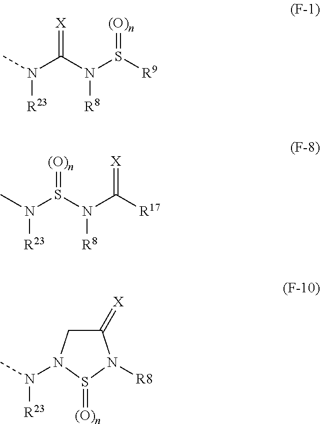

in which the broken line denotes the bond to the carbon atom of the bicyclic system of the formula (I), or
R² f) is a radical from the group of C₁-C₆-haloalkyl, carboxyl and amino, in which
G² is hydrogen or a radical from the group of halogen, nitro, amino, cyano, C₁-C₄-alkylamino, halo-C₁-C₄-alkylamino, C₁-C₄-dialkylamino, C₁-C₄-alkyl, C₁-C₄-alkoxycarbonyl-C₁-C₄-alkyl, halo-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy(C₁-C₄-alkylsulphanyl)-C₁-C₄-alkyl, C₁-C₄-alkoxy(C₁-C₄-alkylsulphinyl)-C₁-C₄-alkyl, C₁-C₄-alkoxy(C₁-C₄-alkylsulphonyl)-C₁-C₄-alkyl, bis(C₁-C₄-alkylsulphanyl)-C₁-C₄-alkyl, bis(halo-C₁-C₄-alkylsulphanyl)-C₁-C₄-alkyl, bis(hydroxy-C₁-C₄-alkylsulphanyl)-C₁-C₄-alkyl, C₁-C₄-alkoxycarbonyl, C₁-C₄-alkoxycarbonyl-C₁-C₄-alkyl, C(X²)NR³R⁴, NR⁶R⁷, C₁-C₄-alkylthio, C₁-C₄-alkylsulphinyl and C₁-C₄-alkylsulphonyl, or
G² is a C radical (C-1) or (C-9)

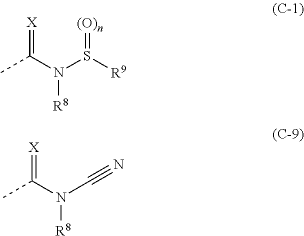

in which the broken line denotes the bond to the B radicals,
X is oxygen,
X¹ is a radical from the group of hydrogen, fluorine, chlorine, bromine, cyano, methyl, ethyl, trifluoromethyl, methoxy, ethoxy, difluoromethoxy and trifluoromethoxy,
X² is oxygen, sulphur, NR⁵ or NOH,
n is 2,
R is NR¹⁸R¹⁹ or is an in each case optionally mono-, di-, tri-, tetra- or penta-fluorine- or -chlorine-substituted or mono- or di-cyano-substituted radical from the group of C₁-C₄-alkyl, C₃-C₄-alkenyl, C₃-C₄-alkynyl, C₁-C₂-alkoxy-C₁-C₂-alkyl and C₁-C₂-alkyl-S—C₁-C₂-alkyl, C₁-C₂-alkyl-S(O)—C₁-C₂-alkyl, C₁-C₂-alkyl-S(O)₂—C₁-C₂-alkyl, is R¹⁸—CO—C₁-C₂-alkyl, is NR¹⁸R¹⁹—CO—C₁-C₂-alkyl, is C₃-C₆-cycloalkyl optionally mono- or disubstituted by C₁-C₂-alkyl, C₁-C₂-alkoxy or C₁-C₂-haloalkyl or by an oxygen atom (leads to C═O), is C₃-C₆-cycloalkenyl optionally mono- or disubstituted by C₁-C₂-alkyl, C₁-C₂-alkoxy or C₁-C₂-haloalkyl or by an oxygen atom (leads to C═O), is C₃-C₆-cycloalkyl-C₁-C₂-alkyl optionally mono- or disubstituted by C₁-C₂-alkyl, C₁-C₂-alkoxy or C₁-C₂-haloalkyl, is C₃-C₆-cycloalkenyl-C₁-C₂-alkyl optionally mono- or disubstituted by C₁-C₂-alkyl, C₁-C₂-alkoxy or C₁-C₂-haloalkyl, is heterocyclyl optionally mono- or disubstituted by C₁-C₂-alkyl, C₁-C₂-alkoxy or C₁-C₂-haloalkyl, is heterocyclyl-C₁-C₂-alkyl optionally mono- or disubstituted by C₁-C₂-alkyl, C₁-C₂-alkoxy or C₁-C₂-haloalkyl, or is in each case optionally mono- or di-fluorine-,
   -chlorine-, -bromine-, -cyano-, -methyl-, -ethyl-, -difluoromethyl-, -trifluoromethyl-, -methoxy-, -ethoxy-, -difluoromethoxy- or -trifluoromethoxy-substituted phenyl, benzyl, pyridyl, pyrimidyl, thiazolyl, oxazolyl, pyrazolyl, thienyl, furanyl, pyridinylmethyl or thiazolylmethyl,
R³ is C₁-C₄-alkyl,
R⁴ is a radical from the group of hydrogen, C₁-C₄-alkyl, cyano-C₁-C₄-alkyl, C₂-C₄-alkynyl, C₃-C₆-cycloalkyl, C₁-C₄-alkoxycarbonyl, C₁-C₄-alkoxycarbonyl-C₁-C₄-alkyl and C₁-C₄-alkylthio-C₁-C₄-alkyl,
R⁵ is a radical from the group of hydrogen, C₁-C₄-alkyl, cyano-C₁-C₄-alkyl, C₂-C₄-alkynyl, C₃-C₆-cycloalkyl, C₁-C₄-alkoxycarbonyl, C₁-C₄-alkoxycarbonyl-C₁-C₄-alkyl and C₁-C₄-alkylthio-C₁-C₄-alkyl,
R⁶ is hydrogen or C₁-C₄-alkyl,
R⁷ is a radical from the group of hydrogen, C₁-C₄-alkyl, cyano-C₁-C₄-alkyl, C₂-C₄-alkynyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, aryl-C₁-C₄-alkyl and hetaryl-C₁-C₄-alkyl or
R⁶ and R⁷ together with the nitrogen atom to which they are bonded form a 4- to 7-membered ring which may contain one or two further heteroatoms from the group of nitrogen, oxygen and sulphur (where oxygen and sulphur atoms must not be directly adjacent to one another),
R⁸ is a radical from the group of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulphonyl, optionally halogen-substituted C₁-C₆-alkoxycarbonyl, optionally halogen-, C₁-C₆-alkyl-, C₁-C₆-alkoxy-, C₁-C₆-haloalkyl- and cyano-substituted C₃-C₆-cycloalkylcarbonyl, or is a cation or an optionally C₁-C₆-alkyl- or aryl-C₁-C₆-alkyl-substituted ammonium ion,
R⁹ is a radical from the group of in each case optionally halogen-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl- and C₁-C₄-haloalkylsulphonyl-substituted C₁-C₄-alkyl, C₂-C₄-alkenyl and C₂-C₄-alkynyl, in each case optionally halogen-, C₁-C₄-alkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, in which one or two ring members may each be replaced by a heteroatom from the group of sulphur, oxygen (where oxygen atoms must not be directly adjacent to one another) and nitrogen (and in this case is especially

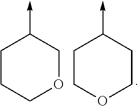

where the arrow in each case marks the bond to the sulphur atom in the (C-1) radical and in the (F-1) radical), in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl-, C₁-C₄-haloalkylsulphonyl-, amino-, C₁-C₄-alkylamino-, di(C₁-C₄-alkyl)amino-, C₁-C₄-alkylcarbonylamino-, C₁-C₄-alkoxy carbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl and heteroaryl-C₁-C₄-alkyl, or is NR′R″ in which R′ and R″ are independently a radical from the group of hydrogen and C₁-C₄-alkyl,
R⁸ and R⁹ in the (C-1) radical and in the (F-1) radical, together with the N—S(O)n group to which they are bonded, may also form a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group of sulphur, oxygen (where oxygen atoms must not be directly adjacent to one another) and nitrogen and/or at least one and preferably one carbonyl group; in particular, R⁸ and R⁹ together with the N—S(O)n group to which they are bonded may be a radical from the group of

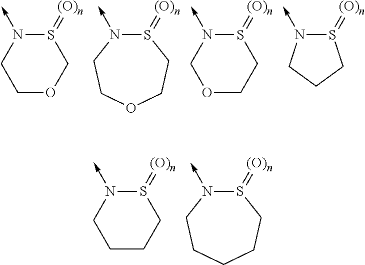

(in which the arrow in each case marks the bond to the C(X) group),
R¹⁷ is a radical from the group of in each case optionally halogen-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl- and C₁-C₄-haloalkylsulphonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl-, C₁-C₄-haloalkylsulphonyl-, amino-, C₁-C₄-alkylamino-, di-(C₁-C₄-alkyl)-amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl and heteroaryl-C₁-C₄-alkyl, or is NR′R″ in which R′ and R″ are independently a radical from the group of hydrogen and C₁-C₄-alkyl,
W is a radical from the group of S, SO and SO₂,
Y³ is a radical from the group of hydrogen, fluorine, chlorine, bromine, cyano, methyl, ethyl, trifluoromethyl, methoxy, ethoxy, difluoromethoxy and trifluoromethoxy,
R¹⁸ is a radical from the group of hydrogen, hydroxyl, in each case optionally mono-, di-, tri-, tetra- or penta-fluorine- or -chlorine-substituted or mono- or di-cyano-substituted radical from the group of C₁-C₄-alkyl, C₁-C₄-alkoxy, C₁-C₄-alkoxy-C₁-C₄-alkyl, C₁-C₄-alkyl-S—C₁-C₂-alkyl, C₁-C₄-alkyl-S(O)—C₁-C₂-alkyl, C₁-C₄-alkyl-S(O)₂—C₁-C₂-alkyl, C₁-C₄-alkylcarbonyl, C₁-C₄-alkoxycarbonyl, C₃-C₆-cycloalkyl, C₃-C₆-cycloalkyl-C₁-C₃-alkyl, heterocyclyl and heterocyclyl-C₁-C₃-alkyl, and in each case optionally mono- to tri-C₁-C₄-alkyl-, —C₁-C₃-haloalkyl-, —C₁-C₃-alkoxy-, —C₁-C₃-haloalkoxy-, -cyclopropyl-, -fluorine-, -chlorine-, -bromine- or -cyano-substituted phenyl, benzyl, pyridyl, pyrimidyl, thiazolyl, oxazolyl, pyrazolyl, thienyl, furanyl, pyridinylmethyl and thiazolylmethyl,
R¹⁹ is hydrogen, is an alkali metal or alkaline earth metal ion, is an optionally mono- to tetra-C₁-C₄-alkyl-substituted ammonium ion or is an in each case optionally mono-, di-, tri-, tetra- or penta-fluorine- or -chlorine-substituted or mono- or di-cyano-substituted radical from the group of C₁-C₄-alkyl, C₁-C₄-alkoxy, C₁-C₄-alkoxy-C₁-C₂-alkyl, C₁-C₄-alkyl-S—C₁-C₂-alkyl, C₁-C₄-alkyl-S(O)—C₁-C₂-alkyl and C₁-C₄-alkyl-S(O)₂—C₁-C₂-alkyl,
R²², when R² is the radical c), is a radical from the group of C₁-C₆-alkyl, optionally cyano-substituted C₃-C₆-cycloalkyl, C₁-C₆-haloalkyl, C₁-C₆-alkoxy, C₁-C₄-haloalkoxy, C₁-C₆-alkoxy-C₁-C₆-alkyloxy, C₁-C₄-alkylthio, C₁-C₄-alkylsulphinyl, C₁-C₄-alkylsulphonyl, C₁-C₄-haloalkylthio, C₁-C₄-haloalkylsulphinyl, C₁-C₄-haloalkylsulphonyl, C₁-C₄-alkylthio-C₁-C₄-alkyl, C₁-C₄-alkylsulphinyl-C₁-C₄-alkyl, C₁-C₄-alkylsulphonyl-C₁-C₄-alkyl, C₁-C₄-alkylthio-C₁-C₄-alkyloxy, C₁-C₄-alkylsulphinyl-C₁-C₄-alkyloxy, C₁-C₄-alkylsulphonyl-C₁-C₄-alkyloxy, C₁-C₄-haloalkylthio-C₁-C₄-alkyl, C₁-C₄-haloalkylsulphinyl-C₁-C₄-alkyl, C₁-C₄-haloalkylsulphonyl-C₁-C₄-alkyl, C₁-C₄-alkylthio-C₂-C₄-alkenyl, C₁-C₄-alkylsulphinyl-C₂-C₄-alkenyl, C₁-C₄-alkylsulphonyl-C₂-C₄-alkenyl, C₂-C₄-alkenylthio-C₁-C₄-alkyl, C₂-C₄-alkenylsulphinyl-C₁-C₄-alkyl, C₂-C₄-alkenylsulphonyl-C₁-C₄-alkyl, C₁-C₄-alkylcarbonyl-C₁-C₄-alkyl, C₁-C₄-haloalkylcarbonyl-C₁-C₄-alkyl, C₁-C₄-alkoxy-C₁-C₄-alkyl, C₁-C₄-haloalkoxy-C₁-C₄-alkyl, C₁-C₄-alkoxycarbonyl-C₁-C₄-alkyl, C₁-C₄-haloalkoxycarbonyl-C₁-C₄-alkyl, C₁-C₄-alkylaminosulphonyl, di-(C₁-C₄-alkyl)-aminosulphonyl,
R²³, when R² is the radical c), is a radical from the group of hydrogen, C₁-C₆-alkyl, C₂-C₄-alkenyl, C₂-C₄-alkynyl, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄-alkyl,
R²², when R² is the radical d), is a radical from the group of C₁-C₄-alkyl, optionally cyano-substituted C₃-C₆-cycloalkyl, C₁-C₄-haloalkyl, C₁-C₄-alkylthio-C₁-C₄-alkyl, C₁-C₄-alkylsulphinyl-C₁-C₄-alkyl, C₁-C₄-alkylsulphonyl-C₁-C₄-alkyl, C₁-C₄-haloalkylthio-C₁-C₄-alkyl, C₁-C₄-haloalkylsulphinyl-C₁-C₄-alkyl, C₁-C₄-haloalkylsulphonyl-C₁-C₄-alkyl, C₁-C₄-alkylthio-C₂-C₄-alkenyl, C₁-C₄-alkylsulphinyl-C₂-C₄-alkenyl, C₁-C₄-alkylsulphonyl-C₂-C₄-alkenyl, C₂-C₄-alkenylthio-C₁-C₄-alkyl, C₂-C₄-alkenylsulphinyl-C₁-C₄-alkyl, C₂-C₄-alkenylsulphonyl-C₁-C₄-alkyl, C₁-C₄-alkylcarbonyl-C₁-C₄-alkyl, C₁-C₄-haloalkylcarbonyl-C₁-C₄-alkyl, C₁-C₄-alkoxy-C₁-C₄-alkyl, C₁-C₄-haloalkoxy-C₁-C₄-alkyl, C₁-C₄-alkoxycarbonyl-C₁-C₄-alkyl, C₁-C₄-haloalkoxycarbonyl-C₁-C₄-alkyl, C₁-C₄-alkylaminosulphonyl, di-(C₁-C₄-alkyl)-aminosulphonyl,
R²³, when R² is the radical d) or e), is a radical from the group of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄-alkyl, and in the case that R²=d)
R²² is also optionally halogen-, cyano-, nitro-, amino-, hydroxyl-, C₁-C₆-alkyl-, C₁-C₆-haloalkyl-, C₂-C₆-alkenyl-, C₂-C₆-alkynyl-, C₃-C₆-cycloalkyl-, C₁-C₆-alkoxy-, C₁-C₆-haloalkoxy-, C₃-C₆-alkenyloxy-, C₃-C₆-alkynyloxy-, C₁-C₆-alkoxycarbonyloxy-, C₁-C₆-alkylamino-, C₃-C₆-alkenylamino-, C₃-C₆-alkynylamino-, C₃-C₆-cycloalkylamino-, C₁-C₆-alkylthio-, C₁-C₆-haloalkylthio-, C₃-C₆-alkenylthio-, C₃-C₆-alkynylthio-, C₃-C₆-cycloalkylthio-, C₁-C₆-alkylsulphinyl-, C₁-C₆-alkylsulphonyl-, C₁-C₆-alkylcarbonyl-, aminocarbonyl-, C₁-C₆-alkylaminocarbonyl-, di-(C₁-C₆-alkyl)-aminocarbonyl-, C₁-C₆-alkylcarbonylamino-substituted phenyl or is an E radical from the group of

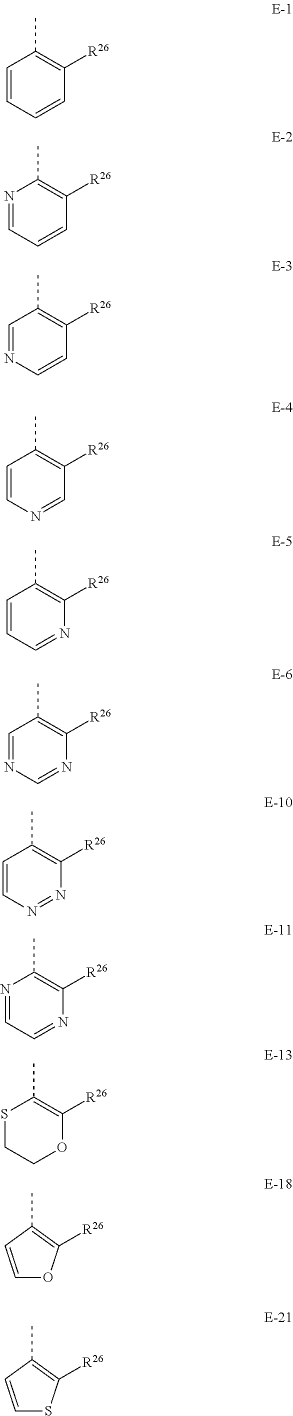

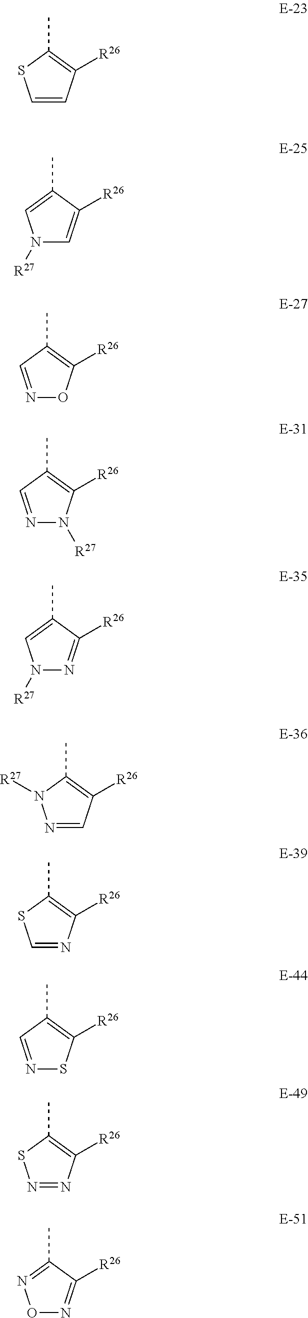

R²⁷ is hydrogen or methyl and
R²⁶ is a radical from the group of hydrogen, methyl, 2,2,2-trifluoroethyl, 2,2-difluoroethyl, propenyl, propargyl, cyclopropyl, cyclopropylmethyl, methoxymethyl, methylthioethyl, methylsulphinylethyl, methylsulphonylethyl and cyanomethyl.

Area of preference (4): A particular group of compounds of the formula (I) is that of those in which
A is the radical

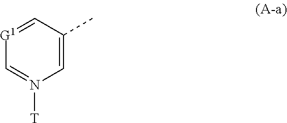

in which the broken line denotes the bond to the nitrogen atom of the bicyclic system of the formula (I),
G¹ is C—B¹,
B¹ is hydrogen,
T is an electron pair,
R¹ is hydrogen,
R² a) is

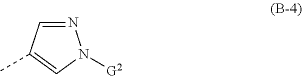

in which the broken line denotes the bond to the carbon atom of the bicyclic system of the formula (I), or
R² b) is the (D-2) radical

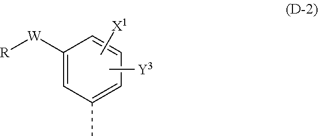

in which the broken line denotes the bond to the carbon atom of the bicyclic system of the formula (I), or
R² c) is the radical of the formula

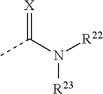

in which the broken line denotes the bond to the carbon atom of the bicyclic system of the formula (I), or
G² is a radical from the group of hydrogen and C₁-C₄-alkyl,
X is oxygen,
X¹ is a radical from the group of hydrogen, fluorine, chlorine and bromine,
R is optionally mono-, di-, tri-, tetra- or penta-fluorine- or -chlorine-substituted C₁-C₄-alkyl,
W is a radical from the group of S, SO and SO₂,
Y³ is methyl or ethyl,
R²² is a radical from the group of C₁-C₆-alkyl, C₁-C₆-haloalkyl and C₁-C₄-alkylthio-C₁-C₄-alkyl and R²³ is hydrogen or C₁-C₆-alkyl.

When sulphur and/or nitrogen occur in rings in the above definitions, for example in expressions such as “in which the rings may contain at least one heteroatom from the group of sulphur, oxygen (where oxygen and sulphur atoms must not be directly adjacent) and nitrogen” or “in which one or two ring members may each be replaced by a heteroatom from the group of sulphur, oxygen (where oxygen atoms must not be directly adjacent) and nitrogen”, unless stated otherwise, the sulphur may also be present in the form of SO or SO₂, and the nitrogen, if it is not in the form of —N═, as well as NH, may also be present in the form of N-alkyl (especially N—C₁-C₆-alkyl).

In the preferred definitions, the combination of which forms the area of preference (1), unless stated otherwise,

cation is an alkali metal ion selected from the group of lithium, sodium, potassium, rubidium, caesium, preferably from the group of lithium, sodium, potassium, or an
alkaline earth metal ion selected from the group of beryllium, magnesium, calcium, strontium, barium, preferably from the group of magnesium and calcium,
halogen is selected from the group of fluorine, chlorine, bromine and iodine, preferably in turn from the group of fluorine, chlorine and bromine,
aryl (including as part of a larger unit, for example arylalkyl) is selected from the group of phenyl, naphthyl, anthryl, phenanthrenyl, and preferably in turn is phenyl,
hetaryl (synonymous with heteroaryl, including as part of a larger unit, for example hetarylalkyl) is selected from the group of furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzoisofuryl, benzothienyl, benzoisothienyl, indolyl, isoindolyl, indazolyl, benzothiazolyl, benzoisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl,
heterocyclyl is a saturated 4-, 5- or 6-membered ring containing 1 or 2 nitrogen atoms and/or one oxygen atom and/or one sulphur atom, for example azetidinyl, azolidinyl, azinanyl, oxetanyl, oxolanyl, oxanyl, dioxanyl, thiethanyl, thiolanyl, thianyl, tetrahydrofuryl, piperazinyl, morpholinyl.

In the particularly preferred definitions whose combination forms the range of preference (2), unless stated otherwise,

cation is an alkali metal ion selected from the group of lithium, sodium, potassium, rubidium, caesium, preferably from the group of lithium, sodium, potassium, or an
alkaline earth metal ion selected from the group of beryllium, magnesium, calcium, strontium, barium, preferably from the group of magnesium and calcium,
halogen is selected from the group of fluorine, chlorine, bromine and iodine, preferably in turn from the group of fluorine, chlorine and bromine,
aryl (including as part of a larger unit, for example arylalkyl) is selected from the group of phenyl, naphthyl, anthryl, phenanthrenyl, and preferably in turn is phenyl,
hetaryl (synonymous with heteroaryl, also as part of a larger unit, for example hetarylalkyl) is selected from the group of pyrazolyl, imidazolyl, 1,2,3-triazolyl, 1,2,4-triazolyl, oxazolyl, isoxazolyl, thiazolyl, isothiazolyl, pyridyl, pyrimidinyl, pyridazinyl, pyrazinyl, 1,2,3-triazinyl, 1,2,4-triazinyl, 1,3,5-triazinyl, heterocyclyl is selected from the group of azetidinyl, azolidinyl, azinanyl, oxetanyl, oxolanyl, oxanyl, dioxanyl, thiethanyl, thiolanyl, thianyl, tetrahydrofuryl, piperazinyl, morpholinyl.

In the very particularly preferred definitions and the especially preferred definitions whose combination forms the range of preference (3), unless stated otherwise,

cation is an alkali metal ion from the group of lithium, sodium, potassium, rubidium, caesium, preferably from the group of lithium, sodium, potassium, or an
alkaline earth metal ion from the group of beryllium, magnesium, calcium, strontium, barium, preferably from the group of magnesium and calcium,
heterocyclyl is oxetanyl, thiethanyl, tetrahydrofuryl and morpholinyl,
aryl is phenyl,
hetaryl (synonymous with heteroaryl, including as part of a larger unit such as, for example, hetarylalkyl) is a radical from the group of pyridyl, pyrimidyl, pyrazinyl, pyridazinyl, thiazolyl and pyrazolyl.

In the definitions which form the range of preference (4),

halogen is fluorine, chlorine, bromine and iodine, preferably in turn fluorine, chlorine and bromine.

Halogen-substituted radicals, for example haloalkyl, are mono- or polyhalogenated, up to the maximum number of possible substituents. In the case of polyhalogenation, the halogen atoms may be identical or different. In this case, halogen is fluorine, chlorine, bromine or iodine, especially fluorine, chlorine or bromine.

Saturated or unsaturated hydrocarbon radicals, such as alkyl or alkenyl, may each be straight-chain or branched if possible, including in combination with heteroatoms, as, for example, in alkoxy.

Unless stated otherwise, optionally substituted radicals may be mono- or polysubstituted, where the substituents in the case of poly substitutions may be the same or different.

When T in the A radical of the formula (A-a)

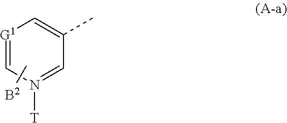

is an electron pair, the radical takes the form of the pyridine derivative of the formula

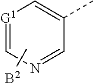

When T in the A radical of the formula (A-a)

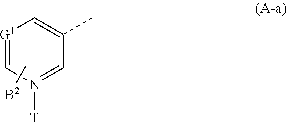

is oxygen, the radical takes the form of the pyridine N-oxide derivative of the formula

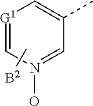

The representation of the formal charges (+at nitrogen and − at oxygen) was dispensed with here.

The radical definitions or elucidations given in general terms or listed within areas of preference apply correspondingly to end products and to starting materials and intermediates. These radical definitions can be combined with one another as desired, i.e. including combinations between the respective ranges of preference.

Preference is given in accordance with the invention to compounds of the formula (I) which contain a combination of the definitions listed above as being preferred (area of preference (1)).

Particular preference is given in accordance with the invention to compounds of the formula (I) which contain a combination of the definitions listed above as being particularly preferred (area of preference (2)).

Very particular preference is given in accordance with the invention to compounds of the formula (I) which contain a combination of the definitions listed above as being very particularly preferred (area of preference (3)).

Especial preference is given in accordance with the invention to compounds of the formula (I) which contain a combination of the definitions listed above as being particular definitions (area of preference (4)).

A preferred embodiment of the invention relates to compounds of the formula (I) in which A is the radical of the formula (A-a)

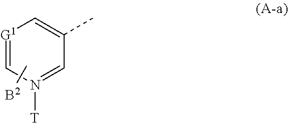

A further preferred embodiment of the invention relates to compounds of the formula (I) in which A is pyridin-3-yl.

A further preferred embodiment of the invention relates to compounds of the formula (I) in which A is 5-fluoropyridin-3-yl.

A further preferred embodiment of the invention relates to compounds of the formula (I) in which A is pyrimidin-5-yl.

A further preferred embodiment of the invention relates to compounds of the formula (I) in which A is pyridazin-4-yl.

A further preferred embodiment of the invention relates to compounds of the formula (I) in which R² has the definitions given under a).

A further preferred embodiment of the invention relates to compounds of the formula (I) in which R² has the definitions given under b).

A further preferred embodiment of the invention relates to compounds of the formula (I) in which R² has the definitions given under c).

A further preferred embodiment of the invention relates to compounds of the formula (I) in which R² has the definitions given under d).

A further preferred embodiment of the invention relates to compounds of the formula (I) in which R² has the definitions given under e).

A further preferred embodiment of the invention relates to compounds of the formula (I) in which R² has the definitions given under f).

A further preferred embodiment of the invention relates to compounds of the formula (I) in which R² is the (D-2) radical

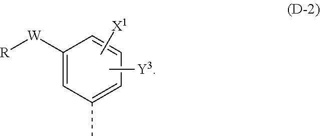

The radical definitions or elucidations given above in general terms or within preferred ranges apply correspondingly to the end products (including the compounds of the formulae (I-A) to (I-N) shown later), and to the starting materials and intermediates. These radical definitions can be combined with one another as desired, i.e. including combinations between the respective areas of preference.

In a preferred embodiment, the invention relates to compounds of the formula (I-A)

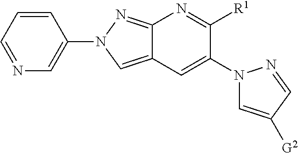

In a further preferred embodiment, the invention relates to compounds of the formula (I-B)

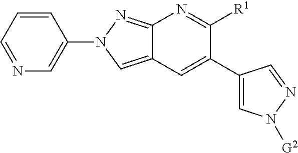

In a further preferred embodiment, the invention relates to compounds of the formula (I-C)

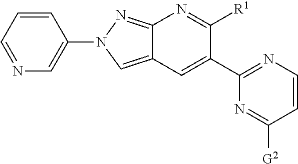

In a further preferred embodiment, the invention relates to compounds of the formula (I-D)

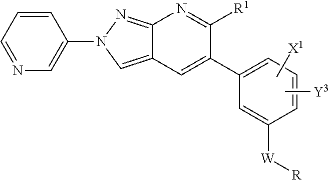

In a further preferred embodiment, the invention relates to compounds of the formula (I-E)

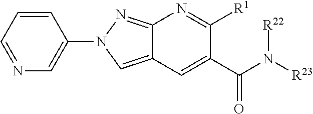

In a further preferred embodiment, the invention relates to compounds of the formula (I-F)

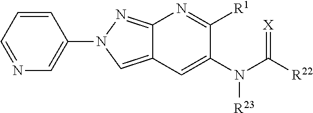

In a further preferred embodiment, the invention relates to compounds of the formula (I-G)

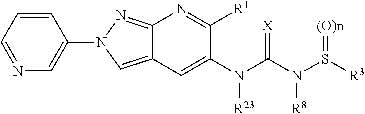

In a preferred embodiment, the invention relates to compounds of the formula (I-H)

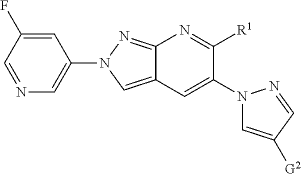

In a further preferred embodiment, the invention relates to compounds of the formula (I-I)

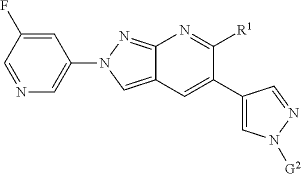

In a further preferred embodiment, the invention relates to compounds of the formula (I-J)

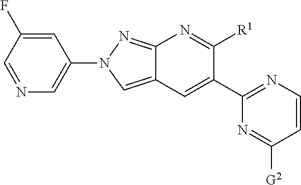

In a further preferred embodiment, the invention relates to compounds of the formula (I-K)

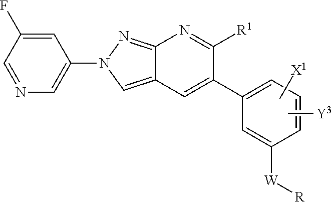

In a further preferred embodiment, the invention relates to compounds of the formula (I-L)

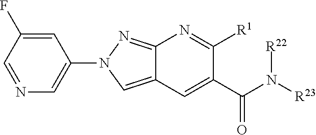

In a further preferred embodiment, the invention relates to compounds of the formula (I-M)

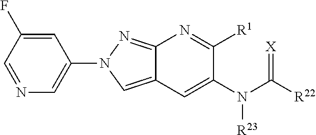

In a further preferred embodiment, the invention relates to compounds of the formula (I-N)

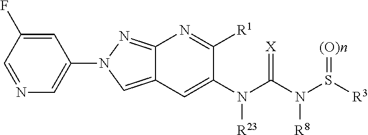

In the formulae (I-A) to (I-N), the variables G², R, R¹, R³, R⁸, R²², R²³ W, X¹, Y³ and n have the definitions given above.

The compounds of the formula (I) and their acid addition salts and metal salt complexes have good efficacy, especially for control of animal pests including arthropods and especially insects.

The compounds of the formula (I) may possibly also, depending on the nature of the substituents, be in the form of stereoisomers, i.e. in the form of geometric and/or optical isomers or isomer mixtures of varying compositions. This invention provides both the pure stereoisomers and any desired mixtures of these isomers, even though it is generally only compounds of the formula (I) that are discussed here.

The invention therefore relates both to the pure enantiomers and diastereomers and to mixtures thereof for controlling animal pests, including arthropods and particularly insects.

However, preference is given in accordance with the invention to using the optically active, stereoisomeric forms of the compounds of the formula (I) and salts thereof.

Suitable salts of the compounds of the formula (I) which may be mentioned are customary nontoxic salts, i.e. salts with appropriate bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and with inorganic amines, for example triethylammonium, dicyclohexylammonium, N,N′-dibenzylethylenediammonium, pyridinium, picolinium or ethanolammonium salts, salts with inorganic acids, for example hydrochlorides, hydrobromides, dihydrosulphates, trihydrosulphates, or phosphates, salts with organic carboxylic acids or organic sulphonic acids, for example formates, acetates, trifluoroacetates, maleates, tartrates, methane sulphonates, benzene sulphonate s or para-toluenesulphonates, salts with basic amino acids, for example arginates, aspartates or glutamates, and the like.

It has additionally been found that the compounds of the formula (I) and also those compounds listed in Table 1 that are not covered by the formula (I) can be prepared by the processes described hereinafter.

Compounds of the formula (I) in which the heterocycle A is optionally B² radical-substituted pyrimidin-5-yl (A-a; G′=N), pyridin-3-yl (A-a; =pyrazin-2-yl (A-b), pyridazin-3-yl (A-c), thiazol-5-yl (A-d), isothiazol-4-yl (A-e) and pyrazol-4-yl (A-f) can be prepared, for example, according to Reaction Scheme I in two steps.

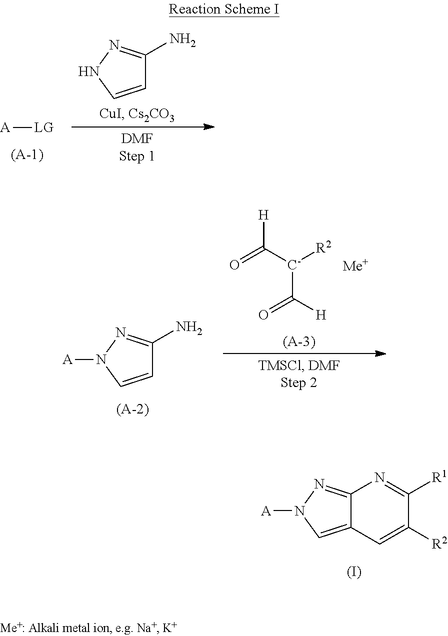

In Reaction Scheme I, A and R², unless stated otherwise, have the definitions given above; R¹ is hydrogen.

For example, the heterocycles of the formula (A-1) substituted by a suitable leaving group (LG=halogen) can be reacted in a first reaction step with the appropriate 3-aminopyrazole to give compounds of the formula (A-2) which are then cyclized in a second reaction step, for example in the presence of trimethylsilyl chloride (TMSCl) in N,N-dimethylformamide, to form the compounds (I) (cf. Preparation Example 1).

The compounds of the formula (A-1) are commercially available or can be obtained by preparation processes known in principle; cf., for example, when LG=halogen and when A=pyrazin-2-yl (A-b; B²═H; LG=iodine) (WO 2013/159064 A1); pyridazin-4-yl (A-c; B²═H; LG=iodine; A. Seggio et al., J. Org. Chem. 27, 6602-6605, 2007); thiazol-5-yl (A-d; B²═H; LG=bromine, iodine) and thiazol-4-yl (A-d; B²═H; LG=iodine; D. W. Brown et al., Science of Synthesis 11, 507-572, 2002); 1-methyl-1H-pyrazol-4-yl (A-f; B²═H, R³═CH₃; LG=iodine; WO 2014/022128 A1).

The compounds of the formula (A-2) are commercially available (A=3-pyridinyl/Aurora Building Blocks, A=5-pyrimidinyl/Otava Building Blocks; A=2-pyrazinyl/Aurora Building Blocks; A=1-methyl-1H-pyrazol-4-yl/UORSY Building Block Library) or can be obtained by preparation processes known in principle; cf., for example, for 1-(3-pyridinyl)-1H-pyrazol-3-amine (A-a; B²═H, G¹=C—H; T=electron pair; see also Preparation Example 1, step 1).

The compounds of the formula (A-3) are commercially available or can be obtained by preparation processes known in principle; cf., for example, for sodium 2-bromopropanediolate (R²=bromine, Me⁺═Na⁺; E. J. Corey et al., Tetrahedron Lett. 50, 4577-4580, 1976); sodium 2-nitropropanediolate hydrate (R²=nitro, Me⁺═Na⁺; cf. synthesis of intermediates in the Experimental).

Alternatively, the 2-halogen-substituted 3-pyridinecarboxaldehydes of the formula (A-4) can be converted in a first reaction step with sodium azide to the corresponding 2-azido-3-pyridinecarboxyaldehydes of the formula (A-5), which then react with 3-amino-substituted heterocycles of the formula (A-6) in a second reaction step to give compounds of the formula (A-7), called “Schiff bases”, and, in a third reaction step, cyclize under reflux temperature in a suitable solvent to form the compounds (I) (cf., for example, for 5-bromo-2-(3-pyridyppyrazolo[3,4-b]pyridine (A-a; B²═H, ═C—H; T=electron pair; see also Preparation Example 1, step 1; Reaction scheme II).

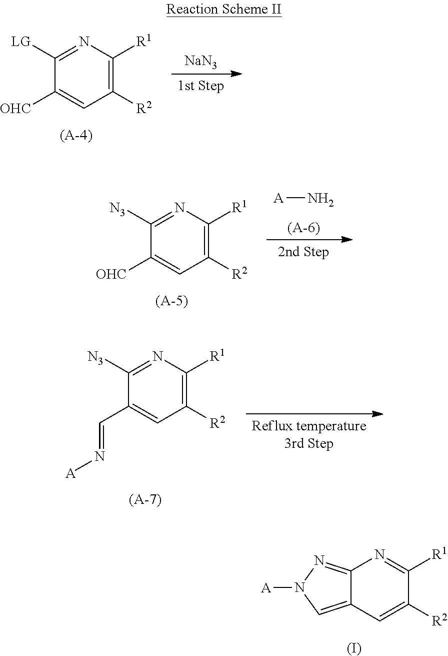

In Reaction Scheme II, A, R′ and R², unless stated otherwise, have the definitions given above.

Azomethine derivatives or “Schiff bases” of amino-substituted heterocycles have various applications (for example, they form metal complexes or are biologically active) and can be obtained by customary processes (cf. also V. Shama, et al., Intern. J. Univ. Pharm. Bio Science 2013, 2, 241-57 and literature cited therein).

The compounds of the formula (A-4) having a suitable leaving group (LG=halogen) are commercially available (for example when R¹═H, R²═CN, 6-fluoro-5-formylnicotinenitrile; R¹═H, R²═COOH, 6-fluoro-5-formylnicotic acid/Sunshine Chemlab Product List) or can be obtained by preparation processes known in principle (for example when R¹═Cl, R²═H, 6-chloro-2-fluoro-3-pyridinecarbaldehyde, WO 2012/078608 A1; R¹═H, R²═Cl, 5-chloro-2-fluoro-3-pyridinecarbaldehyde, WO 2012/083117 A1).

Alternatively, it is also possible to utilize varied reaction conditions for the cyclization, for example a metal-catalysed one-pot three-component synthesis (cf. M. J. Kumar et al. Org. Lett. 13, 3542-3545, 2011 and literature cited therein).

Compounds of the formula (I) in which R² is a radical from the group of (B-1) to (B-34) can be prepared by commonly known methods, for example from compounds of the formula (I) in which R² is preferably halogen from the group of bromine and iodine (Method A: cf. J. C. Antilla et al., J. Org. Chem., 2004, 69, 5578-5587 and Method B: cf. H. Dong et al., Org. Lett., 2011, 13, 2726 2729; Ch. O. Ndubaku et al., J. Med. Chem., 2013, 56, 4597 4610; T. Furuya et al., J. Am. Chem. Soc., 2010, 132, 3793-3807).

For example, the compounds of the formula (I) in which R² is a (B-2), (B-4) or (B-23) radical can be obtained according to Reaction Scheme III.

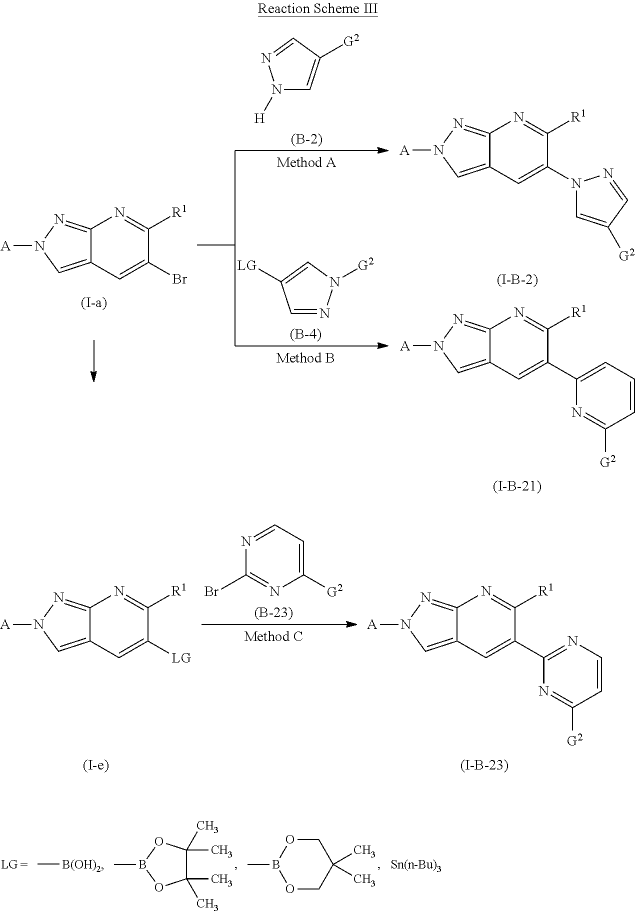

In Reaction Scheme III, the compounds (I-e) and (B-4) in which G² has the definition given above have a nucleofugic leaving group LG, optionally generated in situ (cf. also Preparation Example 8).

The preparation of compounds of the formula (I) in which R² is a (B-1), (B-2), (B-10), (B-29) or (B-30) radical is conducted analogously to Method A, which is known from the literature, preferably in the presence of copper(I) iodide and basic reaction auxiliaries, for example trans-N,N′-dimethylcyclohexane-1,2-diamine and potassium carbonate, in a suitable solvent or diluent. Useful solvents or diluents include all inert organic solvents, for example aliphatic or aromatic hydrocarbons.

Preference is given to using aromatic hydrocarbons, for example toluene.

The preparation of compounds of the formula (I) in which R² is a (B-3) to (B-9), (B-11) to (B-28) and (B-31) to (B-33) radical may be in accordance with Methods B and C shown in Reaction Scheme III.

For example, the compounds (B-3) to (B-9), (B-11) to (B-28) and (B-31) to (B-33) having a suitable leaving group (LG=B(OH)₂) or (hetero)arylboronic ester (LG=B(OR)₂) can be reacted with the appropriate compounds of the general formula (I-a) by known methods (cf. Chem. Rev. 1995, 95, 2457-2483; Tetrahedron 2002, 58, 9633-9695; Metal-Catalyzed Cross-Coupling Reactions (eds.: A. de Meijere, F. Diederich), 2^{nd} ed., Wiley-VCH, Weinheim, 2004; Ch. O. Ndubaku et al., J. Med. Chem., 2013, 56, 4597-4610) in the presence of suitable catalysts from the group of the transition metal salts to give compounds of the formula (I-B-3) to (I-B-9), (I-B-11) to (I-B-28) and (I-B-31) to (I-B-33).

Some of the compounds (B-3) to (B-9), (B-11) to (B-13) and (B-21) to (B-33) having a suitable leaving group (LG=B(OH)₂ or (hetero)arylboronic ester, LG=B(OR)₂) are known or can be prepared by commonly known methods: for example, 1-(methyl-1H-pyrazol-4-yl)boronic acid [(B-3), LG=B(OH)₂, G²=hydrogen, WO 2009/155527], 2-phenyl-4-(4,4,5,5-tetramethyl-1,3,2-dioxaborolan-2-yl)oxazole [(B-6), LG=B(OCMe₂)₂, G²=phenyl, WO 2010/094755]; thiazol-2-ylboronic acid [(B-7), LG=B(OH)₂, G²=hydrogen, U.S. Pat. No. 6,310,095 B1]; 5-phenyl-1,2,4-thiadiazol-3-ylboronic acid [(B-13), LG=B(OH)₂, G²=phenyl, DE 19710614 A1], pyridin-3-ylboronic acid [(B-21) vs (B-22), LG=B(OH)₂, G²=hydrogen, WO 2013/186089]; 1,3,5-triazin-2-ylboronic acid [(B-28), LG=B(OH)₂, G²=hydrogen, KR 2011/079401].

Alternatively, the compounds of the formula (I-a) can first be converted by methods known from the literature to compounds of the formula (I-e) which then react further with halogen-activated heterocycles according to Reaction Scheme III by Method C (cf. T. Ishiyama et al., J. Org. Chem., 1995, 60, 7508 7510; WO 2010/151601).

Some halogen-activated compounds (B-3) to (B-9), (B-11) to (B-13) and (B-21) to (B-33) are known, and/or they can be prepared by commonly known methods: for example, 3-bromo-4,5-dihydro-1-phenyl-1H-pyrazole [(B-18), LG=Br, G²=phenyl, J. Elguero et al., Bull. Soc. Chim. France 1996, 5, 1683-1686].

The preparation of compounds of the formula (I) in which R² is a (B-21) or (B-23) radical can be conducted in accordance with Methods B and C, which are known from the literature and shown in Reaction Scheme III, preferably in the presence of suitable coupling catalysts, basic reaction auxiliaries and in a suitable solvent or diluent. Useful solvents or diluents include all inert organic solvents, for example aliphatic or aromatic hydrocarbons.

Preference is given to using aromatic hydrocarbons, for example toluene.

The preparation of compounds of the formula (I) in which R² is a (B-3) to (B-9), (B-11) to (B-28) and (B-31) to (B-33) radical can be conducted in accordance with Methods B and C, which are shown in Reaction Scheme III, preferably in the presence of suitable coupling catalysts, basic reaction auxiliaries and in a suitable solvent or diluent.

Examples of preferred coupling catalysts include palladium catalysts such as [1,1′-bis(diphenylphosphino)ferrocene]dichloropalladium(II) or tetrakis(triphenylphosphine)palladium.

Suitable basic reaction auxiliaries used for conducting the processes according to Reaction Scheme III are preferably carbonates of sodium or potassium.

Preference is given to using nitriles such as acetonitrile, benzonitrile, especially acetonitrile, or ethers such as diethyl ether, dioxane, tetrahydrofuran, 1,2-dimethoxyethane, especially 1,2-dimethoxyethane in combination with water.

Compounds of the formula (I) in which R² is a radical from the group of (C-1) to (C-9) or is CX—NR²²R²³ can be prepared by commonly known methods, for example from compounds of the formula (I) in which R² is a carboxyl group, following suitable activation (i.e. LG is a nucleofugic leaving group, optionally generated in situ).

For example, the compounds of the formula (I) in which R² is a (C-1) radical or is —CX—NR²²R²³ can be obtained according to Reaction Scheme IV.

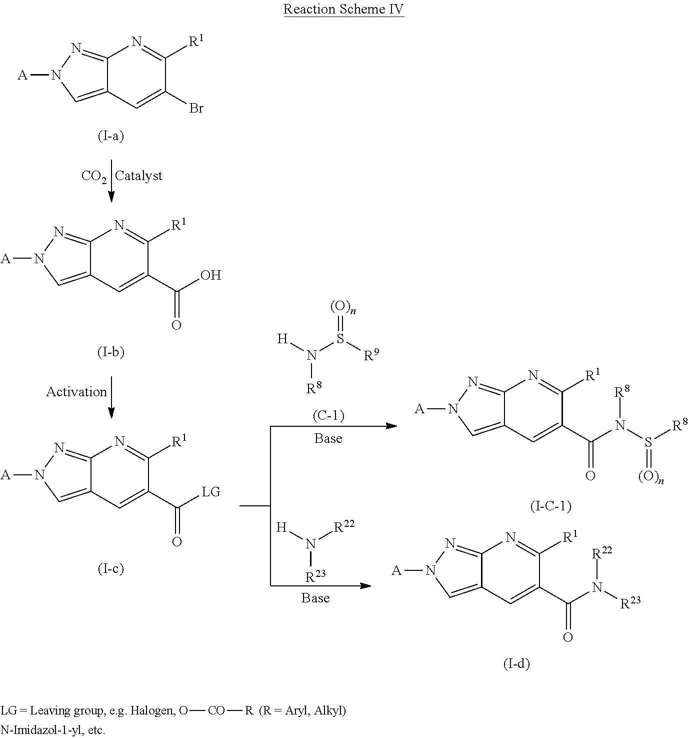

A suitable route for the compounds of the formula (I) in which R² is carboxyl may be the N-substituted 5-bromopyrazolo[3,4-b]pyridines (A-1; R²═Br) preparable according to Reaction Scheme I (cf. I-a). The compounds of the formula (I-a) can be converted here to compounds of the formula (I-b) by a palladium-catalysed hydroxycarbonylation (cf. S. Korsager et al., J. Amerc. Chem Soc. 135, 2891-2894, 2013 and the review articles cited therein).

Useful condensing agents for activation of the carboxylic acids of the formula (I-b) include all condensing agents customarily usable for such amidation reactions. Examples include acid halide formers such as phosgene, phosgene derivatives such as carbonyldiimidazole (CDI), phosphorus trichloride, oxalyl chloride or thionyl chloride; carbodiimides such as N,N′-dicyclohexylcarbodiimide (DCC) and 1-(3-dimethylaminopropyl)-3-ethylcarbodiimide (EDCI), or other customary condensing agents such as phosphorus pentoxide, polyphosphoric acid, N,N′-carbonyldiimidazole, 2-chloropyridine 1-methoiodide (Mukaiyama's reagent), 2-ethoxy-N-ethoxycarbonyl-1,2-dihydroquinoline (EEDQ), triphenylphosphine/carbon tetrachloride, bromotripyrrolidinophosphonium hexafluorophosphate (BROP), O-(1H-benzotriazol-1-yloxy)tris(dimethylamino)phosphonium hexafluorophosphate (BOP), bis(2-oxo-3-oxazolidinyl)phosphinic chloride (BOP—Cl), N,N,N′,N′-bis(tetramethylene)chlorouronium tetrafluoroborate, O-(1H-benzotriazol-1-yl)-N,N,N′,N′-tetramethyluronium hexafluorophosphate (HBTU), O-(1H-benzotriazol-1-yl)-N,N,N′,N′-bis(tetramethylene)uronium hexafluorophosphate, O-(1H-benzotriazol-1-yl)-N,N, N′,N′-tetramethyluronium tetrafluoroborate (TBTU), O-(1H-benzotriazol-1-yl)-N,N,N′,N′-bis(tetramethylene)uronium tetrafluoroborate, O-(7-azabenzotriazol-1-yl)-N, N,N′,N′-tetramethyluronium hexafluorophosphate (HATU), 1-hydroxybenzotriazole (HOBt) and 4-(4,6-dimethoxy-1,3,5-triazin-2-yl)-4-methylmorpholinium salt (DMT.MM), usually available as the chloride. These reagents can be used separately or, if appropriate, in combination.

For controlled activation of the compounds of the formula (I-b), an alternative option is to use mixed anhydrides (LG=COOR), which lead to the preparation of compounds of the formulae (I-C-1) and (I-d) (cf. G. W. Anderson et al. J. Am. Chem. Soc. 1967, 89, 5012-5017). Various chloroformic esters can be used in this process, for example isobutyl chloroformate (LG=COOR where R=isobutyl) and isopropyl chloroformate (LG=COOR where R=isopropyl). It is likewise possible for this purpose to use diethylacetyl chloride, trimethylacetyl chloride and similar compounds.

The subsequent reactions of the activated compounds of the formula (I-c) with the respective amine components according to Reaction Scheme IV are optionally conducted in the presence of a suitable reaction auxiliary and in the presence of a suitable solvent or diluent.

Suitable reaction auxiliaries for conducting the processes according to Reaction Scheme IV are basic reaction auxiliaries.

Examples include the hydroxides, hydrides, oxides and carbonates of lithium, sodium, potassium, magnesium, calcium and barium, and also further basic compounds such as amidine bases or guanidine bases, such as 7-methyl-1,5, 7-triazabicyclo[4.4.0]dec-5-ene (MTBD); diazabicyclo[4.3.0]nonene (DBN), diazabicyclo[2.2.2]octane (DABCO), 1,8-diazabicyclo[5.4.0]undecene (DBU), cyclohexyltetrabutylguanidine (CyTBG), cyclohexyltetramethylguanidine (CyTMG), N,N,N,N-tetramethyl-1,8-naphthalenediamine, pentamethylpiperidine, tertiary amines, such as triethylamine, trimethylamine, tribenzylamine, triisopropylamine, tributylamine, tricyclohexylamine, triamylamine, trihexylamine, N, N-dimethylaniline, N,N-dimethyltoluidine, N,N-dimethyl-p-aminopyridine, N-methylpyrrolidine, N-methylpiperidine, N-methylimidazole, N-methylpyrazole, N-methylmorpholine, N-methylhexamethylenediamine, pyridine, 4-pyrrolidinopyridine, 4-dimethylaminopyridine, quinoline, α-picoline, β-picoline, isoquinoline, pyrimidine, acridine, N,N,N′,N′-tetramethylenediamine, N,N′,N′-tetraethylenediamine, quinoxaline, N-propyldiisopropylamine, N-ethyldiisopropylamine (Hünig's base), N,N′-dimethylcyclohexylamine, 2,6-lutidine, 2,4-lutidine or triethylenediamine

Basic reaction auxiliaries usable for conducting the processes according to Reaction Scheme IV may be any suitable acid binders, for example amines, especially tertiary amines, and alkali metal and alkaline earth metal compounds.

The compounds of the formula (I-C-1) or (I-d) are preferably prepared using tertiary amines such as N-propyldiisopropylamine or N-ethyldiisopropylamine (DIEA; Htinig's base).

Suitable solvents or diluents include all inert organic solvents, for example aliphatic or aromatic hydrocarbons (such as petroleum ether, toluene), halogenated hydrocarbons (such as chlorotoluene, dichloromethane, chloroform, 1,2-dichloroethane), ethers (such as diethyl ether, dioxane, tetrahydrofuran, 1,2-dimethoxyethane), esters (such as ethyl or methyl acetate), nitrohydrocarbons (such as nitromethane, nitroethane, nitrobenzene), nitriles (such as acetonitrile, benzonitrile), amides (such as N,N-dimethylformamide, N,N-dimethylacetamide, N-methylformanilide, N-methylpyrrolidone, hexamethylphosphoramide), and also dimethyl sulphoxide or water or mixtures of the solvents mentioned.

Preference is given to using amides as solvents, for example N,N-dimethylformamide.

Compounds of the formula (I) in which R² is a radical from the group of (D-1) to (D-3) can be prepared by known methods, for example from compounds of the formula (I) in which R² is bromine or iodine.

For example, the compounds of the formula (I) in which R² is a radical from the group of (D-1) to (D-3) can be obtained according to Reaction Scheme V by methods known from the literature (cf. US2013/0267493; T. Furuya et al., J. Am. Chem. Soc., 2010, 132, 3793-3807).

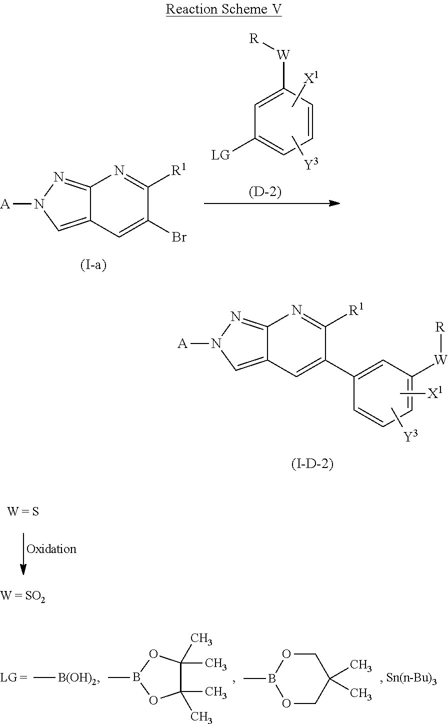

In Reaction Scheme V, the compounds (D-2) in which W is S, SO or SO₂ have a nucleofugic leaving group LG, optionally generated in situ.

For example, the compounds (D-1) to (D-3) having a suitable leaving group (LG=B(OH)₂) or (hetero)arylboronic ester (LG=B(OR)₂) can be reacted with the appropriate compounds of the formula (I-a) according to known methods (cf. Chem. Rev. 1995, 95, 2457-2483; Tetrahedron 2002, 58, 9633-9695; Metal-Catalyzed Cross-Coupling Reactions (Eds.: A. de Meijere, F. Diederich), 2nd ed., Wiley-VCH, Weinheim, 2004) in the presence of suitable catalysts from the group of the transition metal salts to give compounds of the formula (I-D-1) to (I-D-3).

The preparation of compounds of the formula (I) in which R² is a (D-1) to (D-3) radical can be conducted in accordance with Reaction Scheme V, preferably in the presence of suitable coupling catalysts and basic reaction auxiliaries, and in a suitable solvent or diluent.

Examples of preferred coupling catalysts include palladium catalysts such as [1,1′-bis(diphenylphosphino)ferrocene]dichloropalladium(II) or tetrakis(triphenylphosphine)palladium.

Suitable basic reaction auxiliaries used for conducting the processes according to Reaction Scheme III are preferably carbonates of sodium or potassium.

Preference is given to using nitriles such as acetonitrile, benzonitrile, especially acetonitrile, or ethers such as diethyl ether, dioxane, tetrahydrofuran, 1,2-dimethoxyethane, especially 1,2-dimethoxyethane in combination with water (cf. also Preparation Examples 2 and 3).

The subsequent oxidation of the sulphur in the compounds of the formula (I-D-2) in which W is sulphur leads to compounds of the formula (I-D-2) in which W is SO or SO₂ (cf. Reaction Scheme V; see Preparation Examples 3 and 5 for (I-D-2)).

Compounds of the formula (I) in which W is SO (sulphoxides) or SO₂ (sulphones) can be prepared from compounds of the formula (I) in which W is S (thioethers) by oxidation by processes known from the literature, for example by means of an oxidizing agent in a suitable solvent or diluent. Suitable oxidizing agents are, for example, dilute nitric acid, hydrogen peroxide, Oxone® and peroxycarboxylic acids, for example meta-chloroperbenzoic acid. Suitable solvents or diluents are inert organic solvents, typically acetonitrile and halogenated solvents such as dichloromethane, chloroform or dichloroethane, and water and alcohols such as methanol for the reaction with Oxone®.

A variety of methods are suitable for production of enantiomerically enriched sulphoxides, as described by G. E. O'Mahony et al., in ARKIVOC (Gainesville, Fla., United States), 2011, 1, 1-110: metal-catalysed asymmetric oxidations of thioethers, for example with titanium or vanadium as the most frequently utilized catalyst sources, in the form of Ti(OⁱPr₄) or VO(acac)₂, together with a chiral ligand and an oxidizing agent such as tert-butyl hydroperoxide (TBHP), 2-phenylpropan-2-yl hydroperoxide (CHP) or hydrogen peroxide; non-metal-catalysed asymmetric oxidations through use of chiral oxidizing agents or chiral catalysts; electrochemical or biological asymmetric oxidations and also kinetic resolution of sulphoxides and nucleophilic displacement (by Andersen's method).

Compounds of the formula (I) in which R² is —NR²³—CX—R²² can be obtained, for example, from compounds of the formula (I) in which R² is a group of the formula —NHR²³ via N-acylation reaction using activated compounds of the formula LG-CX—R²² in which LG is a nucleofugic leaving group, optionally generated in situ.

These compounds of the formula (I) in which R² is —NHR²³ can be prepared by known methods from compounds of the formula (I) in which R² is a nitro group (cf. I-f) by reduction in compounds of the formula (I) in which R² is an amino group (cf. I-g), according to Reaction Scheme VI (cf. also Preparation Example 1; step 4 for A=3-pyridinyl, ═H).

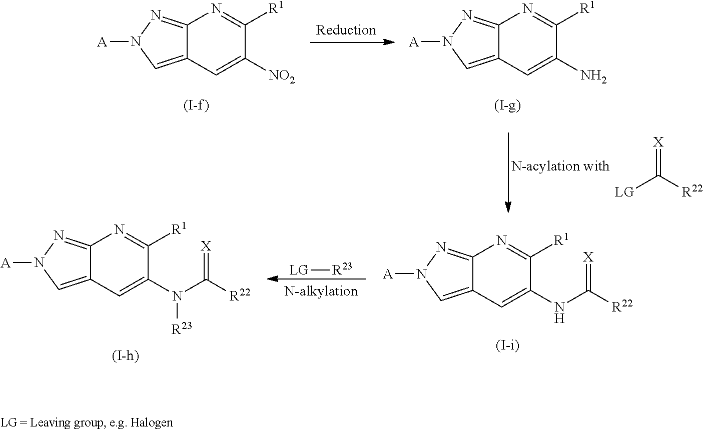

The compounds of the formula (I-i) can be obtained by N-acylation from the compounds of the formula (I j) (cf. Preparation Examples 7 and 8).

The compounds of the formula (I-h) can be prepared from the compounds (I-i), for example, by subsequent N-alkylation.

Alternatively, the compounds of the formula (I) in which R² is —NHR²³ can be prepared by commonly known methods from compounds of the formula (I) in which R² is a carboxyl group, according to Reaction Scheme VII.

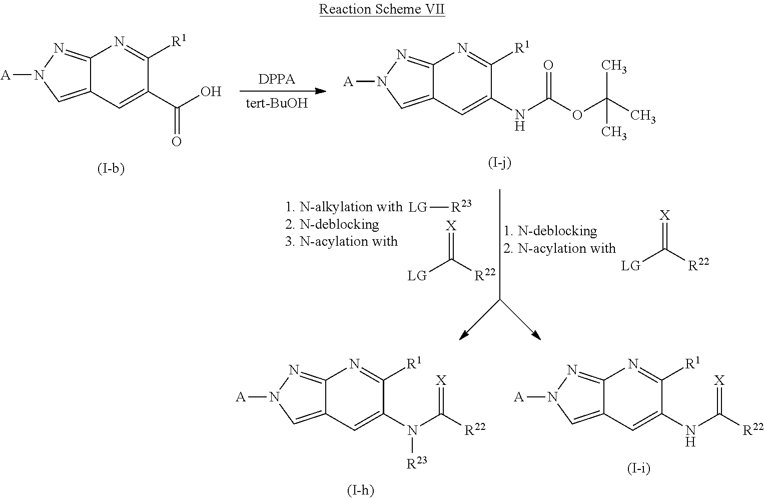

LG=Leaving group, e.g. Halogen

DPPA=diphenylphosphoryl azide

For example, compounds of the formula (I-f) can be obtained by Curtius degradation as described, for example, in Houben-Weyl, Methoden der Organischen Chemie [Methods of Organic Chemistry], Volume XI/1 (Georg Thieme Verlag Stuttgart), p. 865.

In this case, the compounds of the formula (I-b) can react, for example, directly with diphenylphosphoryl azide (DPPA) in the presence of tert-butanol to give compounds of the formula (I-j).

In general, for the removal of the protecting group, it is possible to use acidic or basic reaction auxiliaries according to the literature procedure. When protecting groups of the carbamate type are used, preference is given to using acidic reaction auxiliaries. When the tert-butyl carbamate protective group (Boc group) is used, for example, mixtures of mineral acids such as hydrochloric acid, hydrobromic acid, nitric acid, sulphuric acid, phosphoric acid or of organic acids such as benzoic acid, formic acid, acetic acid, trifluoroacetic acid, methanesulphonic acid, benzenesulphonic acid or toluenesulphonic acid, in a suitable diluent such as water and/or an organic solvent such as tetrahydrofuran, dioxane, dichloromethane, chloroform, ethyl acetate, ethanol or methanol are used. Preference is given to mixtures of hydrochloric acid or acetic acid with water and/or an organic solvent such as ethyl acetate.

When reference is made hereinafter to compounds of the formula (I), this also includes those compounds in Table 1 that are not covered by formula (I).

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention therefore encompasses pure stereoisomers and any desired mixtures of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. This preferably excludes methods for surgical or therapeutic treatment of the human or animal body and diagnostic methods carried out on the human or animal body.

The invention further relates to the use of the compounds of the formula (I) as pesticides, especially crop protection agents.

In the context of the present application, the term “pesticide” also always encompasses the term “crop protection agent”.

The compounds of the formula (I), given good plant tolerance, favourable homeotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, nematodes and molluscs, which are encountered in agriculture, in horticulture, in animal husbandry, in aquatic cultures, in forests, in gardens and leisure facilities, in the protection of stored products and of materials, and in the hygiene sector. They can preferably be used as pesticides. They are active against normally sensitive and resistant species and also against all or specific stages of development. The abovementioned pests include:

pests from the phylum of the Arthropoda, especially from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coffeae, Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;

from the class of the Chilopoda, for example Geophilus spp., Scutigera spp.;

from the order or the class of the Collembola, for example Onychiurus armatus; Sminthurus viridis;

from the class of the Diplopoda, for example Blaniulus guttulatus;

from the class of the Insecta, for example from the order of the Blattodea, for example Blatta orientalis, Blattella asahinai, Blattella germanica, Leucophaea maderae, Loboptera decipiens, Neostylopyga rhombifolia, Panchlora spp., Parcoblatta spp., Periplaneta spp., for example Periplaneta americana, Periplaneta australasiae, Pycnoscelus surinamensis, Supella longipalpa;

from the order of the Coleoptera, for example Acalymma vittatum, Acanthoscelides obtectus, Adoretus spp., Aethina tumida, Agelastica alni, Agriotes spp., for example Agriotes linneatus, Agriotes mancus, Alphitobius diaperinus, Amphimallon solstitialis, Anobium punctatum, Anoplophora spp., Anthonomus spp., for example Anthonomus grandis, Anthrenus spp., Apion spp., Apogonia spp., Atomaria spp., for example Atomaria linearis, Attagenus spp., Baris caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., for example Leucoptera coffeella, Lissorhoptrus oryzophilus, Listronotus (=Hyperodes) spp., Lixus spp., Luperomorpha xanthodera, Luperodes spp., Lyctus spp., Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oulema spp., for example Oulema melanopus, Oulema oryzae, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Sinoxylon perforans, Sitophilus spp., for example Sitophilus granarius, Sitophilus linearis, Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides;

from the order of the Dermaptera, for example Anisolabis maritime, Forficula auricularia, Labidura riparia;

from the order of the Diptera, for example Aedes spp., for example Aedes aegypti, Aedes albopictus, Aedes sticticus, Aedes vexans, Agromyza spp., for example Agromyza frontella, Agromyza parvicornis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae, Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterborniella subcincta, Pegomya spp., for example Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex, Toxotrypana curvicauda;

from the order of the Hemiptera for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inornata, Aphanostigma piri, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., for example Cacopsylla pyricola, Calligypona marginata, Capulinia spp., Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium corni (=Parthenolecanium corni), Lepidosaphes spp., for example Lepidosaphes ulmi, Lipaphis erysimi, Lopholeucaspis japonica, Lycorma delicatula, Macrosiphum spp., for example Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Neomaskellia spp., Nephotettix spp., for example Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Perkinsiella spp., Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus viburni, Psyllopsis spp., Psylla spp., for example Psylla buxi, Psylla mali, Psylla pyri, Pteromalus spp., Pulvinaria spp., Pyrilla spp., Quadraspidiotus spp., for example Quadraspidiotus juglansregiae, Quadraspidiotus ostreaeformis, Quadraspidiotus perniciosus, Quesada gigas, Rastrococcus spp., Rhopalosiphum spp., for example Rhopalosiphum maidis, Rhopalosiphum oxy acanthae, Rhopalosiphum padi, Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;

from the suborder of the Heteroptera, for example Aelia spp., Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., for example Lygocoris pabulinus, Lygus spp., for example Lygus elisus, Lygus hesperus, Lygus lineolaris, Macropes excavatus, Megacopta cribraria, Miridae, Monalonion atratum, Nezara spp., for example Nezara viridula, Nysius spp., Oebalus spp., Pentomidae, Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;

from the order of the Hymenoptera, for example Acromyrmex spp., Athalia spp., for example Athalia rosae, Atta spp., Camponotus spp., Dolichovespula spp., Diprion spp., for example Diprion similis, Hoplocampa spp., for example Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., Solenopsis invicta, Tapinoma spp., Technomyrmex albipes, Urocerus spp., Vespa spp., for example Vespa crabro, Wasmannia auropunctata, Xeris spp.;

from the order of the Isopoda, for example Armadillidium vulgare, Oniscus asellus, Porcellio scaber;

from the order of the Isoptera, for example Coptotermes spp., for example Coptotermes formosanus, Cornitermes cumulans, Cryptotermes spp., Incisitermes spp., Kalotermes spp., Microtermes obesi, Nasutitermes spp., Odontotermes spp., Reticulitermes spp., for example Reticulitermes flavipes, Reticulitermes hesperus;

from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diparopsis spp., Diatraea saccharalis, Earias spp., Ecdytolopha aurantium, Elasmopalpus lignosellus, Eldana saccharina, Ephestia spp., for example Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens, Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., for example Ostrinia nubilalis, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Prays spp., Prodenia spp., Protoparce spp., Pseudaletia spp., for example Pseudaletia unipuncta, Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.;

from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria;

from the order of the Phthiraptera, for example Damalinia spp., Haematopinus spp., Linognathus spp., Pediculus spp., Phylloxera vastatrix, Phthirus pubis, Trichodectes spp.;

from the order of the Psocoptera, for example Lepinotus spp., Liposcelis spp.;

from the order of the Siphonaptera, for example Ceratophyllus spp., Ctenocephalides spp., for example Ctenocephalides canis, Ctenocephalides felis, Pulex irritans, Tunga penetrans, Xenopsylla cheopis;

from the order of the Thysanoptera, for example Anaphothrips obscurus, Baliothrips biformis, Chaetanaphothrips leeuweni, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., for example Thrips palmi, Thrips tabaci;

from the order of the Zygentoma (=Thysanura), for example Ctenolepisma spp., Lepisma saccharina, Lepismodes inquilinus, Thermobia domestica;

from the class of the Symphyla, for example Scutigerella spp., for example Scutigerella immaculata;

pests from the phylum of the Mollusca, in particular from the class of the Bivalvia, for example Dreissena spp.;

and also from the class of the Gastropoda, for example Anion spp., for example Anion ater rufus, Biomphalaria spp., Bulinus spp., Deroceras spp., for example Deroceras laeve, Galba spp., Lymnaea spp., Oncomelania spp., Pomacea spp., Succinea spp.;

animal and human parasites from the phyla of the Platyhelminthes and Nematoda, for example Aelurostrongylus spp., Amidostomum spp., Ancylostoma spp, for example Ancylostoma duodenale, Ancylostoma ceylanicum, Acylostoma braziliensis, Angiostrongylus spp., Anisakis spp., Anoplocephala spp., Ascaris spp., Ascaridia spp., Baylisascaris spp., Brugia spp., for example Brugia malayi, Brugia timori, Bunostomum spp., Capillaria spp., Chabertia spp., Clonorchis spp., Cooperia spp., Crenosoma spp., Cyathostoma spp., Dicrocoelium spp., Dictyocaulus spp., for example Dictyocaulus filaria, Diphyllobothrium spp., for example Diphyllobothrium latum, Dipylidium spp., Dirofilaria spp., Dracunculus spp., for example Dracunculus medinensis, Echinococcus spp., for example Echinococcus granulosus, Echinococcus multilocularis, Echinostoma spp., Enterobius spp., for example Enterobius vermicularis, Eucoleus spp., Fasciola spp., Fascioloides spp., Fasciolopsis spp., Filaroides spp., Gongylonema spp., Gyrodactylus spp., Habronema spp., Haemonchus spp., Heligmosomoides spp., Heterakis spp., Hymenolepis spp., for example Hymenolepis nana, Hyostrongylus spp., Litomosoides spp., Loa spp., for example Loa Loa, Metastrongylus spp., Metorchis spp., Mesocestoides spp., Moniezia spp., Muellerius spp., Necator spp., Nematodirus spp., Nippostrongylus spp., Oesophagostomum spp., Ollulanus spp., Onchocerca spp, for example Onchocerca volvulus, Opisthorchis spp., Oslerus spp., Ostertagia spp., Oxyuris spp., Paracapillaria spp., Parafilaria spp., Paragonimus spp., Paramphistomum spp., Paranoplocephala spp., Parascaris spp., Passalurus spp., Protostrongylus spp., Schistosoma spp., Setaria spp., Spirocerca spp., Stephanofilaria spp., Stephanurus spp., Strongyloides spp., for example Strongyloides fuelleborni, Strongyloides stercoralis, Strongylus spp., Syngamus spp., Taenia spp., for example Taenia saginata, Taenia solium, Teladorsagia spp., Thelazia spp., Toxascaris spp., Toxocara spp., Trichinella spp., for example Trichinella spiralis, Trichinella nativa, Trichinella britovi, Trichinella nelsoni, Trichinella pseudopsiralis, Trichobilharzia spp., Trichostrongylus spp., Trichuris spp., for example Trichuris trichiura, Uncinaria spp., Wuchereria spp., for example Wuchereria bancrofti;

plant pests from the phylum of the Nematoda, i.e. phytoparasitic nematodes, especially Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hirschmaniella spp., Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., for example Paratrichodorus minor, Paratylenchus spp., Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.

In addition, it is possible to control, from the sub-kingdom of the Protozoa, the order of the Coccidia, for example Eimeria spp.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). They can, as the case may be, also be used as intermediates or precursors for the synthesis of other active ingredients.

### Formulations

The present invention further relates to formulations and use forms prepared therefrom as pesticides, for example drench, drip and spray liquors, comprising at least one compound of the formula (I). Optionally, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further agrochemically active ingredients.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known way, for example by mixing the compounds of the formula (I) with auxiliaries such as, for example, extenders, solvents and/or solid carriers and/or other auxiliaries such as, for example, surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the simple and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and water.

In principle, it is possible to use all suitable solvents. Examples of suitable solvents are aromatic hydrocarbons, such as xylene, toluene or alkylnaphthalenes, chlorinated aromatic or aliphatic hydrocarbons, such as chlorobenzene, chloroethylene or methylene chloride, aliphatic hydrocarbons, such as cyclohexane, paraffins, mineral oil fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, and also water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic minerals such as finely divided silica, alumina and natural or synthetic silicates, resins, waxes and/or solid fertilizers. It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, maize cobs and tobacco stalks.

It is also possible to use liquefied gaseous extenders or solvents. Especially suitable are those extenders or carriers which are gaseous at standard temperature and under atmospheric pressure, for example aerosol propellants such as halogenated hydrocarbons, and also butane, propane, nitrogen and carbon dioxide.

Examples of emulsifiers and/or foam formers, dispersants or wetting agents having ionic or nonionic properties or mixtures of these surface-active substances are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and if the application takes place in water.

Further auxiliaries which may be present in the formulations and the use forms derived therefrom are dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components which may be present are stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

It is possible if appropriate for still further auxiliaries to be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce dynamic surface tension, for example dioctyl sulphosuccinate, or increase viscoelasticity, for example hydroxypropylguar polymers.

Useful penetrants in the present context are all those substances which are typically used to improve the penetration of active agrochemical ingredients into plants. Penetrants are defined in this context by their ability to penetrate from the (generally aqueous) application liquor and/or from the spray coating into the cuticle of the plant and hence increase the mobility of the active ingredients in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

### Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compounds of the formula (I) may be present in a mixture with other active ingredients or semiochemicals such as attractants and/or bird repellents and/or plant activators and/or growth regulators and/or fertilizers. Likewise, the compounds of the formula (I) can be used in mixtures with agents to improve plant properties, for example growth, yield and quality of the harvested material.

In a particular embodiment according to the invention, the compounds of the formula (I) are present in formulations or in the use forms prepared from these formulations in a mixture with further compounds, preferably those as described below.

If one of the compounds mentioned below can occur in different tautomeric forms, these forms are also included even if not explicitly mentioned in each case.

### Insecticides/Acaricides/Nematicides

The active ingredients specified here with their common names are known and are described for example in “The Pesticide Manual”, 16th ed., British Crop Protection Council 2012, or can be searched for on the Internet (e.g. http://www.alanwood.net/pesticides).

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e. g. acephate, azamethiphos, azinphos-ethyl, azinphos-methyl, cadusafos, chlorethoxyfos, chlorfenvinphos, chlormephos, chloropyrifos, chloropyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel antagonists, for example cyclodiene-organochlorines, e.g. chlordane and endosulphan or phenylpyrazoles (fiproles), e.g. ethiprole and fipronil.

(3) Sodium channel modulators/voltage-gated sodium channel blockers, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomers)], tralomethrin and transfluthrin or DDT or methoxychlor.

(4) Nicotinergic acetylcholine receptor (nAChR) agonists, for example neonicotinoids, e.g. acetamiprid, clothianidin, dinotefuran, imidacloprid, nitenpyram, thiacloprid and thiamethoxam or nicotine or sulphoxaflor or flupyradifurone.

(5) Allosteric activators of the nicotinergic acetylcholine receptor (nAChR), for example spinosyns, e.g. spinetoram and spinosad.

(6) Chloride channel activators, for example avermectins/milbemycins, e.g. abamectin, emamectin benzoate, lepimectin and milbemectin.

(7) Juvenile hormone imitators, for example, juvenile hormone analogues, e.g. hydroprene, kinoprene and methoprene or fenoxycarb or pyriproxyfen.

(8) Active compounds having unknown or nonspecific mechanisms of action, for example alkyl halides, e.g. methyl bromide and other alkyl halides; or chloropicrine or sulphuryl fluoride or borax or tartar emetic.

(9) Selective antifeedants, e.g. pymetrozine or flonicamid.

(10) Mite growth inhibitors, e.g. clofentezine, hexythiazox and diflovidazin or etoxazole.

(11) Microbial disruptors of the insect gut membrane, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.

(12) Oxidative phosphorylation inhibitors, ATP disruptors, for example diafenthiuron or organotin compounds, e.g. azocyclotin, cyhexatin and fenbutatin oxide or propargite or tetradifon.

(13) Oxidative phosphorylation decouplers that interrupt the H proton gradient, for example chlorfenapyr, DNOC and sulphluramid.

(14) Nicotinergic acetylcholine receptor antagonists, for example bensultap, cartap hydrochloride, thiocyclam, and thiosultap-sodium.

(15) Inhibitors of chitin biosynthesis, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, for example buprofezin.

(17) Moulting inhibitors (especially for Diptera, i.e. dipterans), for example cyromazine.

(18) Ecdysone receptor agonists, for example chromafenozide, halofenozide, methoxyfenozide and tebufenozide.

(19) Octopaminergic agonists, for example amitraz.

(20) Complex-III electron transport inhibitors, for example hydramethylnon or acequinocyl or fluacrypyrim.

(21) Complex-I electron transport inhibitors, for example METI acaricides, e.g. fenazaquin, fenpyroximate, pyrimidifen, pyridaben, tebufenpyrad and tolfenpyrad or rotenone (Derris).

(22) Voltage-gated sodium channel blockers, for example indoxacarb or metaflumizone.

(23) Inhibitors of acetyl-CoA carboxylase, for example tetronic and tetramic acid derivatives, e.g. spirodiclofen, spiromesifen and spirotetramat.

(24) Complex-IV electron transport inhibitors, for example phosphines, e.g. aluminium phosphide, calcium phosphide, phosphine and zinc phosphide or cyanide.

(25) Complex-II electron transport inhibitors, for example cyenopyrafen and cyflumetofen.

(28) Ryanodine receptor effectors, for example diamides, e.g. chlorantraniliprole, cyantraniliprole and flubendiamide.

Further active ingredients having an unknown or unclear mechanism of action, for example afidopyropen, afoxolaner, azadirachtin, benclothiaz, benzoximate, bifenazate, bromopropylate, chinomethionat, cryolite, cyclaniliprole, cycloxaprid, cyhalodiamide, dicloromezotiaz, dicofol, diflovidazin, flometoquin, fluensulfone, flufenerim, flufenoxystrobin, flufiprole, fluhexafon, fluopyram, fluralaner, fufenozide, guadipyr, heptafluthrin, imidaclothiz, iprodione, meperfluthrin, paichongding, pyflubumide, pyridalyl, pyrifluquinazon, pyriminostrobin, tetramethylfluthrin, tetraniliprole, tetrachlorantraniliprole, tioxazafen, triflumezopyrim and iodomethane; and additionally preparations based on Bacillus firmus (1-1582, BioNeem, Votivo), and the following known active compounds: 1-{2-fluoro-4-methyl-5-[(2,2,2-trifluoroethypsulphinyl]phenyl}-3-(trifluoromethyl)-1H-1,2,4-triazole-5-amine (known from WO2006/043635), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-ylethyl carbonate (known from WO2009/049851), 4-(but-2-yn-1-yloxy)-6-(3,5-dimethyl piperidin-1-yl)-5-fluoropyrimidine (known from WO2004/099160), 4-(but-2-yn-1-yloxy)-6-(3-chlorophenyl)pyrimidine (known from WO2003/076415), PF1364 (CAS Reg. No. 1204776-60-2), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazine carboxy late (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 8-chloro-N-[(2-chloro-5-methoxyphenyl)sulphonyl]-6-(trifluoromethyl)imidazo[1,2-a]pyridine-2-carboxamide (known from WO2010/129500), 4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2009/080250), N-[(2E)-1-[(6-chloropyridin-3-yl)methyl]pyridin-2(1H)-ylidene]-2,2,2-trifluoroacetamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido [1,2-a]pyrimidin-1-ium-2-olate (known from WO2009/099929), 4-(3-{2, 6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969, 3-[benzoyl(methyl)amino]-N-[2-bromo-4-[1,2,2,2-tetrafluoro-1-(trifluoromethyl)ethyl]-6-(trifluoromethyl)phenyl]-2-fluorobenzamide (known from WO 2010018714), butyl [2-(2,4-dichlorophenyl)-3-oxo-4-oxaspiro[4.5]dec-1-en-1-yl]carbonate (known from CN 102060818), 4-[5-(3,5-dichlorophenyl)-5-(trifluoromethyl)-4H-isoxazol-3-yl]-N—[(Z)-methoxyiminomethyl]-2-methylbenzamide (known from WO2007/026965), 3E)-3-[1-[(6-chloro-3-pyridyl)methyl]-2-pyridylidene]-1,1,1-trifluoropropan-2-one (known from WO2013/144213, N-(methylsulphonyl)-6-[2-(pyridin-3-yl)-1,3-thiazol-5-yl]pyridine-2-carboxamide (known from WO2012/000896), N-[3-(benzylcarbamoyl)-4-chlorophenyl]-1-methyl-3-(pentafluoroethyl)-4-(trifluoromethyl)-1H-pyrazole-5-carboxamide (known from WO2010/051926).

### Fungicides

The active ingredients specified herein by their common name are known and described, for example, in “Pesticide Manual” or on the Internet (for example: http://www.alanwood.net/pesticides).

All the fungicidal mixing components listed in classes (1) to (15) may optionally form salts with corresponding bases or acids if suitable functional groups are present. In addition, the fungicidal mixing components listed in classes (1) to (15) also include tautomeric forms if tautomerism is possible.

1) Ergosterol biosynthesis inhibitors, for example (1.01) aldimorph, (1.02) azaconazole, (1.03) bitertanol, (1.04) bromuconazole, (1.05) cyproconazole, (1.06) diclobutrazole, (1.07) difenoconazole, (1.08) diniconazole, (1.09) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamide, (1.17) fenpropidin, (1.18) fenpropimorph, (1.19) fluquinconazole, (1.20) flurprimidol, (1.21) flusilazole, (1.22) flutriafol, (1.23) furconazole, (1.24) furconazole-cis, (1.25) hexaconazole, (1.26) imazalil, (1.27) imazalil sulphate, (1.28) imibenconazole, (1.29) ipconazole, (1.30) metconazole, (1.31) myclobutanil, (1.32) naftifine, (1.33) nuarimol, (1.34) oxpoconazole, (1.35) paclobutrazole, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole, (1.48) terbinafin, (1.49) tetraconazole, (1.50) triadimefon, (1.51) triadimenol, (1.52) tridemorph, (1.53) triflumizole, (1.54) triforin, (1.55) triticonazole, (1.56) uniconazole, (1.57) uniconazole-p, (1.58) viniconazole, (1.59) voriconazole, (1.60) 1-(4-chlorophenyl)-2-(1H-1,2,4-triazol-1-yl)cycloheptanol, (1.61) methyl 1-(2,2-dimethyl-2,3-dihydro-1H-inden-1-yl)-1H-imidazole-5-carboxylate, (1.62) N′-{5-(difluoromethyl)-2-methyl-4-[3-(trimethylsilyl)propoxy]phenyl}-N-ethyl-N-methylimidoformamide, (1.63)N-ethyl-N-methyl-N′-{2-methyl-5-(trifluoromethyl)-4-[3-(trimethylsilyl)propoxy]phenyl}imidoformamide, (1.64) O-[1-(4-methoxyphenoxy)-3,3-dimethylbutan-2-yl]-1H-imidazole-1-carbothioate, (1.65) pyrisoxazole, (1.66) 2-{[3-(2-chlorophenyl)-2-(2,4-difluorophenyl)oxiran-2-yl]methyl}-2,4-dihydro-3H-1,2,4-triazole-3-thione, (1.67) 1-{[3-(2-chlorophenyl)-2-(2,4-difluorophenyl)oxiran-2-yl]methyl}-1H-1,2,4-triazol-5-ylthiocyanate, (1.68) 5-(allylsulphanyl)-1-{[3-(2-chlorophenyl)-2-(2,4-difluorophenyl)oxiran-2-yl]methyl}-1H-1,2,4-triazole, (1.69) 2-[1-(2,4-dichlorophenyl)-5-hydroxy-2,6,6-trimethylheptan-4-yl]-2,4-dihydro-3H-1,2,4-triazole-3-thione, (1.70) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.71) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.72) 1-{[rel(2R,3S)-3-(2-chlorophenyl)-2-(2,4-difluorophenyl)oxiran-2-yl]methyl}-1H-1,2,4-triazol-5-ylthiocyanate, (1.73) 1-{[rel(2R,3R)-3-(2-chlorophenyl)-2-(2,4-difluorophenyl)oxiran-2-yl]methyl}-1H-1,2,4-triazol-5-ylthiocyanate, {(1.74) 5-(allylsulphanyl)-1-[rel(2R,3S)-3-(2-chlorophenyl)-2-(2,4-difluorophenypoxiran-2-yl]methyl}-1H-1,2,4-triazole, (1.75) 5-(allylsulphanyl)-1-{[rel(2R,3R)-3-(2-chlorophenyl)-2-(2,4-difluorophenyl)oxiran-2-yl]methyl}-1H-1,2,4-triazole, (1.76) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.77) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.78) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.79) 2-[(2 S,4R,5R)-1-(2,4-dichlorophenyl)-5-hydroxy-2,6,6-trimethylheptan-4-yl]-2,4-dihydro-3H-1,2,4-triazole-3-thione, (1.80) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.81) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.82) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.83) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.84) 2-[4-(4-chlorophenoxy)-2-(trifluoromethyl)phenyl]-1-(1H-1,2,4-triazol-1-y 1)propan-2-ol, (1.85) 2-[4-(4-chlorophenoxy)-2-(trifluoromethyl)phenyl]-1-(1H-1,2,4-triazol-1-y 1)butan-2-ol, (1.86) 2-[4-(4-chlorophenoxy)-2-(trifluoromethyl)phenyl]-1-(1H-1,2,4-triazol-1-yl)pentan-2-ol, (1.87) 2-[2-chloro-4-(4-chlorophenoxy)phenyl]-1-(1H-1,2,4-triazol-1-y 1)butan-2-ol, (1.88) 2-[2-chloro-4-(2,4-dichlorophenoxy)phenyl]-1-(1H-1,2,4-triazol-1-yl)propan-2-ol, (1.89) (2R)-2-(1-chlorocyclopropyl)-4-[(1R)-2,2-dichlorocyclopropyl]-1-(1H-1,2,4-triazol-1-y 1)butan-2-ol, (1.90) (2R)-2-(1-chlorocyclopropyl)-4-[(1S)-2,2-dichlorocyclopropyl]-1-(1H-1,2,4-triazol-1-y 1)butan-2-ol, (1.91) (2S)-2-(1-chlorocyclopropyl)-4-[(1S)-2,2-dichlorocyclopropyl]-1-(1H-1,2,4-triazol-1-yl)butan-2-ol, (1.92) (2S)-2-(1-chlorocyclopropyl)-4-[(1R)-2,2-dichlorocyclopropyl]-1-(1H-1,2,4-triazol-1-y 1)butan-2-ol, (1.93) (1 S,2R,5R)-5-(4-chlorobenzyl)-2-(chloromethyl)-2-methyl-1-(1H-1,2,4-triazol-1-ylmethyl)cyclopentanol, (1.94) (1R,2S,5S)-5-(4-chlorobenzyl)-2-(chloromethyl)-2-methyl-1-(1H-1,2,4-triazol-1-ylmethyl)cyclopentanol, (1.95) 5-(4-chlorobenzyl)-2-(chloromethyl)-2-methyl-1-(1H-1,2,4-triazol-1-ylmethyl)cyclopentanol.

2) Inhibitors of complex I or II of the respiratory chain, for example (2.01) bixafen, (2.02) boscalid, (2.03) carboxin, (2.04) diflumetorim, (2.05) fenfuram, (2.06) fluopyram, (2.07) flutolanil, (2.08) fluxapyroxad, (2.09) furametpyr, (2.10) furmecyclox, (2.11) isopyrazam (mixture of syn-epimeric racemate 1RS,4SR,9RS and anti-epimeric racemate 1RS,4SR,9SR), (2.12) isopyrazam (anti-epimeric racemate 1RS,4SR,9SR), (2.13) isopyrazam (anti-epimeric enantiomer 1R,4S,9S), (2.14) isopyrazam (anti-epimeric enantiomer 1S,4R,9R), (2.15) isopyrazam (syn-epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad, (2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methy 1-1H-pyrazole-4-carboxamide, (2.27)N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazolin-4-amine, (2.29) benzovindiflupyr, (2.30)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.31)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3S)-1,1,3-trimethyl-2,3-dihydro-1H-inden-4-yl]-1H-pyrazole-4-carboxamide, (2.37) 3-(difluoromethyl)-1-methyl-N-[(3S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-1,1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil, (2.42) 2-chloro-N-(1,1,3-trimethyl-2,3-dihydro-1H-inden-4-yl)pyridine-3-carboxamide, (2.43) isofetamid, (2.44) 1-methyl-3-(trifluoromethyl)-N-[2′-(trifluoromethyl)biphenyl-2-yl]-1H-pyrazole-4-carboxamide, (2.45)N-(4′-chlorobiphenyl-2-yl)-3-(difluoromethyl)-1-methy 1-1H-pyrazole-4-carboxamide, (2.46)N-(2′,4′-dichlorobiphenyl-2-yl)-3-(difluoromethyl)-1-methy 1-1H-pyrazole-4-carboxamide, (2.47) 3-(difluoromethyl)-1-methyl-N-[4′-(trifluoromethyl)biphenyl-2-yl]-1H-pyrazole-4-carboxamide, (2.48)N-(2′,5′-difluorobiphenyl-2-yl)-1-methyl-3-(trifluoromethyl)-1H-pyrazole-4-carboxamide, (2.49) 3-(difluoromethyl)-1-methyl-N-[4′-(prop-1-yn-1-yl)biphenyl-2-yl]-1H-pyrazole-4-carboxamide, (2.50) 5-fluoro-1,3-dimethyl-N-[4′-(prop-1-yn-1-yl)biphenyl-2-yl]-1H-pyrazole-4-carboxamide, (2.51) 2-chloro-N-[4′-(prop-1-yn-1-yl)biphenyl-2-yl]nicotinamide, (2.52) 3-(difluoromethyl)-N-[4′-(3,3-dimethylbut-1-yn-1-yl)biphenyl-2-yl]-1-methyl-1H-pyrazole-4-carboxamide, (2.53)N-[4′-(3,3-dimethylbut-1-yn-1-yl)biphenyl-2-yl]-5-fluoro-1,3-dimethyl-1H-pyrazole-4-carboxamide, (2.54) 3-(difluoromethyl)-N-(4′-ethynylbiphenyl-2-yl)-1-methyl-1H-pyrazole-4-carboxamide, (2.55)N-(4′-ethynylbiphenyl-2-yl)-5-fluoro-1,3-dimethyl-1H-pyrazole-4-carboxamide, (2.56) 2-chloro-N-(4′-ethynylbiphenyl-2-yl)nicotinamide, (2.57) 2-chloro-N-[4′-(3,3-dimethylbut-1-yn-1-yl)biphenyl-2-yl]nicotinamide, (2.58) 4-(difluoromethyl)-2-methyl-N-[4′-(trifluoromethyl)biphenyl-2-yl]-1,3-thiazole-5-carboxamide, (2.59) 5-fluoro-N-[4′-(3-hydroxy-3-methylbut-1-yn-1-yl)biphenyl-2-yl]-1,3-dimethyl-1H-pyrazole-4-carboxamide, (2.60) 2-chloro-N-[4′-(3-hydroxy-3-methylbut-1-yn-1-yl)biphenyl-2-yl]nicotinamide, (2.61) 3-(difluoromethyl)-N-[4′-(3-methoxy-3-methylbut-1-yn-1-yl)biphenyl-2-yl]-1-methyl-1H-pyrazole-4-carboxamide, (2.62) 5-fluoro-N-[4′-(3-methoxy-3-methylbut-1-yn-1-yl)biphenyl-2-yl]-1,3-dimethyl-1H-pyrazole-4-carboxamide, (2.63) 2-chloro-N-[4′-(3-methoxy-3-methylbut-1-yn-1-yl)biphenyl-2-yl]nicotinamide, (2.64) 1,3-dimethyl-N-(1,1,3-trimethyl-2,3-dihydro-1H-inden-4-yl)-1H-pyrazole-4-carboxamide, (2.65) 1,3-dimethyl-N-[(3R)-1,1,3-trimethyl-2,3-dihydro-1H-inden-4-yl]-1H-pyrazole-4-carboxamide, (2.66) 1,3-dimethyl-N-[(3S)-1,1,3-trimethyl-2,3-dihydro-1H-inden-4-yl]-1H-pyrazole-4-carboxamide, (2.67) 3-(difluoromethyl)-N-methoxy-1-methyl-N-[1-(2,4,6-trichlorophenyl)propan-2-yl]-1H-pyrazole-4-carboxamide, (2.68) 3-(difluoromethyl)-N-(7-fluoro-1,1,3-trimethyl-2,3-dihydro-1H-inden-4-yl)-1-methy 1-1H-pyrazole-4-carboxamide, (2.69) 3-(difluoromethyl)-N-[(3R)-7-fluoro-1,1,3-trimethyl-2,3-dihydro-1H-inden-4-yl]-1-methyl-1H-pyrazole-4-carboxamide, (2.70) 3-(difluoromethyl)-N-[(3S)-7-fluoro-1,1,3-trimethyl-2,3-dihydro-1H-inden-4-yl]-1-methy 1-1H-pyrazole-4-carboxamide. 3) Inhibitors of the respiratory chain on complex III, for example (3.01) ametoctradin, (3.02) amisulbrom, (3.03) azoxystrobin, (3.04) cyazofamid, (3.05) coumethoxystrobin, (3.06) coumoxystrobin, (3.07) dimoxystrobin, (3.08) enoxastrobin, (3.09) famoxadon, (3.10) fenamidon, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylacetamide, (3.24) (2E)-2-(methoxyimino)-N-methyl-2-(2-{[({(1E)-1-[3-(trifluoromethyl)phenyl]ethyliden}amino)oxy]methyl}phenyl)acetamide, (3.25) (2E)-2-(methoxy imino)-N-methyl-2-{2-[(E)-({1-[3-(trifluoromethyl)phenyl]ethoxy}imino)methyl]phenyl}acetamide, (3.26) (2E)-2-{2-[({[(1E)-1-(3-{[(E)-1-fluoro-2-phenylvinyl]oxy}phenyl)ethyliden]amino}oxy)methyl]phenyl}-2-(methoxy imino)-N-methylacetamide, (3.27) fenaminostrobin, (3.28) 5-methoxy-2-methyl-4-(2-{[({(1E)-1-[3-(trifluoromethyl)phenyl]ethyliden}amino)oxy]methyl}phenyl)-2,4-dihydro-3H-1,2,4-triazol-3-one, (3.29) (2E)-2-{2-[({cyclopropyl[(4-methoxyphenyl)imino]methyl}sulphanyl)methyl]phenyl}-3-methoxyacrylic acid methyl ester, (3.30)N-(3-ethyl-3,5,5-trimethylcyclohexyl)-3-formamido-2-hydroxybenzamide, (3.31) 2-{2-[(2,5-dimethylphenoxy)methyl]phenyl}-2-methoxy-N-methylacetamide, (3.32) 2-{2-[(2,5-dimethylphenoxy)methyl]phenyl}-2-methoxy-N-methylacetamide, (3.33) (2E,3Z)-5-{[1-(4-chlorophenyl)-1H-pyrazol-3-yl]oxy}-2-(methoxy imino)-N,3-dimethylpent-3-enamide.

4) Mitosis and cell division inhibitors, for example (4.01) benomyl, (4.02) carbendazim, (4.03) chlorfenazole, (4.04) diethofencarb, (4.05) ethaboxam, (4.06) fluopicolide, (4.07) fuberidazole, (4.08) pencycuron, (4.09) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl) [1,2,4]triazolo [1,5-a]pyrimidine, (4.14) 3-chloro-5-(6-chloropyridin-3-yl)-6-methyl-4-(2,4,6-trifluorophenyl)pyridazine.

5) Compounds capable of having multisite action, for example (5.01) Bordeaux mixture, (5.02) captafol, (5.03) captan, (5.04) chlorothalonil, (5.05) copper hydroxide, (5.06) copper naphthenate, (5.07) copper oxide, (5.08) copper oxychloride, (5.09) copper(2+) sulphate, (5.10) dichlofluanid, (5.11) dithianon, (5.12) dodine, (5.13) dodine free base, (5.14) ferbam, (5.15) fluorofolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) metiram zinc, (5.27) oxine-copper, (5.28) propamidine, (5.29) propineb, (5.30) sulphur and sulphur preparations including calcium poly sulphide, (5.31) thiram, (5.32) tolylfluanid, (5.33) zineb, (5.34) ziram, (5.35) anilazine.

6) Compounds capable of inducing host defence, for example (6.01) acibenzolar-S-methyl, (6.02) isotianil, (6.03) probenazole, (6.04) tiadinil, (6.05) laminarin

7) Amino acid and/or protein biosynthesis inhibitors, for example (7.01) andoprim, (7.02) blasticidin-S, (7.03) cyprodinil, (7.04) kasugamycin, (7.05) kasugamycin hydrochloride hydrate, (7.06) mepanipyrim, (7.07) pyrimethanil, (7.08) 3-(5-fluoro-3,3,4,4-tetramethyl-3,4-dihydroisoquinolin-1-yl)quinoline, (7.09) oxytetracycline, (7.10) streptomycin.

8) ATP production inhibitors, for example (8.01) fentin acetate, (8.02) fentin chloride, (8.03) fentin hydroxide, (8.04) silthiofam.

9) Cell wall synthesis inhibitors, for example (9.01) benthiavalicarb, (9.02) dimethomorph, (9.03) flumorph, (9.04) iprovalicarb, (9.05) mandipropamid, (9.06) polyoxins, (9.07) polyoxorim, (9.08) validamycin A, (9.09) valifenalate, (9.10) polyoxin B, (9.11) (2E)-3-(4-tert-butylphenyl)-3-(2-chloropyridin-4-yl)-1-(morpholin-4-yl)prop-2-en-1-one, (9.12) (2Z)-3-(4-tert-butylphenyl)-3-(2-chloropyridin-4-yl)-1-(morpholin-4-yl)prop-2-en-1-one.

10) Lipid and membrane synthesis inhibitors, for example (10.01) biphenyl, (10.02) chloroneb, (10.03) dicloran, (10.04) edifenphos, (10.05) etridiazole, (10.06) iodocarb, (10.07) iprobenfos, (10.08) isoprothiolane, (10.09) propamocarb, (10.10) propamocarb hydrochloride, (10.11) prothiocarb, (10.12) pyrazophos, (10.13) quintozene, (10.14) tecnazene, (10.15) tolclofos-methyl.

11) Melanin biosynthesis inhibitors, for example (11.01) carpropamid, (11.02) diclocymet, (11.03) fenoxanil, (11.04) phthalide, (11.05) pyroquilon, (11.06) tricyclazole, (11.07) 2,2,2-trifluoroethyl {3-methyl-1-[(4-methylbenzoyl)amino]butan-2-yl}carbamate.

12) Nucleic acid synthesis inhibitors, for example (12.01) benalaxyl, (12.02) benalaxyl-M (kiralaxyl), (12.03) bupirimate, (12.04) clozylacon, (12.05) dimethirimol, (12.06) ethirimol, (12.07) furalaxyl, (12.08) hymexazole, (12.09) metalaxyl, (12.10) metalaxyl-M (mefenoxam), (12.11) ofurace, (12.12) oxadixyl, (12.13) oxolinic acid, (12.14) octhilinone.

13) Signal transduction inhibitors, for example (13.01) chlozolinate, (13.02) fenpiclonil, (13.03) fludioxonil, (13.04) iprodione, (13.05) procymidone, (13.06) quinoxyfen, (13.07) vinclozolin, (13.08) proquinazid.

14) Compounds capable of acting as uncouplers, for example (14.01) binapacryl, (14.02) dinocap, (14.03) ferimzone, (14.04) fluazinam, (14.05) meptyldinocap.

15) Further compounds, for example (15.001) benthiazole, (15.002) bethoxazin, (15.003) capsimycin, (15.004) carvone, (15.005) quinomethionate, (15.006) pyriofenone (chlazafenone), (15.007) cufraneb, (15.008) cyflufenamid, (15.009) cymoxanil, (15.010) cyprosulfamide, (15.011) dazomet, (15.012) debacarb, (15.013) dichlorophen, (15.014) diclomezin, (15.015) difenzoquat, (15.016) difenzoquat metilsulfate, (15.017) diphenylamine, (15.018) Ecomate, (15.019) fenpyrazamine, (15.020) flumetover, (15.021) fluoroimide, (15.022) flusulfamide, (15.023) flutianil, (15.024) fosetyl-aluminium, (15.025) fosetyl-calcium, (15.026) fosetyl-sodium, (15.027) hexachlorobenzene, (15.028) irumamycin, (15.029) methasulfocarb, (15.030) methyl isothiocyanate, (15.031) metrafenone, (15.032) mildiomycin, (15.033) natamycin, (15.034) nickel dimethyldithiocarbamate, (15.035) nitrothal-isopropyl, (15.036) oxamocarb, (15.037) oxyfenthiin, (15.038) pentachlorophenol and salts, (15.039) phenothrin, (15.040) phosphorous acid and salts thereof, (15.041) propamocarb-fosetylate, (15.042) propanosin-sodium, (15.043) pyrimorph, (15.044) pyrrolnitrin, (15.045) tebufloquin, (15.046) tecloftalam, (15.047) tolnifanid, (15.048) triazoxide, (15.049) trichlamid, (15.050) zarilamid, (15.051) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (15.052) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.053) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.054) oxathiapiproline, (15.055) 1-(4-methoxyphenoxy)-3,3-dimethylbutan-2-yl 1H-imidazole-1-carboxy late, (15.056) 2,3,5,6-tetrachloro-4-(methylsulphonyl)pyridine, (15.057) 2,3-dibutyl-6-chlorothieno[2,3-d]pyrimidin-4(3H)-one, (15.058) 2,6-dimethyl-1H,5H-[1,4]dithiino[2,3-c:5,6-c′]dipyrrole-1,3,5,7(2H,6H)-tetrone, (15.059) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (15.060) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, (15.061) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.062) 2-butoxy-6-iodo-3-propyl-4H-chromen-4-one, (15.063) 2-chloro-5-[2-chloro-1-(2,6-difluoro-4-methoxyphenyl)-4-methyl-1H-imidazol-5-yl]pyridine, (15.064) 2-phenylphenol and salts, (15.065) 3-(4,4,5-trifluoro-3,3-dimethyl-3,4-dihydroisoquinolin-1-yl)quinoline, (15.066) 3,4,5-trichloropyridine-2,6-dicarboxylic acid nitrile, (15.067) 3-chloro-5-(4-chlorophenyl)-4-(2,6-difluorophenyl)-6-methylpyridazine, (15.068) 4-(4-chlorophenyl)-5-(2,6-difluorophenyl)-3,6-dimethylpyridazine, (15.069) 5-amino-1,3,4-thiadiazole-2-thiol, (15.070) 5-chloro-N′-phenyl-N′-(prop-2-yn-1-yl)thiophene-2-sulphonohydrazide, (15.071) 5-fluoro-2-[(4-fluorobenzyl)oxy]pyrimidin-4-amine, (15.072) 5-fluoro-2-[(4-methylbenzyl)oxy]pyrimidin-4-amine, (15.073) 5-methyl-6-octyl[1,2,4]triazolo[1,5-a]pyrimidin-7-amine, (15.074) ethyl (2Z)-3-amino-2-cyano-3-phenylacrylate, (15.075) N′-(4-[3-(4-chlorobenzyl)-1,2,4-thiadiazol-5-yl]oxy-2,5-dimethylphenyl)-N-ethyl-N-methylimidoformamide, (15.076)N-(4-chlorobenzyl)-3-[3-methoxy-4-(prop-2-yn-1-yloxy)phenyl]propanamide, (15.077)N-[(4-chlorophenyl)(cyano)methyl]-3-[3-methoxy-4-(prop-2-yn-1-yloxy)phenyl]propanamide, (15.078)N-[(5-bromo-3-chloropyridin-2-yl)methyl]-2,4-dichloronicotinamide, (15.079)N-[1-(5-bromo-3-chloropyridin-2-yl)ethyl]-2,4-dichloronicotinamide, (15.080)N-[1-(5-bromo-3-chloropyridin-2-yl)ethyl]-2-fluoro-4-iodonicotinamide, (15.081)N—{(E)-[(cyclopropylmethoxy)imino][6-(difluoromethoxy)-2,3-difluorophenyl]methyl}-2-phenylacetamide, (15.082)N—{(Z)-[(cyclopropylmethoxy)imino][6-(difluoromethoxy)-2,3-difluorophenyl]methyl}-2-phenylacetamide, (15.083) N′-{4-[(3-tert-butyl-4-cyano-1,2-thiazol-5-yl)oxy]-2-chloro-5-methylphenyl}-N-ethyl-N-methylimidoformamide, (15.084)N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (15.085)N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (15.086)N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, (15.087) pentyl {6-[({[(1-methyl-1H-tetrazol-5-yl)(phenyl)methylene]amino}oxy)methyl]pyridin-2-yl}carbamate, (15.088) phenazine-1-carboxylic acid, (15.089) quinolin-8-ol, (15.090) quinolin-8-ol sulphate (2:1), (15.091) tert-butyl {6-[({[(1-methyl-1H-tetrazol-5-yl)(phenyl)methylene]amino}oxy)methyl]pyridin-2-yl}carbamate, (15.092) (5-bromo-2-methoxy-4-methylpyridin-3-yl)(2,3,4-trimethoxy-6-methylphenyl)methanone, (15.093)N-[2-(4-{[3-(4-chlorophenyl)prop-2-yn-1-yl]oxy}-3-methoxyphenyl)ethyl]-N2-(methylsulphonyl)valinamide, (15.094) 4-oxo-4-[(2-phenylethyl)amino]butanoic acid, (15.095) but-3-yn-1-yl {6-[({[(Z)-(1-methyl-1H-tetrazol-5-yl)(phenyl)methylene]amino}oxy)methyl]pyridin-2-yl}carbamate, (15.096) 4-amino-5-fluoropyrimidin-2-ol (tautomeric form: 4-amino-5-fluoropyrimidin-2(1H)-one), (15.097) propyl 3,4,5-trihydroxybenzoate, (15.098) [3-(4-chloro-2-fluorophenyl)-5-(2,4-difluorophenyl)-1,2-oxazol-4-yl](pyridin-3-yl)methanol, (15.099) (S)-[3-(4-chloro-2-fluorophenyl)-5-(2,4-difluorophenyl)-1,2-oxazol-4-yl](pyridin-3-yl)methanol, (15.100) (R)-[3-(4-chloro-2-fluorophenyl)-5-(2,4-difluorophenyl)-1,2-oxazol-4-yl](pyridin-3-yl)methanol, (15.101) 2-fluoro-6-(trifluoromethyl)-N-(1,1,3-trimethyl-2,3-dihydro-1H-inden-4-yl)benzamide, (15.102) 2-(6-benzylpyridin-2-yl)quinazoline, (15.103) 2-[6-(3-fluoro-4-methoxyphenyl)-5-methylpyridin-2-yl]quinazoline, (15.104) 3-(4,4-difluoro-3,3-dimethyl-3,4-dihydroisoquinolin-1-yl)quinoline, (15.105) abscisic acid, (15.106) N-[5-bromo-6-(2,3-dihydro-1H-inden-2-yloxy)-2-methylpyridin-3-yl]-N-ethyl-N-methylimidoformamide, (15.107) N′-{5-bromo-6-[1-(3,5-difluorophenyl)ethoxy]-2-methylpyridin-3-yl}-N-ethyl-N-methylimidoformamide, (15.108) N′-{5-bromo-6-[(1R)-1-(3,5-difluorophenyl)ethoxy]-2-methylpyridin-3-yl}-N-ethyl-N-methylimidoformamide, (15.109) N′-{5-bromo-6-[(1S)-1-(3,5-difluorophenyl)ethoxy]-2-methylpyridin-3-yl}-N-ethyl-N-methylimidoformamide, (15.110) N′-{5-bromo-6-[(cis-4-isopropylcyclohexyl)oxy]-2-methylpyridin-3-yl}-N-ethyl-N-methylimidoformamide, (15.111) N′-{5-bromo-6-[(trans-4-isopropylcyclohexyl)oxy]-2-methylpyridin-3-yl}-N-ethyl-N-methylimidoformamide, (15.112)N-cyclopropyl-3-(difluoromethyl)-5-fluoro-N-(2-isopropylbenzyl)-1-methyl-1H-pyrazole-4-carboxamide, (15.113)N-cyclopropyl-N-(2-cyclopropylbenzyl)-3-(difluoromethyl)-5-fluoro-1-methyl-1H-pyrazole-4-carboxamide, (15.114)N-(2-tert-butylbenzyl)-N-cyclopropyl-3-(difluoromethyl)-5-fluoro-1-methyl-1H-pyrazole-4-carboxamide, (15.115)N-(5-chloro-2-ethylbenzyl)-N-cyclopropyl-3-(difluoromethyl)-5-fluoro-1-methyl-1H-pyrazole-4-carboxamide, (15.116)N-(5-chloro-2-isopropylbenzyl)-N-cyclopropyl-3-(difluoromethyl)-5-fluoro-1-methyl-1H-pyrazole-4-carboxamide, (15.117)N-cyclopropyl-3-(difluoromethyl)-N-(2-ethyl-5-fluorobenzyl)-5-fluoro-1-methyl-1H-pyrazole-4-carboxamide, (15.118) N-cyclopropyl-3-(difluoromethyl)-5-fluoro-N-(5-fluoro-2-isopropylbenzyl)-1-methyl-1H-pyrazole-4-carboxamide, (15.119)N-cyclopropyl-N-(2-cyclopropyl-5-fluorobenzyl)-3-(difluoromethyl)-5-fluoro-1-methyl-1H-pyrazole-4-carboxamide, (15.120)N-(2-cyclopentyl-5-fluorobenzyl)-N-cyclopropyl-3-(difluoromethyl)-5-fluoro-1-methyl-1H-pyrazole-4-carboxamide, (15.121)N-cyclopropyl-3-(difluoromethyl)-5-fluoro-N-(2-fluoro-6-isopropylbenzyl)-1-methyl-1H-pyrazole-4-carboxamide, (15.122)N-cyclopropyl-3-(difluoromethyl)-N-(2-ethyl-5-methylbenzyl)-5-fluoro-1-methyl-1H-pyrazole-4-carboxamide, (15.123)N-cyclopropyl-3-(difluoromethyl)-5-fluoro-N-(2-isopropyl-5-methylbenzyl)-1-methyl-1H-pyrazole-4-carboxamide, (15.124)N-cyclopropyl-N-(2-cyclopropyl-5-methylbenzyl)-3-(difluoromethyl)-5-fluoro-1-methyl-1H-pyrazole-4-carboxamide, (15.125)N-(2-tert-butyl-5-methylbenzyl)-N-cyclopropyl-3-(difluoromethyl)-5-fluoro-1-methyl-1H-pyrazole-4-carboxamide, (15.126) N-[5-chloro-2-(trifluoromethyl)benzyl]-N-cyclopropyl-3-(difluoromethyl)-5-fluoro-1-methyl-1H-pyrazole-4-carboxamide, (15.127)N-cyclopropyl-3-(difluoromethyl)-5-fluoro-1-methyl-N-[5-methyl-2-(trifluoromethyl)benzyl]-1H-pyrazole-4-carboxamide, (15.128) N-[2-chloro-6-(trifluoromethyl)benzyl]-N-cyclopropyl-3-(difluoromethyl)-5-fluoro-1-methyl-1H-pyrazole-4-carboxamide, (15.129) N-[3-chloro-2-fluoro-6-(trifluoromethyl)benzyl]-N-cyclopropyl-3-(difluoromethyl)-5-fluoro-1-methyl-1H-pyrazole-4-carboxamide, (15.130)N-cyclopropyl-3-(difluoromethyl)-N-(2-ethyl-4,5-dimethylbenzyl)-5-fluoro-1-methyl-1H-pyrazole-4-carboxamide, (15.131)N-cyclopropyl-3-(difluoromethyl)-5-fluoro-N-(2-isopropylbenzyl)-1-methyl-1H-pyrazole-4-carbothioamide, (15.132) N′-(2,5-dimethyl-4-phenoxyphenyl)-N-ethyl-N-methylimidoformamide, (15.133) N′-{4-[(4,5-dichloro-1,3-thiazol-2-yl)oxy]-2,5-dimethylphenyl}-N-ethyl-N-methylimidoformamide, (15.134)N-(4-chloro-2,6-difluorophenyl)-4-(2-chloro-4-fluorophenyl)-1,3-dimethyl-1H-pyrazol-5-amine, (15.135) 9-fluoro-2,2-dimethyl-5-(quinolin-3-yl)-2,3-dihydro-1,4-benzoxazepine, (15.136) 2-{2-fluoro-6-[(8-fluoro-2-methylquinolin-3-yl)oxy]phenyl}propan-2-ol, (15.137) 2-{2-[(7,8-difluoro-2-methylquinolin-3-yl)oxy]-6-fluorophenyl}propan-2-ol, (15.138) 4-(2-chloro-4-fluorophenyl)-N-(2-fluorophenyl)-1,3-dimethyl-1H-pyrazol-5-amine, (15.139) 4-(2-chloro-4-fluorophenyl)-N-(2,6-difluorophenyl)-1,3-dimethyl-1H-pyrazol-5-amine, (15.140) 4-(2-chloro-4-fluorophenyl)-N-(2-chloro-6-fluorophenyl)-1,3-dimethyl-1H-pyrazol-5-amine, (15.141) 4-(2-bromo-4-fluorophenyl)-N-(2-chloro-6-fluorophenyl)-1,3-dimethyl-1H-pyrazol-5-amine, (15.142)N-(2-bromo-6-fluorophenyl)-4-(2-chloro-4-fluorophenyl)-1,3-dimethyl-1H-pyrazol-5-amine, (15.143) 4-(2-bromo-4-fluorophenyl)-N-(2-bromophenyl)-1,3-dimethyl-1H-pyrazol-5-amine, (15.144) 4-(2-bromo-4-fluorophenyl)-N-(2-bromo-6-fluorophenyl)-1,3-dimethyl-1H-pyrazol-5-amine, (15.145) 4-(2-bromo-4-fluorophenyl)-N-(2-chlorophenyl)-1,3-dimethyl-1H-pyrazol-5-amine, (15.146)N-(2-bromophenyl)-4-(2-chloro-4-fluorophenyl)-1,3-dimethyl-1H-pyrazol-5-amine, (15.147) 4-(2-chloro-4-fluorophenyl)-N-(2-chlorophenyl)-1,3-dimethyl-1H-pyrazol-5-amine, (15.148) 4-(2-bromo-4-fluorophenyl)-N-(2,6-difluorophenyl)-1,3-dimethyl-1H-pyrazol-5-amine, (15.149) 4-(2-bromo-4-fluorophenyl)-N-(2-fluorophenyl)-1,3-dimethyl-1H-pyrazol-5-amine, (15.150) N′-(4-{3-[(difluoromethyl)sulphanyl]phenoxy}-2,5-dimethylphenyl)-N-ethyl-N-methylimidoformamide, (15.151) N′-(2,5-dimethyl-4-{3-[(1,1,2,2-tetrafluoroethyl)sulphanyl]phenoxy}phenyl)-N-ethyl-N-methylimidoformamide, (15.152) N′-(2,5-dimethyl-4-{3-[(2,2,2-trifluoroethyl)sulphanyl]phenoxy}phenyl)-N-ethyl-N-methylimidoformamide, (15.153) N′-(2,5-dimethyl-4-{3-[(2,2,3,3-tetrafluoropropyl)sulphanyl]phenoxy}phenyl)-N-ethyl-N-methylimidoformamide, (15.154) N′-(2,5-dimethyl-4-{3-[(pentafluoroethyl)sulphanyl]phenoxy}phenyl)-N-ethyl-N-methylimidoformamide, (15.155) N′-(4-{[3-(difluoromethoxy)phenyl]sulphanyl}-2,5-dimethylphenyl)-N-ethyl-N-methylimidoformamide, (15.156) N′-(2,5-dimethyl-4-{[3-(1, 1,2,2-tetrafluoroethoxy)phenyl]sulphanyl}phenyl)-N-ethyl-N-methylimidoformamide, (15.157) N′-(2,5-dimethyl-4-{[3-(2, 2,2-trifluoroethoxy)phenyl]sulphanyl}phenyl)-N-ethyl-N-methylimidoformamide, (15.158) N′-(2,5-dimethyl-4-{[3-(2,2,3,3-tetrafluoropropoxy)phenyl]sulphanyl}phenyl)-N-ethyl-N-methylimidoformamide, (15.159) N′-(2,5-dimethyl-4-{[3-(pentafluoroethoxy)phenyl]sulphanyl}phenyl)-N-ethyl-N-methylimidoformamide, (15.160) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.161) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.162) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.163) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenylmethanesulphonate, (15.164) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulphonate, (15.165) 2-[3,5-bis(difluoromethyl)-1H-pyrazol-1-yl]-1-[4-(4-{(5S)-5-[2-(prop-2-yn-1-yloxy)phenyl]-4,5-dihydro-1,2-oxazol-3-yl}-1,3-thiazol-2-yl)piperidin-1-yl]ethanone, (15.166) 2-[3,5-bis(difluoromethyl)-1H-pyrazol-1-yl]-1-[4-(4-{(5R)-5-[2-(prop-2-yn-1-yloxy)phenyl]-4,5-dihydro-1,2-oxazol-3-yl}-1,3-thiazol-2-yl)piperidin-1-yl]ethanone, (15.167) 2-[3,5-bis(difluoromethyl)-1H-pyrazol-1-yl]-1-[4-(4-{(5S)-5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.168) 2-[3,5-bis(difluoromethyl)-1H-pyrazol-1-yl]-1-[4-(4-{(5R)-5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.169) 2-[3,5-bis(difluoromethyl)-1H-pyrazol-1-yl]-1-[4-(4-{(5S)-5-[2-chloro-6-(prop-2-yn-1-yloxy)phenyl]-4,5-dihydro-1,2-oxazol-3-yl}-1,3-thiazol-2-yl)piperidin-1-yl]ethanone, (15.170) 2-[3,5-bis(difluoromethyl)-1H-pyrazol-1-yl]-1-[4-(4-{(5R)-5-[2-chloro-6-(prop-2-yn-1-yloxy)phenyl]-4,5-dihydro-1,2-oxazol-3-yl}-1,3-thiazol-2-yl)piperidin-1-yl]ethanone, (15.171) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenylmethanesulphonate, (15.172) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1, 2-oxazol-5-yl}phenylmethanesulphonate, (15.173) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulphonate, (15.174) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulphonate.

### Biological Pesticides as Mixing Components

The compounds of the formula (I) can be combined with biological pesticides.

Biological pesticides include especially bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421) Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii, (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (new: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC₁ (Accession Number CBS 122089), Trichoderma harzianum, in particular T. harzianum rifai T39 (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safeners as Mixing Components

The compounds of the formula (I) can be combined with safeners, for example benoxacor, cloquintocet (-mexyl), cyometrinil, cyprosulphamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro [4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya bean, potato, sugar beet, sugar cane, tomatoes, bell peppers and chili peppers, cucumbers, melons, carrots, water melons, onions, lettuce, spinach, leeks, beans, Brassica oleracea (e.g. cabbage), peas and other vegetable species, cotton, tobacco, oilseed rape, and also fruit plants (with the fruits apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plants shall be understood to mean all developmental stages of the plants, for example seeds, cuttings and young (immature) plants up to mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested material (harvested plants or plant parts) and vegetative and generative propagation material, for example cuttings, tubers, rhizomes, slips and seeds.

The inventive treatment of the plants and parts of plants with the compounds of the formula (I) is effected directly or by allowing them to act on the surroundings, habitat or storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and parts thereof in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been grown by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better capability for storage and/or processability of the harvested products. Further and particularly emphasized examples of such properties are increased resistance of the plants against animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants against phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain herbicidally active ingredients, for example imidazolinones, sulphonylureas, glyphosates or phosphinothricin (for example the “PAT” gene). The genes which impart the desired properties (“traits”) in question may also be present in combinations with one another in the transgenic plants. Examples of transgenic plants include the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Crop Protection Types of Treatment

The plants and plant parts are treated with the compounds of the formula (I) directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, i.e. compounds of the formula (I) are applied to the foliage, where treatment frequency and the application rate should be adjusted according to the level of infestation with the pest in question.

In the case of systemically active ingredients, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvements. Nevertheless, the treatment of seed entails a series of problems which cannot always be solved in a satisfactory manner Thus, it is desirable to develop methods for protecting the seed and the germinating plant which dispense with, or at least reduce considerably, the additional application of pesticides during storage, after sowing or after emergence of the plants. It is additionally desirable to optimize the amount of active ingredient used so as to provide optimum protection for the seed and the germinating plant from attack by animal pests, but without damage to the plant itself by the active ingredient used. In particular, methods for the treatment of seed should also take account of the intrinsic insecticidal or nematicidal properties of pest-resistant or -tolerant transgenic plants in order to achieve optimal protection of the seed and the germinating plant with a minimum expenditure on pesticides.

The present invention therefore in particular also relates to a method for the protection of seed and germinating plants from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and a mixing component may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages encountered with a systemically acting compound of the formula (I) is the fact that, by treating the seed, not only the seed itself but also the plants resulting therefrom are, after emergence, protected against animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for protection of seed of any plant variety which is used in agriculture, in the greenhouse, in forests or in horticulture. More particularly, this includes seed of cereals (for example wheat, barley, rye, millet and oats), maize, cotton, soya beans, rice, potatoes, sunflowers, coffee, tobacco, canola, oilseed rape, beet (for example sugar beet and fodder beet), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate in this case from microorganisms such as Bacillus, Rhizobium, Pseudomonas, Serratia, Trichoderma, Clavibacter, Glomus or Gliocladium. The present invention is particularly suitable for the treatment of transgenic seed containing at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been pre-swollen in water up to a certain stage (pigeon breast stage) for example, which leads to improved germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active ingredients which can exhibit phytotoxic effects at certain application rates.

In general, the compounds of the formula (I) are applied to the seed in the form of a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are prepared in a known manner, by mixing compounds of the formula (I) with customary additives such as, for example, customary extenders and also solvents or diluents, dyes, wetting agents, dispersants, emulsifiers, antifoams, preservatives, secondary thickeners, adhesives, gibberellins and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of active agrochemical ingredients. Alkyl naphthalenesulphonates, such as diisopropyl or diisobutyl naphthalenesulphonates, can be used with preference.

Suitable dispersants and/or emulsifiers which may be present in the seed-dressing formulations usable in accordance with the invention are all nonionic, anionic and cationic dispersants customary for the formulation of active agrochemical ingredients. Nonionic or anionic dispersants or mixtures of nonionic or anionic dispersants can be used with preference. Suitable nonionic dispersants include in particular ethylene oxide/propylene oxide block polymers, alkylphenol polyglycol ethers and tristyrylphenol polyglycol ethers, and the phosphated or sulphated derivatives thereof. Suitable anionic dispersants are especially lignosulphonates, polyacrylic acid salts and arylsulphonate-formaldehyde condensates.

Antifoams which may be present in the seed-dressing formulations usable in accordance with the invention are all foam-inhibiting substances customary for the formulation of active agrochemical ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions.

Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz- and Schadlingsbekampfungsmittel”, vol. 2, Springer Verlag, 1970, pp. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seed of transgenic plants.

For the treatment of seed with the seed-dressing formulations usable in accordance with the invention, or use forms prepared therefrom, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. the field of veterinary medicine, the compounds of the formula (I) are active against animal parasites, in particular ectoparasites or endoparasites. The term “endoparasites” includes especially helminths and protozoa, such as coccidia. Ectoparasites are typically and preferably arthropods, especially insects and acarids.

In the field of veterinary medicine, the compounds of the formula (I) having favourable endotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals such as sheep, goats, horses, donkeys, camels, buffalo, rabbits, reindeer, fallow deer, and particularly cattle and pigs; poultry such as turkeys, ducks, geese, and particularly chickens; fish and crustaceans, for example in aquaculture, and also insects such as bees.

Domestic animals include, for example, mammals, such as hamsters, guinea pigs, rats, mice, chinchillas, ferrets, and particularly dogs, cats, caged birds, reptiles, amphibians and aquarium fish.

In a preferred embodiment, the compounds of the formula (I) are administered to mammals.

In another preferred embodiment, the compounds of the formula (I) are administered to birds, namely caged birds and particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of animal health, the term “control” or “controlling” means that the compounds of the formula (I) are effective in reducing the incidence of the particular parasite in an animal infected with such parasites to an innocuous degree. More specifically, “controlling” in the present context means that the compound of the formula (I) can kill the respective parasite, inhibit its growth, or inhibit its proliferation.

Arthropods include:

from the order Anoplurida, for example Haematopinus spp., Linognathus spp., Pediculus spp., Phtirus spp., Solenopotes spp.; from the order Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order Siphonapterida, for example Pulex spp., Ctenocephalides spp., Tunga spp., Xenopsylla spp., Ceratophyllus spp.;

from the order Heteropterida, for example Cimex spp., Triatoma spp., Rhodnius spp., Panstrongylus spp.; and also nuisance and hygiene pests from the order Blattarida.

Arthropods further include:

from the subclass Acari (Acarina) and the order Metastigmata, for example from the family Argasidae like Argas spp., Ornithodorus spp., Otobius spp., from the family Ixodidae like Ixodes spp., Amblyomma spp., Rhipicephalus (Boophilus) spp., Dermacentor spp., Haemophysalis spp., Hyalomma spp., Rhipicephalus spp. (the original genus of multi-host ticks); from the order Mesostigmata like Dermanyssus spp., Ornithonyssus spp., Pneumonyssus spp., Raillietia spp., Pneumonyssus spp., Sternostoma spp., Varroa spp., Acarapis spp.; from the order Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp, Laminosioptes spp.

Parasitic protozoa include:

Mastigophora (Flagellata), for example Trypanosomatidae, for example Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, for example Trichomonadidae, for example Giardia lamblia, G. canis;

Sarcomastigophora (Rhizopoda) such as Entamoebidae, for example Entamoeba histolytica, Hartmanellidae, for example Acanthamoeba sp., Harmanella sp.;

Apicomplexa (Sporozoa) such as Eimeridae, for example Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example Toxoplasma gondii, Hammondia heydornii, Neospora caninum, Besnoitia besnoitii; such as Sarcocystidae, for example Sarcocystis bovicanis, S. bovihominis, S. ovicanis, S. ovifelis, S. neurona, S. spec., S. suihominis, such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Pathogenic endoparasites which are helminths include Platyhelmintha (e.g. Monogenea, cestodes and trematodes), nematodes, Acanthocephala, and Pentastoma. These include:

Monogenea: for example: Gyrodactylus spp., Dactylogyrus spp., Polystoma spp.;

Cestodes: from the order of Pseudophyllidea, for example: Diphyllobothrium spp., Spirometra spp., Schistocephalus spp., Ligula spp., Bothridium spp., Diphlogonoporus spp.;

from the order Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.;

Trematodes: from the class of Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fascioloides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.;

Nematodes: Trichinellida, for example Trichuris spp., Capillaria spp., Paracapillaria spp., Eucoleus spp., Trichomosoides spp., Trichinella spp.,

from the order Tylenchida, for example: Micronema spp., Strongyloides spp.;

from the order Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cy stocaulus spp., Pneumostrongylus spp., Spicocaulus spp., Elaphostrongylus spp., Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.;

from the order Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;

Acanthocephala: from the order of Oligacanthorhynchida, for example: Macracanthorhynchus spp., Prosthenorchis spp.; from the order Polymorphida for example: Filicollis spp.; from the order Moniliformida for example: Moniliformis spp.;

from the order Echinorhynchida, for example Acanthocephalus spp., Echinorhynchus spp., Leptorhynchoides spp.;

Pentastoma: from the order Porocephalida, for example Linguatula spp.

In the veterinary field and in animal husbandry, the compounds of the formula (I) are administered by methods generally known in the art, such as via the enteral, parenteral, dermal or nasal route in the form of suitable preparations. Administration may be prophylactic or therapeutic.

Thus, one embodiment of the present invention refers to the use of a compound of the formula (I) as a medicament.

A further aspect refers to the use of a compound of the formula (I) as an antiendoparasitic agent, in particular a helminthicidal agent or antiprotozoic agent. Compounds of the formula (I) are suitable for use as an antiendoparasitic agent, especially as a helminthicidal agent or antiprotozoic agent, for example in animal breeding, in animal husbandry, in animal houses and in the hygiene sector.

A further aspect in turn relates to the use of a compound of the formula (I) as an antiectoparasitic agent, in particular an arthropodicide such as an insecticide or an acaricide. A further aspect relates to the use of a compound of the formula (I) as an antiectoparasitic agent, in particular an arthropodicide such as an insecticide or an acaricide, for example in animal husbandry, in animal breeding, in animal houses or in the hygiene sector.

### Vector Control

The compounds of the formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) to a host or after injection (for example malaria parasites by mosquitoes) into a host.

Examples of vectors and the diseases or pathogens they transmit are:
1) mosquitoes
   Anopheles: malaria, filariasis;
   Culex: Japanese encephalitis, filariasis, other viral diseases, transmission of worms;
   Aedes: yellow fever, dengue fever, filariasis, other viral diseases;
   Simuliidae: transmission of worms, in particular Onchocerca volvulus;
2) Lice: skin infections, epidemic typhus;
3) Fleas: plague, endemic typhus;
4) Flies: sleeping sickness (trypanosomiasis); cholera, other bacterial diseases;
5) Mites: acariosis, epidemic typhus, rickettsialpox, tularaemia, Saint Louis encephalitis, tick-borne encephalitis (TBE), Crimean-Congo haemorrhagic fever, borreliosis;
6) Ticks: borellioses such as Borrelia duttoni, tick-borne encephalitis, Q fever (Coxiella burnetii), babesioses (Babesia canis canis).

Examples of vectors in the context of the present invention are insects, such as aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dims (malaria) and Culex, lice, fleas, flies, mites and ticks, which can transmit pathogens to animals and/or humans

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forests, in gardens and in leisure facilities, and also in the protection of materials and stored products.

### Protection of Industrial Materials

The compounds of the formula (I) are suitable for protecting industrial materials against attack or destruction by insects, for example from the orders Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) are present as a ready-to-use pesticide, i.e. it can be applied to the material in question without further modifications. Suitable further insecticides or fungicides are in particular those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active ingredients, as antifouling agents.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids and mites encountered in enclosed spaces, for example dwellings, factory halls, offices, vehicle cabins. For controlling animal pests, the compounds of the formula (I) are used alone or in combination with other active ingredients and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or bait stations.

### Description of the Processes and Intermediates

The preparation and use examples which follow illustrate the invention without limiting it. The products were characterized by ¹H NMR spectroscopy and/or LC-MS (Liquid Chromatography Mass Spectrometry).

The logP values were determined in accordance with OECD Guideline 117 (EC Directive 92/69/EEC) by HPLC (high-performance liquid chromatography) using reversed-phase (RP) columns (C₁₈), by the following methods:

[a] The LC-MS determination in the acidic range is carried out at pH 2.7 with 0.1% aqueous formic acid and acetonitrile (contains 0.1% formic acid) as eluents; linear gradient from 10% acetonitrile to 95% acetonitrile.

[b] LC-MS determination in the neutral range is effected at pH 7.8 with 0.001 molar aqueous ammonium hydrogencarbonate solution and acetonitrile as eluents; linear gradient from 10% acetonitrile to 95% acetonitrile.

Calibration is effected using unbranched alkan-2-ones (having 3 to 16 carbon atoms) with known logP values (logP values determined on the basis of the retention times by linear interpolation between two successive alkanone s).

The NMR spectra were determined using a Bruker Avance 400 fitted with a flow probe head (volume 60 μl). In individual cases, the NMR spectra were measured with a Bruker Avance II 600.

The ¹H NMR data of selected examples are noted in the form of 1H-NMR peak lists. For each signal peak, first the δ value in ppm and then the signal intensity in round brackets are listed. The pairs of δ value-signal intensity numbers for different signal peaks are listed with separation from one another by semicolons.

The peak list for one example therefore has the form:

δ₁ (intensity 1); δ₂ (intensity 2); . . . ; δᵢ (intensity i); . . . ; δₙ (intensity n)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D6 and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in this case to identify the reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional NMR interpretation.

Further details of NMR peak lists can be found in the Research Disclosure Database Number 564025.

### General synthesis of 4,5-disubstituted 2-(hetaryl)-pyrazolo[3,4-b]pyridines of the formula (I)

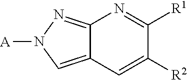

### EXAMPLE 1: 5-N-ACETYLAMINO-2-(3-PYRIDYL)-PYRAZOLO[3,4-B]PYRIDINE (A=3-PYRIDINYL; ═H, R²═NH—COCH₃)

### Step 1:

### Synthesis of 1-(3-pyridinyl)-1H-pyrazol-3-amine

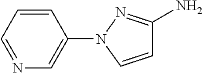 [SMILES: Nc1ccn(-c2cccnc2)n1]

To a solution of 260 g (1.61 mol) of 3-bromopyridine and 200 g (2.40 mol) of pyrazol-3-amine in 2.0 litres of N,N-dimethylformamide were added, with vigorous stirring, 994 g (7.2 mol) of potassium carbonate and 137 g (0.72 mol) of copper(I) iodide. Thereafter, stirring of the reaction mixture continued at 100° C. for 16 hours. For workup, the reaction mixture was poured into water and the precipitated solids were filtered off. The solids were then washed in diethyl ether and dried under reduced pressure. This gave 130 g (yield: 50.4% of theory) of 1-(3-pyridinyl)-1H-pyrazol-3-amine

### Step 2:

### 5-Nitro-2-(3-pyridyl)pyrazolo[3,4-b]pyridine (A=3-pyridinyl; R¹═H, R²═NO₂)

To a solution of 100 g (0.662 mol) of 1-(3-pyridinyl)-1H-pyrazol-3-amine and 72.0 g (0.662 mol) of sodium 2-nitropropanediolate hydrate (1:1:1) in 2.0 litres of N,N-dimethylformamide were added 550 ml of trimethylsilyl chloride, and the mixture was heated at reflux temperature for 12 hours. After the reaction had ended, the mixture was cooled to room temperature, and the solids were filtered off and washed with 300 ml of water. After drying, 76 g (yield: 50.2% of theory) of 5-nitro-2-(3-pyridyl)-pyrazolo[3,4-b]pyridine were obtained as a pale yellow solid.

### Step 3:

### 5-Amino-2-(3-pyridyl)-pyrazolo[3,4-b]pyridine (A=3-pyridinyl; R¹═H, R²═NH₂)

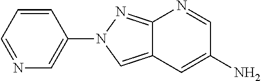 [SMILES: Nc1cnc2nn(-c3cccnc3)cc2c1]

To a stirred solution of 70 g (0.290 mol) of 5-nitro-2-(3-pyridyl)-pyrazolo[3,4-b]pyridine in 1.4 litres of acetic acid were added, at 0° C., 188.06 g (2.9 mol) of zinc. The mixture was stirred until the temperature rose to room temperature. After the reaction had ended, the reaction mixture was poured onto ice. Thereafter, ethyl acetate was added, and the organic phase formed was removed and dried over sodium sulphate. After the organic phase had been concentrated under reduced pressure, 51 g of crude product were obtained, which, after purification by column chromatography, gave 500 mg (yield: 5% of theory) of 5-amino-2-(3-pyridyl)-pyrazolo[3,4-b]pyridine in the form of a yellow solid.

### Step 4

### 5-N-Acetylamino-2-(3-pyridyl)-pyrazolo[3,4-b]pyridine (A=3-pyridinyl; R¹═H, R²═NH—COCH₃)

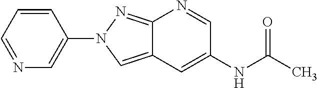 [SMILES: CC(=O)Nc1cnc2nn(-c3cccnc3)cc2c1]

To an initial charge of 0.20 g (0.947 mmol) of 5-amino-2-(3-pyridyppyrazolo[3,4-b]pyridine in about 80 ml of pyridine at 0° C. were added dropwise 1.20 g (1.174 mmol) of acetic anhydride, and then the mixture was stirred at room temperature for about 18 hours. Subsequently, the reaction mixture was concentrated under reduced pressure and the remaining residue was chromatographed by means of column chromatography (40 g RP column; gradient: acetonitrile/water; 5% acetonitrile (5 min) 45% acetonitrile (17 min), then in 17 min to 95% acetonitrile; flow rate: 35 ml/min). This gave 99 mg (99.4% purity, yield: 41% of theory) of 5-N-acetylamino-2-(3-pyridyppyrazolo[3,4-b]pyridine.

¹H-NMR (400.0 MHz, d₆-DMSO): δ=2.12 (s, 3H, CH₃); 7.65-7.68; 8.46-8.49; 8.62-8.68 (4 m, 5H, hetaryl); 9.17 (s, 1H, hetaryl); 9.31 (d, 1H; hetaryl); 10.30 (s, 1H, NH) ppm.

LC-MS (m/z): 254.1 (M+1); C₁₃H₁₁N₅O (253.25 g/mol)

### EXAMPLE 2: 5-[2-FLUORO-4-METHYL-5-(2,2,2-TRIFLUOROETHYLSULPHANYL)PHENYL]-2-(3-PYRIDYL)PYRAZOLO[3,4-B]PYRIDINE

### Step 1:

### Synthesis of 2-azido-5-bromopyridine-3-carbaldehyde

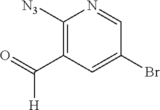 [SMILES: [N-]=[N+]=Nc1ncc(Br)cc1C=O]

A 250 ml round-bottom flask was charged with 4.85 g (23.77 mmol) of 5-bromo-2-fluoropyridine-3-carbaldehyde, 1.85 g (28.46 mmol) of sodium azide, 880 mg (2.38 mmol) of tetra-n-butylammonium iodide and 30 ml of dimethyl sulphoxide. Thereafter, the resulting reaction mixture was stirred at room temperature for 1 hour. This procedure was repeated four times. The resultant solutions were combined and diluted with 600 ml of water. The precipitated solids were filtered off and washed three times with 100 ml of water. Subsequently, the solids were dried under reduced pressure. This gave 15.6 g (yield: 72% of theory) of 2-azido-5-bromopyridine-3-carbaldehyde as a grey solid.

### Step 2:

### Synthesis of N-[(2-azido-5-bromopyridin-3-yl)methylidene]pyridin-3-amine

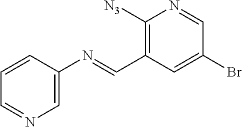 [SMILES: [N-]=[N+]=Nc1ncc(Br)cc1/C=N/c1cccnc1]

In a 250 ml round-bottom flask, 3.9 g (17.18 mmol) of 2-azido-5-bromopyridine-3-carbaldehyde (Example 2, Step 1), 2.43 g (25.82 mmol) of pyridin-3-amine and 78 ml of ethanol were stirred. Thereafter, the reaction solution was heated at reflux temperature on an oil bath for 6 hours. This procedure was repeated four times. The precipitated solids were filtered off and washed three times with 100 ml of petroleum ether. Thereafter, the solids were dried under reduced pressure. This gave 16.1 g (yield: 77% of theory) of N-[(2-azido-5-bromopyridin-3-yl)methylidene]pyridin-3-amine in solid form.

### Step 3:

### Synthesis of 5-bromo-2-(3-pyridyl)pyrazolo[3,4-b]pyridine

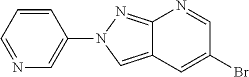 [SMILES: Brc1cnc2nn(-c3cccnc3)cc2c1]

In a 250 ml round-bottom flask, 4 g (13.20 mmol) of N-[(2-azido-5-bromopyridin-3-yl)methylidene]pyridin-3-amine were stirred in 80 ml of toluene. Thereafter, the reaction mixture was heated at reflux temperature on an oil bath for 6 hours. This procedure was repeated four times. The precipitated solids were filtered off and washed three times with 100 ml of petroleum ether. Thereafter, the solids were dried under reduced pressure. This gave 12 g (yield: 82% of theory) of 5-bromo-2-(3-pyridyppyrazolo[3,4-b]pyridine in the form of a brown solid.

¹H-NMR (400.0 MHz, CDCl₃): δ=9.25 (d, J=2.4 Hz, 1H), 8.78 (d, J=2.4 Hz, 1H), 8.74 (dd, J=1.2 Hz, 4.8 Hz, 1H), 8.50 (s, 1H), 8.42 (m, 1H), 8.30 (d, J=2.4 Hz, 1H), 7.57 (dd, J=4.8 Hz, 8.4 Hz, 1H) ppm.

LC-MS (ES, m/z): 274.9, 276.9 [M+H]

### Step 4:

### 5-[2-Fluoro-4-methyl-5-(2,2,2-trifluoroethylsulphanyl)phenyl]-2-(3-pyridyl)pyrazolo[3,4-b]pyridine

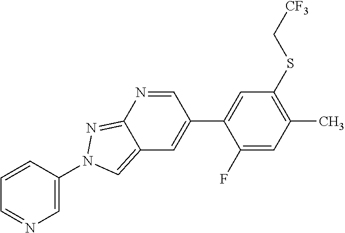 [SMILES: Cc1cc(F)c(-c2cnc3nn(-c4cccnc4)cc3c2)cc1SCC(F)(F)F]

To a mixture of 100 mg (363 μmol) of 5-bromo-2-(3-pyridyppyrazolo[3,4-b]pyridine (Example 2, Step 3) and 105 mg (392 μmol) of [2-fluoro-4-methyl-5-(2,2,2-trifluoroethylsulphanyl) phenyl]boronic acid was added a solution of 77 mg (0.73 mmol) of sodium carbonate in 375 μl of water and 1.5 ml of 1,4-dioxane. The reaction mixture was repeatedly purged with a stream of argon, 15 mg (19 μmol) of [1,1′-bis(diphenylphosphino)ferrocene]dichloropalladium(II) were added and the vessel was closed. The mixture was heated to 90° C. in a CEM Discover microwave reactor for 40 min and, after cooling to room temperature, filtered through a depth filter which was rinsed with ethyl acetate. After the solvent has been removed under reduced pressure, the residue was separated chromatographically by MPLC on silica gel (gradient: ethyl acetate/cyclohexane 0:100→100:0).

This gave a fraction of 42 mg (100% purity, yield: 27%) and a further fraction of 14 mg (94% purity, 9% yield) of 5-[2-fluoro-4-methyl-5-(2,2,2-trifluoroethylsulphanyl)phenyl]-2-(3-pyridyppyrazolo[3,4-b]pyridine.

¹H-NMR (400.0 MHz, d₆-DMSO): δ=9.402 (3.4); 9.395 (3.4); 9.369 (9.7); 8.909 (2.2); 8.904 (4.1); 8.899 (2.3); 8.726 (2.3); 8.723 (2.6); 8.714 (2.5); 8.711 (2.6); 8.582 (1.3); 8.579 (1.6); 8.576 (1.5); 8.572 (1.4); 8.561 (1.5); 8.558 (1.5); 8.555 (1.7); 8.551 (1.4); 8.499 (2.7); 8.496 (3.2); 8.494 (3.1); 8.491 (2.7); 7.859 (3.1); 7.840 (3.1); 7.719 (1.9); 7.707 (1.8); 7.698 (1.8); 7.686 (1.7); 7.390 (2.7); 7.361 (2.6); 4.118 (1.3); 4.092 (4.1); 4.066 (4.2); 4.040 (1.5); 3.330 (28.9); 2.677 (0.4); 2.673 (0.5); 2.668 (0.4); 2.526 (1.5); 2.513 (29.3); 2.508 (59.9); 2.504 (79.1); 2.499 (58.1); 2.495 (28.8); 2.458 (16.0); 2.335 (0.4); 2.331 (0.5); 2.326 (0.4); 1.990 (0.5); 0.146 (0.6); 0.008 (4.6); 0.000 (127.8); −0.009 (5.3); −0.150 (0.6) ppm.

### EXAMPLE 3: 5-[2-FLUORO-4-METHYL-5-(2,2,2-TRIFLUOROETHYLSULPHINYL)PHENYL]-2-(3-PYRIDYL)PYRAZOLO[3,4-B]PYRIDINE

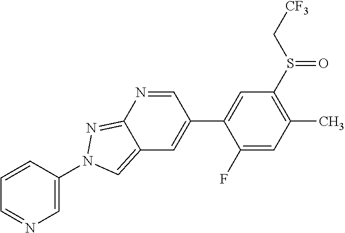 [SMILES: Cc1cc(F)c(-c2cnc3nn(-c4cccnc4)cc3c2)cc1S(=O)CC(F)(F)F]

18 mg (73 μmol) of 70% meta-chloroperbenzoic acid were added at 0° C. to a solution of 30 mg (2 μmol) of 5-[2-fluoro-4-methyl-5-(2,2,2-trifluoroethylsulphanyl)phenyl]-2-(3-pyridyl)pyrazolo[3,4-b]pyridine (Example 2) in 1.5 ml of methylene chloride. The reaction mixture was stirred at room temperature for 2 hours, and then saturated sodium carbonate solution was added. After 15 minutes, the phases were separated, the aqueous phase was extracted with methylene chloride and the combined organic phases were dried over sodium sulphate. The solvent was removed under reduced pressure and the residue was separated by chromatography by MPLC on silica gel (gradient: ethyl acetate/cyclohexane 0:100->100:0). This gave 30 mg (100% purity, yield: 95%) of 5-[2-fluoro-4-methyl-5-(2,2,2-trifluoroethylsulphinyl)phenyl]-2-(3-pyridyl)pyrazolo [3,4-b]pyridine.

¹H-NMR (400.0 MHz, d₆-DMSO): δ=9.408 (3.5); 9.402 (3.5); 9.389 (9.4); 8.970 (2.2); 8.965 (4.0); 8.960 (2.3); 8.732 (2.4); 8.729 (2.6); 8.720 (2.5); 8.717 (2.6); 8.589 (4.5); 8.585 (4.7); 8.583 (4.2); 8.568 (1.5); 8.565 (1.6); 8.562 (1.7); 8.558 (1.4); 8.318 (0.4); 8.117 (3.2); 8.097 (3.2); 7.724 (1.8); 7.713 (1.8); 7.704 (1.8); 7.693 (1.7); 7.504 (2.5); 7.476 (2.5); 4.335 (0.7); 4.325 (0.5); 4.307 (0.8); 4.298 (1.4); 4.280 (0.4); 4.271 (1.7); 4.243 (1.7); 4.215 (1.5); 4.206 (0.8); 4.188 (0.6); 4.179 (0.7); 4.038 (0.4); 4.020 (0.4); 3.330 (87.7); 2.676 (0.7); 2.672 (0.9); 2.667 (0.7); 2.525 (2.7); 2.512 (54.7); 2.507 (109.0); 2.503 (142.0); 2.498 (103.8); 2.494 (51.5); 2.476 (16.0); 2.334 (0.7); 2.330 (0.9); 2.325 (0.7); 1.989 (1.6); 1.193 (0.4); 1.175 (0.9); 1.158 (0.4); 0.146 (0.7); 0.008 (5.8); 0.000 (150.5); −0.009 (5.9); −0.150 (0.7) ppm.

### EXAMPLE 4: 5-[4-METHYL-3-(2,2,2-TRIFLUOROETHYLSULPHANYL)PHENYL]-2-(3-PYRIDYL)PYRAZOLO [3,4-B]PYRIDINE

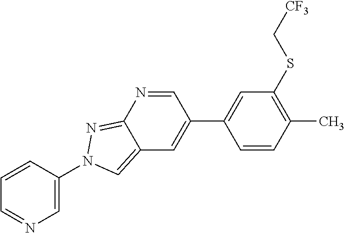 [SMILES: Cc1ccc(-c2cnc3nn(-c4cccnc4)cc3c2)cc1SCC(F)(F)F]

The preparation of 5-[4-methyl-3-(2,2,2-trifluoroethylsulphanyl)phenyl]-2-(3-pyridyl)pyrazolo[3,4-b]pyridine was effected in analogy to the synthesis of 5-[2-fluoro-4-methyl-5-(2,2,2-trifluoroethylsulphanyl)phenyl]-2-(3-pyridyppyrazolo[3,4-b]pyridine (cf. Example 2). This was done using 100 mg (365 μmol) of 5-bromo-2-(3-pyridyppyrazolo[3,4-b]pyridine (Example 2, Step 3) and 98 mg (0.39 mmol) of [4-methyl-3-(2,2,2-trifluoroethylsulphanyl)phenyl]boronic acid. This gave 89 mg (95% purity, yield: 58%) of 5-[4-methyl-3-(2,2,2-trifluoroethylsulphanyl)phenyl]-2-(3-pyridyppyrazolo[3,4-b]pyridine.

¹H-NMR (400.0 MHz, d₆-DMSO): δ=9.409 (3.4); 9.403 (3.6); 9.342 (9.9); 9.330 (0.5); 9.106 (4.9); 9.100 (5.1); 8.719 (2.5); 8.715 (2.7); 8.707 (2.7); 8.703 (2.8); 8.586 (1.4); 8.583 (1.7); 8.579 (1.7); 8.576 (1.7); 8.570 (5.2); 8.565 (6.2); 8.559 (2.2); 8.555 (1.7); 7.925 (3.5); 7.921 (3.7); 7.714 (2.0); 7.702 (1.9); 7.693 (1.9); 7.682 (1.9); 7.680 (1.8); 7.657 (2.0); 7.652 (2.0); 7.637 (2.2); 7.632 (2.2); 7.416 (3.1); 7.396 (2.6); 4.228 (1.3); 4.202 (4.0); 4.176 (4.2); 4.150 (1.4); 3.333 (55.0); 2.678 (0.4); 2.673 (0.5); 2.669 (0.4); 2.526 (1.5); 2.521 (2.2); 2.513 (30.9); 2.508 (63.6); 2.504 (83.6); 2.499 (60.6); 2.495 (29.6); 2.420 (16.0); 2.369 (0.7); 2.335 (0.4); 2.331 (0.6); 2.326 (0.5); 1.990 (0.3); 0.008 (1.6); 0.000 (51.4); −0.009 (1.9) ppm.

### EXAMPLE 5: 5-[4-METHYL-3-(2,2,2-TRIFLUOROETHYLSULPHINYL)PHENYL]-2-(3-PYRIDYL)PYRAZOLO [3,4-B]PYRIDINE

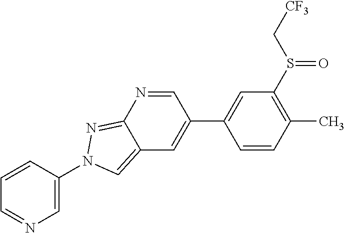 [SMILES: Cc1ccc(-c2cnc3nn(-c4cccnc4)cc3c2)cc1S(=O)CC(F)(F)F]

The preparation of 5-[4-methyl-3-(2,2,2-trifluoroethylsulphinyl)phenyl]-2-(3-pyridyppyrazolo[3,4-b]pyridine was effected in analogy to the synthesis of 5-[2-fluoro-4-methyl-5-(2,2,2-trifluoroethylsulphinyl)phenyl]-2-(3-pyridyppyrazolo[3,4-b]pyridine (Example 3). This was done using 60 mg (0.14 mmol) of 5-[4-methyl-3-(2,2,2-trifluoroethylsulphanyl)phenyl]-2-(3-pyridyppyrazolo[3,4-b]pyridine and 36 mg (0.14 mmol) of meta-chloroperbenzoic acid (70% purity). The crude product was admixed with dichloromethane and an insoluble precipitate was removed and then dried. In this way, 13 mg (89% purity, 19% yield) of 5-[4-methyl-3-(2,2,2-trifluoroethylsulphinyl)phenyl]-2-(3-pyridyppyrazolo[3,4-b]pyridine were obtained. The filtrate was concentrated under reduced pressure and the residue was separated by chromatography by MPLC on silica gel (gradient: ethyl acetate/cyclohexane 0:100→100:0). In this way, two further fractions, 22 mg (99% purity, 37% yield) and 5 mg (91% purity, yield: 8%), of 5-[4-methyl-3-(2,2,2-trifluoroethylsulphinyl)phenyl]-2-(3-pyridyppyrazolo[3,4-b]pyridine were obtained.

¹H-NMR (400.0 MHz, d₆-DMSO): δ=9.413(3.6); 9.406(3.7); 9.361(9.9); 9.144(5.0); 9.138(5.1); 8.725(2.6); 8.722(2.8); 8.713(2.7); 8.710(2.8); 8.646(5.1); 8.640(5.0); 8.591(1.5); 8.587(1.7); 8.584(1.6); 8.580(1.5); 8.570(1.6); 8.566(1.7); 8.563(1.8); 8.560(1.5); 8.241(4.0); 8.236(4.2); 7.983(2.0); 7.978(1.9); 7.963(2.2); 7.958(2.2); 7.720(2.0); 7.708(1.9); 7.699(2.0); 7.687(1.9); 7.527(3.0); 7.507(2.7); 5.759(0.4); 4.303(0.6); 4.294(0.5); 4.276(0.8); 4.266(1.6); 4.249(0.7); 4.239(1.6); 4.222(1.5); 4.212(0.8); 4.195(1.7); 4.186(0.8); 4.167(0.6); 4.158(0.7); 3.333(27.3); 2.677(0.3); 2.673(0.4); 2.668(0.3); 2.526(1.0); 2.513(24.1); 2.509(49.3); 2.504(64.7); 2.499(47.5); 2.495(23.7); 2.460(16.0); 2.331(0.4); 0.146(0.5); 0.008(4.0); 0.000(98.7); −0.009(4.5); −0.015(0.7); −0.018(0.7); −0.021(0.6); −0.150(0.4) ppm.

### EXAMPLE 6: 3-METHYLSULPHANYL-N-[2-(3-PYRIDYL)PYRAZOLO[3,4-B]PYRIDIN-5-YL]PROPANAMIDE

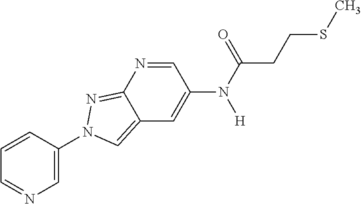 [SMILES: CSCCC(=O)Nc1cnc2nn(-c3cccnc3)cc2c1]

To a suspension of 100 mg (0.47 mmol) of 2-(3-pyridyppyrazolo[3,4-b]pyridin-5-amine (Example 1, Step 3) in 2.4 ml of acetonitrile and 0.15 ml of pyridine was added, at 0° C., a solution of 0.06 ml (0.5 mmol) of 3-methylthiopropionyl chloride in 2.4 ml of acetonitrile. The reaction mixture was stirred at room temperature overnight. Subsequently, 1 ml of pyridine was added to the suspension, which was stirred at room temperature for 3 h, and then a further 27.5 μl (238 μmol) of 3-methylthiopropionyl chloride were added at 0° C. The reaction mixture was stirred at room temperature overnight, and then another 13.8 μl (119 μmol) of 3-methylthiopropionyl chloride were added at 0° C. After stirring at room temperature for a further 3 h, the reaction mixture was diluted with water and extracted repeatedly with ethyl acetate. The combined organic phases were filtered. The precipitate removed was dried and, in this way, 29 mg (97% purity, 19% yield) of 3-methylsulphanyl-N-[2-(3-pyridyppyrazolo[3,4-b]pyridin-5-yl]propanamide were obtained. The filtrate was concentrated under reduced pressure. The residue was taken up in ethyl acetate and washed repeatedly with a saturated aqueous sodium hydrogencarbonate solution. The organic phase was then dried with sodium sulphate and the solvents were removed under reduced pressure. This gave 49 mg (98% purity, yield: 32%) of 3-methylsulphanyl-N-[2-(3-pyridyl)pyrazolo [3,4-b]pyridin-5-yl]propanamide

¹H-NMR (400.0 MHz, d₆-DMSO): δ=10.355(2.1); 9.330(2.1); 9.323(2.1); 9.192(5.3); 8.685(1.5); 8.682(1.5); 8.673(1.7); 8.670(2.0); 8.660(5.0); 8.657(4.8); 8.651(1.1); 8.503(0.8); 8.500(0.9); 8.497(0.9); 8.493(0.8); 8.482(0.9); 8.479(1.0); 8.476(1.0); 8.472(0.8); 7.686(1.1); 7.674(1.1); 7.665(1.1); 7.653(1.1); 3.506(0.3); 3.378(3.0); 2.813(1.0); 2.796(2.9); 2.779(2.2); 2.712(2.1); 2.695(2.7); 2.677(1.3); 2.508(50.0); 2.503(63.8); 2.499(46.2); 2.330(0.4); 2.116(16.0); 1.990(0.4); 1.298(0.4); 1.259(0.6); 1.232(0.7); 0.146(0.4); 0.008(3.3); 0.000(81.7); −0.008(3.3); −0.150(0.4) ppm.

### EXAMPLE 7: 3,3-DIFLUORO-N-[2-(3-PYRIDYL)PYRAZOLO[3,4-B]PYRIDIN-5-YL]BUTANAMIDE

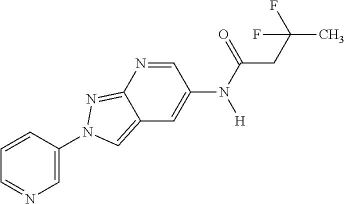 [SMILES: CC(F)(F)CC(=O)Nc1cnc2nn(-c3cccnc3)cc2c1]

To a suspension of 100 mg (0.47 mmol) of 2-(3-pyridyppyrazolo[3,4-b]pyridin-5-amine (Example 1, Step 3) in 2.4 ml of acetonitrile and 0.15 ml of pyridine was added, at 0° C., a solution of 67 mg (0.47 mmol) of 3,3-difluorobutanoyl chloride in 2.4 ml of acetonitrile. The reaction mixture was stirred at room temperature overnight. Subsequently, at 0° C., another solution of 67 mg (0.47 mmol) of 3,3-difluorobutanoyl chloride dissolved in 1 ml of acetonitrile was added. The reaction mixture was stirred at room temperature overnight, diluted with water and extracted repeatedly with ethyl acetate. The combined organic phases were dried with sodium sulphate and the solvents were removed under reduced pressure. The residue was separated chromatographically by MPLC on silica gel (gradient:methanol/dichloromethane 0:100→10:90). Subsequently, the crude product obtained was taken up in ethyl acetate and washed repeatedly with a saturated aqueous sodium hydrogencarbonate solution. The organic phase was dried with sodium sulphate and the solvents were removed under reduced pressure. This gave 57 mg (94% purity, 36% yield) of 3,3-difluoro-N-[2-(3-pyridyppyrazolo[3,4-b]pyridin-5-yl]butanamide. Reextraction of the combined aqueous sodium hydrogencarbonate-containing wash solutions with ethyl acetate gave a second amount of 33 mg (93% purity, yield: 21%) of 3,3-difluoro-N-[2-(3-pyridyppyrazolo[3,4-b]pyridin-5-yl]butanamide.

¹H-NMR (400.0 MHz, d₆-DMSO): δ=10.530(4.6); 9.332(4.7); 9.326(4.7); 9.202(12.5); 9.192(0.5); 8.690(3.3); 8.686(3.7); 8.678(3.5); 8.675(3.7); 8.659(2.4); 8.651(16.0); 8.507(1.9); 8.503(2.2); 8.500(2.2); 8.497(2.0); 8.486(2.0); 8.482(2.3); 8.479(2.4); 8.476(2.0); 8.314(0.8); 7.686(2.6); 7.674(2.5); 7.665(2.4); 7.655(2.3); 7.653(2.3); 3.320(135.0); 3.171(3.1); 3.133(6.5); 3.096(3.3); 2.676(1.0); 2.671(1.3); 2.667(1.0); 2.524(4.2); 2.511(69.5); 2.507(137.1); 2.502(180.3); 2.498(136.2); 2.493(70.4); 2.333(0.8); 2.329(1.1); 2.325(0.8); 1.861(5.5); 1.813(11.6); 1.765(5.9); 1.234(0.8); 0.146(0.6); 0.008(5.9); 0.000(133.4); −0.009(5.6); −0.150(0.5) ppm.

### EXAMPLE 8: 5-(1-METHYLPYRAZOL-4-YL)-2-(3-PYRIDYL)PYRAZOLO[3,4-B]PYRIDINE

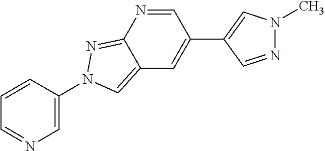 [SMILES: Cn1cc(-c2cnc3nn(-c4cccnc4)cc3c2)cn1]

In accordance with the reaction method from Ch. O. Ndubaku et al., J. Med. Chem., 2013, 56, 4597-4610, 1.2 ml of water and 0.6 ml of acetonitrile were added to a mixture of 100 mg (365 μmol) of 5-bromo-2-(3-pyridyppyrazolo[3,4-b]pyridine (Example 2, Step 3) and 107 mg (1.09 mmol) of potassium acetate. The reaction mixture was repeatedly purged with a stream of argon, and then 114 mg (547 μmol) of 1-methyl-4-(4,4,5,5-tetramethyl-1,3,2-dioxaborolan-2-yl)-1H-pyrazole as a solution in 0.6 ml of acetonitrile followed by 42 mg (36 μmol) of tetrakis(triphenylphosphine)palladium were added. The vessel was closed and the reaction mixture was heated to 120° C. in a CEM Discover microwave reactor for 60 min. The reaction mixture was filtered through a depth filter, which was rinsed with methanol. The reaction was conducted twice in total and the batches were combined prior to purification. The filtrate was concentrated under reduced pressure and the residue was separated by chromatography by MPLC on silica gel (gradient: methanol/dichloromethane 0:100→5:95). Subsequently, the crude product obtained was washed with acetonitrile and dried. This gave 78 mg (98% purity, 38% yield) of 5-(1-methylpyrazol-4-yl)-2-(3-pyridyl)pyrazolo[3,4-b]pyridine.

¹H-NMR (400.0 MHz, d₆-DMSO): δ=9.376(2.66); 9.370(2.64); 9.226(6.44); 9.030(3.40); 9.024(3.48); 8.696(1.78); 8.693(2.00); 8.684(1.85); 8.681(1.94); 8.549(1.04); 8.545(1.24); 8.542(1.23); 8.539(1.10); 8.528(1.13); 8.524(1.26); 8.521(1.31); 8.518(1.08); 8.378(3.51); 8.372(3.46); 8.329(4.70); 8.053(4.82); 7.693(1.44); 7.681(1.43); 7.672(1.39); 7.661(1.32); 3.905(16.00); 3.320(18.85); 2.672(0.33); 2.507(39.14); 2.503(50.50); 2.499(38.59); 2.330(0.33); −0.000 (2.42) ppm.

### EXAMPLE 10: N-[2-(3-PYRIDYL)PYRAZOLO[3,4-B]PYRIDIN-5-YL]N-METHYL-3-METHYLSULPHANYLPROPANAMIDE

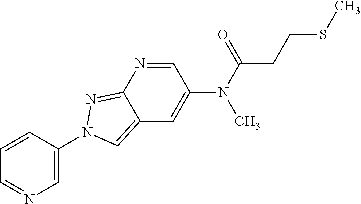 [SMILES: CSCCC(=O)N(C)c1cnc2nn(-c3cccnc3)cc2c1]

To a solution of 23 mg (73 μmol) of 3-methylsulphanyl-N-[2-(3-pyridyppyrazolo[3,4-b]pyridin-5-yl]propanamide (Example 6) in dry N,N-dimethylformamide were added, at 0° C., 4.4 mg (0.11 mmol) of sodium hydride. After 15 min, 15.5 mg (0.11 mmol) of iodomethane were added. The reaction mixture was stirred at room temperature overnight, water was added and then the mixture was extracted repeatedly with ethyl acetate. The combined organic phases were dried with sodium sulphate and filtered and the solvent was removed under reduced pressure. The residue was separated by chromatography by MPLC on silica gel (gradient: ethyl acetate/cyclohexane/methanol 0:100:0→100:0:0→0:0:100). This gave 3.3 mg (80% purity, 11% yield) of N-[2-(3-pyridyl)pyrazolo[3,4-b]pyridin-5-yl]-N-methyl-3-methylsulphanylpropanamide.

¹H-NMR (601.6 MHz, CDCl₃): δ=9.238 (4.2); 9.234 (5.5); 8.744 (2.6); 8.738 (3.4); 8.646 (4.8); 8.642 (4.8); 8.635 (1.7); 8.631 (1.4); 8.562 (5.6); 8.546 (3.1); 8.405 (2.8); 8.403 (2.9); 8.401 (2.6); 8.392 (3.0); 8.390 (3.0); 8.387 (2.7); 7.990 (3.7); 7.987 (3.8); 7.928 (1.5); 7.924 (1.5); 7.569 (1.8); 7.566 (1.6); 7.561 (2.0); 7.557 (2.3); 7.548 (1.9); 7.433 (0.6); 7.262 (103.7); 7.085 (0.6); 6.462 (0.7); 6.460 (0.7); 6.435 (0.9); 6.432 (0.8); 5.614 (0.3); 3.453 (5.4); 3.375 (15.6); 2.802 (2.6); 2.790 (5.2); 2.778 (3.0); 2.573 (0.6); 2.429 (2.8); 2.417 (4.9); 2.405 (2.5); 2.008 (16.0); 1.568 (68.0; 1.427 (0.5); 1.423 (0.7); 1.336 (0.4); 1.333 (0.5); 1.284 (0.8); 1.277 (0.7); 1.254 (3.4); 0.892 (0.4); 0.880 (0.7); 0.868 (0.4); 0.844 (0.4); 0.097 (0.4); 0.069 (1.4); 0.005 (2.9); 0.000 (105.5); −0.006 (4.3); −0.100 (0.5) ppm.

### Synthesis of Intermediates

### Sodium 2-nitropropanediolate hydrate (1:1:1)

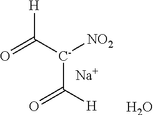

To a stirred solution of 55 g (0.772 mol) of sodium nitrite in 100 ml of ethanol were added gradually and in portions, at a reaction temperature of 55° C., 50 g (0.193 mol) of (2Z)-2,3-dibromo-4-oxo-2-butenoic acid. Thereafter, the reaction mixture was stirred at 55° C. for another 1 hour. After the reaction had ended, the mixture was cooled gradually to room temperature and then to 10° C., in the course of which a solid formed. This solid was filtered off, washed with ethanol and recrystallized from ethanol. This gave 7.0 g (yield: 31% of theory) of sodium 2-nitropropanediolate hydrate (1:1:1) as a brown solid.

In accordance with the description of the preparation processes according to the invention, it was also possible to prepare, for example, the compounds of the formula (I) listed in Table 1 below.

Compounds of the formula (I) and possibly also those not covered by the formula (I) are listed in the table below. Any compounds not covered by the formula (I) also form part of the subject-matter of the invention.

TABLE 1
Compounds of the formula

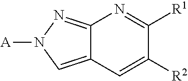

| Com- | | | |
|---|---|---|---|
| pound | | | |
| No. | A1 | R¹ | R² |
| 1 | 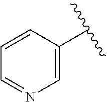 [SMILES: *c1cccnc1] | H | 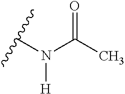 [SMILES: *NC(C)=O] |
| 2 | 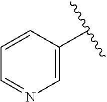 [SMILES: *c1cccnc1] | H | 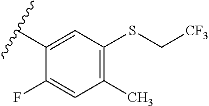 [SMILES: *c1cc(SCC(F)(F)F)c(C)cc1F] |
| 3 | 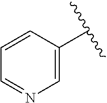 [SMILES: *c1cccnc1] | H | 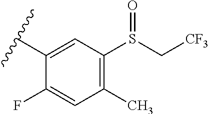 [SMILES: *c1cc(S(=O)CC(F)(F)F)c(C)cc1F] |
| 4 | 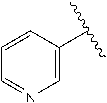 [SMILES: *c1cccnc1] | H | 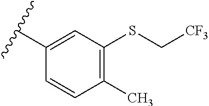 [SMILES: *c1ccc(C)c(SCC(F)(F)F)c1] |
| 5 | 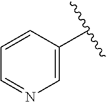 [SMILES: *c1cccnc1] | H | 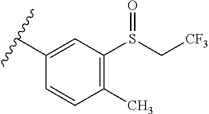 [SMILES: *c1ccc(C)c(S(=O)CC(F)(F)F)c1] |
| 6 | 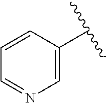 [SMILES: *c1cccnc1] | H | 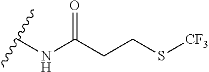 [SMILES: *NC(=O)CCSC(F)(F)F] |
| 7 | 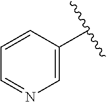 [SMILES: *c1cccnc1] | H | 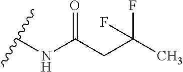 [SMILES: *NC(=O)CC(C)(F)F] |
| 8 | 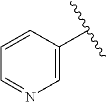 [SMILES: *c1cccnc1] | H | 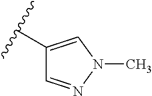 [SMILES: *c1cnn(C)c1] |
| 9 | 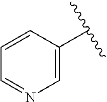 [SMILES: *c1cccnc1] | H | 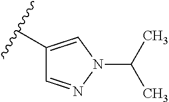 [SMILES: *c1cnn(C(C)C)c1] |
| 10 | 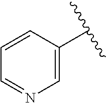 [SMILES: *c1cccnc1] | H | 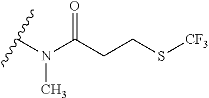 [SMILES: *N(C)C(=O)CCSC(F)(F)F] |

TABLE 2
Analytical data for the compounds 1-8 reported

| Ex. No. | logP[a] | logP[b] | ¹H-NMR [δ (ppm)] or LC-MS [m/z] |
|---|---|---|---|
| 1 | | 0.54 | ¹H NMR(400.0 MHz, d₆-DMSO): δ = 2.12 (s, 3H, CH₃); 7.65-7.68; |
| | | | 8.46-8.49; 8.62-8.68 (4m, 5H, hetaryl); 9.17 (s, 1H, hetaryl); 9.31 |
| | | | (d, 1H; hetaryl); 10.30 (s, 1H, NH). LC-MS (m/z): 254.1 (M + 1) |
| 2 | 3.44 | 3.22 | ¹H-NMR(400.0 MHz, d₆-DMSO): δ = 9.402 (3.4); 9.395 (3.4); 9.369 (9.7); |
| | | | 8.909 (2.2); 8.904 (4.1); 8.899 (2.3); 8.726 (2.3); 8.723 (2.6); 8.714 (2.5); |
| | | | 8.711 (2.6); 8.582 (1.3); 8.579 (1.6); 8.576 (1.5); 8.572 (1.4); 8.561 (1.5); |
| | | | 8.558 (1.5); 8.555 (1.7); 8.551 (1.4); 8.499 (2.7); 8.496 (3.2); 8.494 (3.1); |
| | | | 8.491 (2.7); 7.859 (3.1); 7.840 (3.1); 7.719 (1.9); 7.707 (1.8); 7.698 (1.8); |
| | | | 7.686 (1.7); 7.390 (2.7); 7.361 (2.6); 4.118 (1.3); 4.092 (4.1); 4.066 (4.2); |
| | | | 4.040 (1.5); 3.330 (28.9); 2.677 (0.4); 2.673 (0.5); 2.668 (0.4); 2.526 (1.5); |
| | | | 2.513 (29.3); 2.508 (59.9); 2.504 (79.1); 2.499 (58.1); 2.495 (28.8); 2.458 |
| | | | (16.0); 2.335 (0.4); 2.331 (0.5); 2.326 (0.4); 1.990 (0.5); 0.146 (0.6); 0.008 |
| | | | (4.6); 0.000 (127.8); −0.009 (5.3); −0.150 (0.6). |
| 3 | 2.14 | 2.11 | 1H NMR(400.0 MHz, d6 DMSO): δ = 9.408 (3.5); 9.402 (3.5); 9.389 (9.4); |
| | | | 8.970 (2.2); 8.965 (4.0); 8.960 (2.3); 8.732 (2.4); 8.729 (2.6); 8.720 (2.5); |
| | | | 8.717 (2.6); 8.589 (4.5); 8.585 (4.7); 8.583 (4.2); 8.568 (1.5); 8.565 (1.6); |
| | | | 8.562 (1.7); 8.558 (1.4); 8.318 (0.4); 8.117 (3.2); 8.097 (3.2); 7.724 (1.8); |
| | | | 7.713 (1.8); 7.704 (1.8); 7.693 (1.7); 7.504 (2.5); 7.476 (2.5); 4.335 (0.7); |
| | | | 4.325 (0.5); 4.307 (0.8); 4.298 (1.4); 4.280 (0.4); 4.271 (1.7); 4.243 (1.7); |
| | | | 4.215 (1.5); 4.206 (0.8); 4.188 (0.6); 4.179 (0.7); 4.038 (0.4); 4.020 (0.4); |
| | | | 3.330 (87.7); 2.676 (0.7); 2.672 (0.9); 2.667 (0.7); 2.525 (2.7); 2.512 (54.7); |
| | | | 2.507 (109.0); 2.503 (142.0); 2.498 (103.8); 2.494 (51.5); 2.476 (16.0); 2.334 |
| | | | (0.7); 2.330 (0.9); 2.325 (0.7); 1.989 (1.6); 1.193 (0.4); 1.175 (0.9); 1.158 (0.4); |
| | | | 0.146 (0.7); 0.008 (5.8); 0.000 (150.5); −0.009 (5.9); −0.150 (0.7). |
| 4 | 3.14 | 3.10 | ¹H-NMR(400.0 MHz, d₆-DMSO): δ = 9.409(3.4); 9.403(3.6); 9.342(9.9); |
| | | | 9.330(0.5); 9.106(4.9); 9.100(5.1); 8.719(2.5); 8.715(2.7); 8.707(2.7); |
| | | | 8.703(2.8); 8.586(1.4); 8.583(1.7); 8.579(1.7); 8.576(1.7); 8.570(5.2); |
| | | | 8.565(6.2); 8.559(2.2); 8.555(1.7); 7.925(3.5); 7.921(3.7); 7.714(2.0); |
| | | | 7.702(1.9); 7.693(1.9); 7.682(1.9); 7.680(1.8); 7.657(2.0); 7.652(2.0); |
| | | | 7.637(2.2); 7.632(2.2); 7.416(3.1); 7.396(2.6); 4.228(1.3); 4.202(4.0); |
| | | | 4.176(4.2); 4.150(1.4); 3.333(55.0); 2.678(0.4); 2.673(0.5); 2.669(0.4); |
| | | | 2.526(1.5); 2.521(2.2); 2.513(30.9); 2.508(63.6); 2.504(83.6); 2.499(60.6); |
| | | | 2.495(29.6); 2.420(16.0); 2.369(0.7); 2.335(0.4); 2.331(0.6); 2.326(0.5); |
| | | | 1.990(0.3); 0.008(1.6); 0.000(51.4); −0.009(1.9). |
| 5 | 2.07 | 2.03 | ¹H-NMR(400.0 MHz, d₆-DMSO): δ = 9.413(3.6); 9.406(3.7); |
| | | | 9.361(9.9); 9.144(5.0); 9.138(5.1); 8.725(2.6); 8.722(2.8); 8.713(2.7); 8.710(2.8); |
| | | | 8.646(5.1); 8.640(5.0); 8.591(1.5); 8.587(1.7); 8.584(1.6); 8.580(1.5); 8.570(1.6); |
| | | | 8.566(1.7); 8.563(1.8); 8.560(1.5); 8.241(4.0); 8.236(4.2); 7.983(2.0); 7.978(1.9); |
| | | | 7.963(2.2); 7.958(2.2); 7.720(2.0); 7.708(1.9); 7.699(2.0); 7.687(1.9); 7.527(3.0); |
| | | | 7.507(2.7); 5.759(0.4); 4.303(0.6); 4.294(0.5); 4.276(0.8); 4.266(1.6); 4.249(0.7); |
| | | | 4.239(1.6); 4.222(1.5); 4.212(0.8); 4.195(1.7); 4.186(0.8); 4.167(0.6); 4.158(0.7); |
| | | | 3.333(27.3); 2.677(0.3); 2.673(0.4); 2.668(0.3); 2.526(1.0); 2.513(24.1); 2.509(49.3); |
| | | | 2.504(64.7); 2.499(47.5); 2.495(23.7); 2.460(16.0); 2.331(0.4); 0.146(0.5); 0.008(4.0); |
| | | | 0.000(98.7); −0.009(4.5); −0.015(0.7); −0.018(0.7); −0.021(0.6); −0.150(0.4). |
| 6 | 1.15 | 1.21 | ¹H-NMR(400.0 MHz, d₆-DMSO): δ = 10.355(2.1); 9.330(2.1); 9.323(2.1); |
| | | | 9.192(5.3); 8.685(1.5); 8.682(1.5); 8.673(1.7); 8.670(2.0); 8.660(5.0); |
| | | | 8.657(4.8); 8.651(1.1); 8.503(0.8); 8.500(0.9); 8.497(0.9); 8.493(0.8); |
| | | | 8.482(0.9); 8.479(1.0); 8.476(1.0); 8.472(0.8); 7.686(1.1); 7.674(1.1); |
| | | | 7.665(1.1); 7.653(1.1); 3.506(0.3); 3.378(3.0); 2.813(1.0); 2.796(2.9); |
| | | | 2.779(2.2); 2.712(2.1); 2.695(2.7); 2.677(1.3); 2.508(50.0); 2.503(63.8); |
| | | | 2.499(46.2); 2.330(0.4); 2.116(16.0); 1.990(0.4); 1.298(0.4); 1.259(0.6); |
| | | | 1.232(0.7); 0.146(0.4); 0.008(3.3); 0.000(81.7); −0.008(3.3); −0.150(0.4). |
| 7 | 1.22 | 1.22 | ¹H-NMR(400.0 MHz, d₆-DMSO): δ = 10.530(4.6); 9.332(4.7); 9.326(4.7); |
| | | | 9.202(12.5); 9.192(0.5); 8.690(3.3); 8.686(3.7); 8.678(3.5); 8.675(3.7); |
| | | | 8.659(2.4); 8.651(16.0); 8.507(1.9); 8.503(2.2); 8.500(2.2); 8.497(2.0); |
| | | | 8.486(2.0); 8.482(2.3); 8.479(2.4); 8.476(2.0); 8.314(0.8); 7.686(2.6); |
| | | | 7.674(2.5); 7.665(2.4); 7.655(2.3); 7.653(2.3); 3.320(135.0); 3.171(3.1); |
| | | | 3.133(6.5); 3.096(3.3); 2.676(1.0); 2.671(1.3); 2.667(1.0); 2.524(4.2); |
| | | | 2.511(69.5); 2.507(137.1); 2.502(180.3); 2.498(136.2); 2.493(70.4); |
| | | | 2.333(0.8); 2.329(1.1); 2.325(0.8); 1.861(5.5); 1.813(11.6); 1.765(5.9); |
| | | | 1.234(0.8); 0.146(0.6); 0.008(5.9); 0.000(133.4); −0.009(5.6); −0.150(0.5). |
| 8 | 1.00 | 1.15 | ¹H-NMR(400.0 MHz, d₆-DMSO): □ □ = 9.376(2.66); 9.370(2.64); |
| | | | 9.226(6.44); 9.030(3.40); 9.024(3.48); 8.696(1.78); 8.693(2.00); 8.684(1.85); |
| | | | 8.681(1.94); 8.549(1.04); 8.545(1.24); 8.542(1.23); 8.539(1.10); 8.528(1.13); |
| | | | 8.524(1.26); 8.521(1.31); 8.518(1.08); 8.378(3.51); 8.372(3.46); 8.329(4.70); |
| | | | 8.053(4.82); 7.693(1.44); 7.681(1.43); 7.672(1.39); 7.661(1.32); 3.905(16.00); |
| | | | 3.320(18.85); 2.672(0.33); 2.507(39.14); 2.503(50.50); 2.499(38.59); |
| | | | 2.330(0.33); −0.000 (2.42). |
| 9 | 1.54 | 1.53 | ¹H-NMR(400.0 MHz, d₆-DMSO): δ = 9.379(2.3); 9.372(2.4); |
| | | | 9.223(6.0); 9.061(3.1); 9.055(3.2); 8.696(1.6), 8.693(1.7); 8.684(1.7); |
| | | | 8.681(1.7); 8.550(0.9); 8.547(1.1); 8.544(1.1); 8.540(1.0); 8.529(1.0); |
| | | | 8.526(1.1); 8.523(1.2); 8.519(1.0); 8.441(4.3); 8.395(3.2); 8.390(3.2); |
| | | | 8.060(4.4); 7.694(1.3); 7.682(1.3); 7.673(1.3); 7.661(1.2); 4.571(0.4); |
| | | | 4.554(1.1); 4.537(1.5); 4.521(1.1); 4.504(0.4); 3.321(35.5); 2.672(0.3); |
| | | | 2.507(36.2); 2.503(47.6); 2.498(36.1); 1.989(0.4); 1.489(16.0); |
| | | | 1.472(15.9); 0.008(2.0); 0.000(47.9); −0.008(2.8) |
| 10 | 1.16 | 1.20 | ¹H-NMR(601.6 MHz, CDCl₃): δ = 9.238(4.2); 9.234(5.5); |
| | | | 8.744(2.6); 8.738(3.4); 8.646(4.8); 8.642(4.8); 8.635(1.7); 8.631(1.4); |
| | | | 8.562(5.6); 8.546(3.1); 8.405(2.8); 8.403(2.9); 8.401(2.6); 8.392(3.0); |
| | | | 8.390(3.0); 8.387(2.7); 7.990(3.7); 7.987(3.8); 7.928(1.5); 7.924(1.5); |
| | | | 7.569(1.8); 7.566(1.6); 7.561(2.0); 7.557(2.3); 7.548(1.9); 7.433(0.6); |
| | | | 7.262(103.7); 7.085(0.6); 6.462(0.7); 6.460(0.7); 6.435(0.9); 6.432(0.8); |
| | | | 5.614(0.3); 3.453(5.4); 3.375(15.6); 2.802(2.6); 2.790(5.2); 2.778(3.0); |
| | | | 2.573(0.6); 2.429(2.8); 2.417(4.9); 2.405(2.5); 2.008(16.0); 1.568(68.1); |
| | | | 1.427(0.5); 1.423(0.7); 1.336(0.4); 1.333(0.5); 1.284(0.8); 1.277(0.7); |
| | | | 1.254(3.4); 0.892(0.4); 0.880(0.7); 0.868(0.4); 0.844(0.4); 0.097(0.4); |
| | | | 0.069(1.4); 0.005(2.9); 0.000(105.5); −0.006(4.3); −0.100(0.5) |

### Biological Examples

Myzus persicae—Spray Test
Solvent: 78 parts by weight of acetone
   1.5 parts by weight of dimethylformamide
Emulsifier: alkylaryl polyglycol ether

To produce a suitable active ingredient formulation, 1 part by weight of active ingredient is dissolved with the specified parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) infested by all stages of the green peach aphid (Myzus persicae) are sprayed with an active ingredient formulation of the desired concentration.

After 6 days, the efficacy in % is determined. 100% means that all the aphids have been killed and 0% means that none of the aphids have been killed.

In this test, for example, the following compounds from the preparation examples show an efficacy of 100% at an application rate of 500 g/ha: 4, 5, 6

In this test, for example, the following compounds from the preparation examples show an efficacy of 90% at an application rate of 500 g/ha: 2, 3, 7, 8, 9, 10

Aphis gossypii—Spray Test
Solvent: 7 parts by weight of dimethylformamide
Emulsifier: alkylaryl polyglycol ether

To produce a suitable active ingredient formulation, 1 part by weight of active ingredient is dissolved with the specified parts by weight of solvent and made up with water containing an emulsifier concentration of 1000 ppm until the desired concentration is attained. To produce further test concentrations, the formulation is diluted with emulsifier-containing water. If the addition of ammonium salts or/and penetrants is required, these are each added in a concentration of 1000 ppm to the formulation solution.

Cotton plants (Gossypium hirsutum) heavily infested by the cotton aphid (Aphis gossypii) are sprayed with an active ingredient formulation of the desired concentration.

After 6 days, the kill in % is determined. 100% means that all the aphids have been killed and 0% means that none of the aphids have been killed.

In this test, for example, the following compound from the preparation examples shows an efficacy of 95% at an application rate of 4 ppm: 1

1. Compound of formula (I) 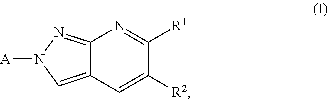
in which
A is a radical from the group of (A-a) to (A-f) 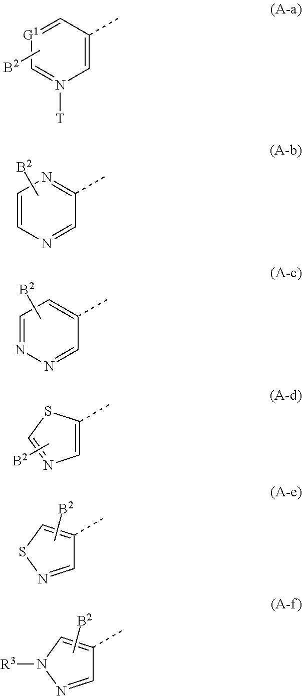
in which the broken line denotes the bond to the nitrogen atom of the bicyclic system of the formula (I) and
G¹ is N or C—B¹,
B¹ is a radical from the group of hydrogen, halogen, cyano, nitro, alkyl, haloalkyl, alkoxy, haloalkoxy and in each case optionally substituted cycloalkyl and cycloalkenyl,
B² is a radical from the group of hydrogen, halogen, cyano, nitro, alkyl, haloalkyl, alkoxy, haloalkoxy and in each case optionally substituted cycloalkyl and cycloalkenyl,
T is oxygen or an electron pair,
R¹ is a radical from the group of hydrogen, alkyl, alkoxy and cyano,
R² a) is a B radical from the group of (B-1) to (B-36) 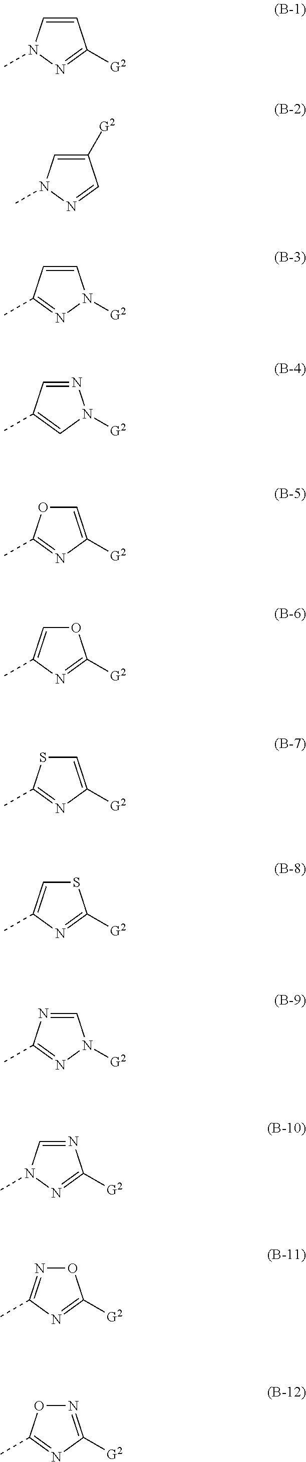 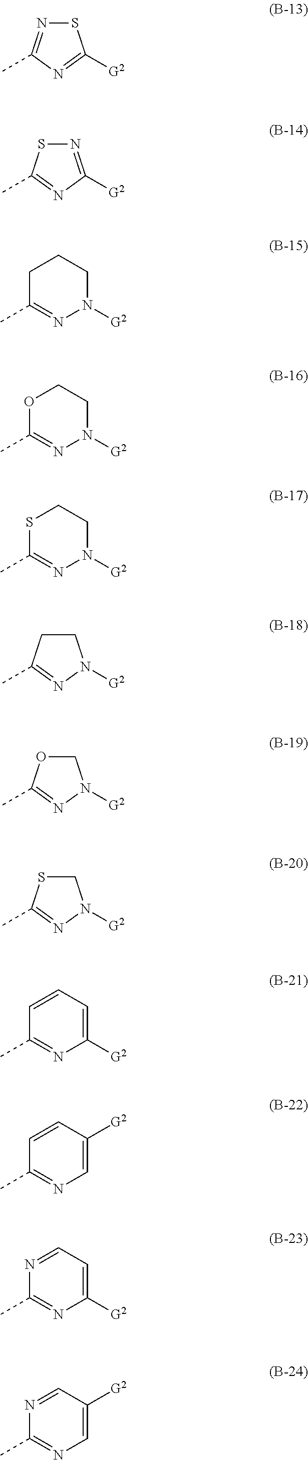 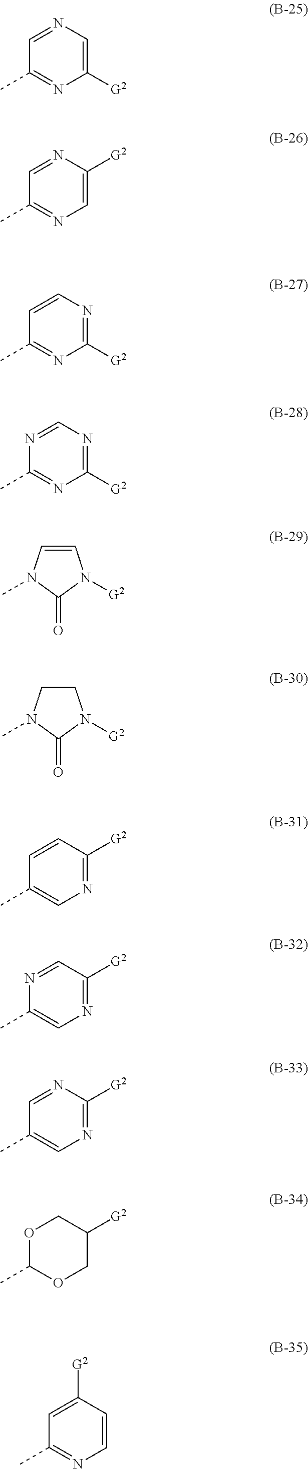 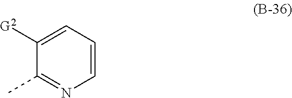
in which the broken line denotes the bond to the carbon atom of the bicyclic system of the formula (I), or
R² b) is a D radical from the group of (D-1) to (D-3) 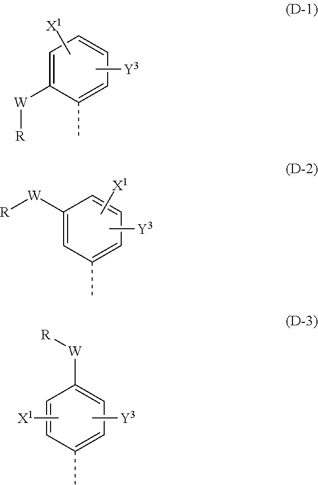
in which the broken line denotes the bond to the carbon atom of the bicyclic system of the formula (I), or
R² c) is a radical of the formula 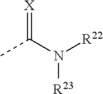
in which the broken line denotes the bond to the carbon atom of the bicyclic system of the formula (I), or
R² d) is a radical of the formula 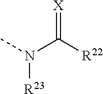
in which the broken line denotes the bond to the carbon atom of the bicyclic system of the formula (I), or
R² e) is an F radical from the group of (F-1) to (F-11) 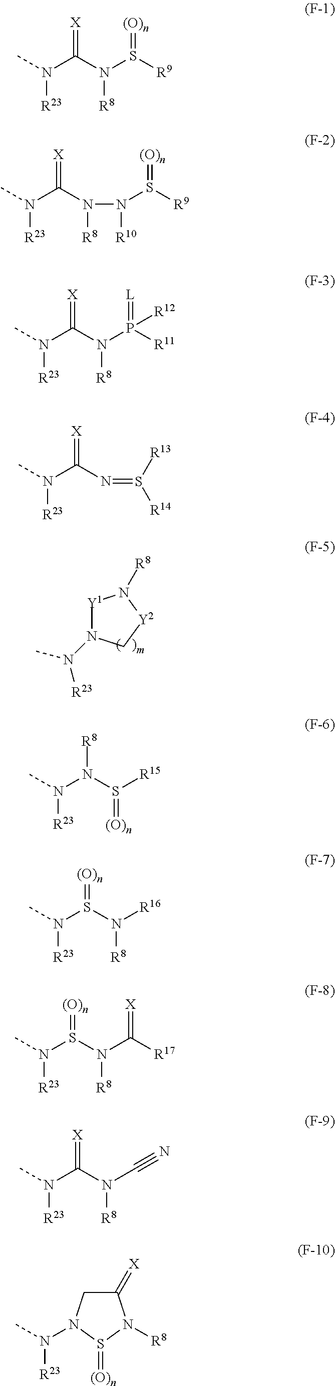 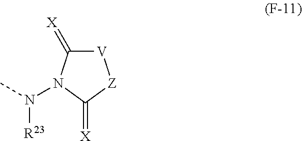
in which the broken line denotes the bond to the carbon atom of the bicyclic system of the formula (I), or
R² f) is a radical from the group of haloalkyl, carboxyl and amino,
in which
G² is hydrogen or a radical from the group of halogen, nitro, amino, cyano, alkylamino, haloalkylamino, dialkylamino, alkyl, haloalkyl, alkoxycarbonylalkyl, saturated or unsaturated cycloalkyl which is optionally substituted and optionally interrupted by one or more heteroatoms, cycloalkylalkyl, alkoxy, haloalkoxy, alkoxyalkyl, halogenated alkoxyalkyl, alkylthioalkyl, alkylsulphinylalkyl, alkylsulphonylalkyl, bis(alkoxy)alkyl, bis(haloalkoxy)alkyl, alkoxy(alkylsulphanyl)alkyl, alkoxy (alkyl sulphinyl)alkyl, alkoxy(alkylsulphonyl)alkyl, bis(alkyl sulphanyl)alkyl, bis(haloalkylsulphanyl)alkyl, bis(hydroxyalkylsulphanyl)alkyl, alkoxycarbonyl, alkoxycarbonylalkyl, alpha-hydroxyiminoalkoxycarbonylalkyl, alpha-alkoxyiminoalkoxycarbonylalkyl, C(X²)NR³R⁴, NR⁶R⁷, alkylthio, alkylsulphinyl, alkylsulphonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may in turn be substituted by alkyl, haloalkyl, alkoxy and alkoxyalkyl), phenyl (which for its part may in turn be substituted by halogen, cyano, nitro, alkyl and haloalkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may in turn be substituted by halogen, nitro, alkyl, haloalkyl, alkoxy, haloalkoxy, alkoxyalkyl, alkylthio, alkylthioalkyl and cycloalkyl) and the heteroarylalkyl radicals triazolylalkyl, pyridylalkyl, pyrimidylalkyl and oxadiazolylalkyl (which for their part may in turn be substituted by halogen and alkyl),
or
G² is a radical from the group of (C-1) to (C-9) 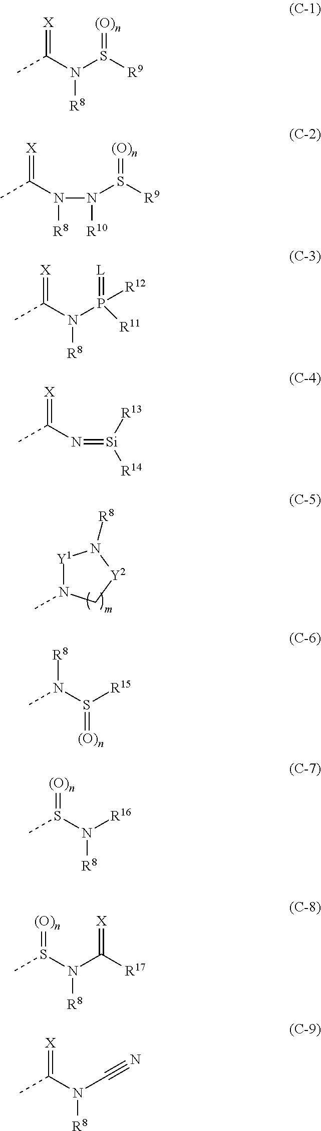
in which the broken line denotes the bond to the radicals (B-1) to (B-34),
X is oxygen or sulphur,
X¹ is a radical from the group of hydrogen, halogen, cyano, nitro, alkyl, haloalkyl, cycloalkyl, alkoxy and haloalkoxy,
X² is oxygen, sulphur, NR⁵ or NOH,
L is oxygen or sulphur,
V—Z is R²⁴CH—CHR²⁵ or R²⁴C═CR²⁵,
n is 1 or 2,
m is 1, 2, 3 or 4,
R is NR¹⁸R¹⁹, or is an in each case optionally substituted radical from the group of alkyl, alkenyl, alkynyl, alkoxyalkyl, alkyl-S-alkyl, alkyl-S(O)-alkyl, alkyl-S(O)₂-alkyl, R¹⁸—CO-alkyl, NR¹⁸R¹⁹—CO-alkyl, cycloalkyl, cycloalkenyl, cycloalkylalkyl, cycloalkenylalkyl, heterocyclyl, heterocyclylalkyl, phenyl, phenylalkyl, hetaryl and hetarylalkyl,
R³ is hydrogen or alkyl,
R⁴ is a radical from the group of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, alkylsulphinylalkyl, alkylsulphonylalkyl, aryl, arylalkyl and hetarylalkyl,
R⁵ is a radical from the group of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, aryl, arylalkyl and hetarylalkyl, or
R³ and R⁴ together with the nitrogen atom to which they are bonded form a ring which may contain one or more further heteroatoms from the group of nitrogen, oxygen and sulphur, or
R³ and R⁵ together with the nitrogen atoms to which they are bonded form a ring,
R⁶ is hydrogen or alkyl,
R⁷ is a radical from the group of hydrogen, alkyl, haloalkyl, cyanoalkyl, alkynyl, cycloalkyl, cycloalkylalkyl, alkoxy, haloalkoxy, alkoxyalkyl, alkylthioalkyl, alkylsulphinylalkyl, alkylsulphonylalkyl, alkoxycarbonyl, alkoxycarbonylalkyl, alkylthioalkyl, aryl, arylalkyl and hetarylalkyl, or
R⁶ and R⁷ together with the nitrogen atom to which they are bonded form a ring which may contain one or more further heteroatoms from the group of nitrogen, oxygen and sulphur,
R⁸ is a radical from the group of hydrogen, alkyl, haloalkyl, cyanoalkyl, alkoxy, haloalkoxy, alkenyl, alkoxyalkyl, in each case optionally halogen-substituted alkylcarbonyl and alkylsulphonyl, optionally halogen-substituted alkoxycarbonyl, optionally halogen-, alkyl-, alkoxy-, haloalkyl- and cyano-substituted cycloalkylcarbonyl, or is a cation, or an optionally alkyl- or arylalkyl-substituted ammonium ion,
R⁹ is a radical from the group of in each case optionally substituted alkyl, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R⁹ in the (C-1) and (F-1) radicals, together with the N—S(O)n group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁰ is hydrogen or alkyl,
R⁸ and R¹⁰ in the (C-2) and (F-2) radicals, together with the nitrogen atoms to which they are bonded, may also be a saturated or unsaturated and optionally substituted 4- to 8-membered ring which may contain at least one further heteroatom from the group of sulphur, oxygen (where oxygen atoms must not be directly adjacent to one another) and nitrogen and/or at least one carbonyl group,
R⁹ and R¹⁰ in the (C-2) and (F-2) radicals, together with the N—S(O)n group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹¹ is an in each case optionally substituted radical from the group of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹² is an in each case optionally substituted radical from the group of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹¹ and R¹² in the (C-3) and (F-3) radicals, together with the phosphorus atom to which they are bonded, may also form a saturated or unsaturated and optionally substituted 5- to 7-membered ring which may contain one or two heteroatoms from the group of oxygen (where oxygen atoms must not be directly adjacent to one another) and sulphur,
R¹³ is an in each case optionally substituted radical from the group of alkyl, alkenyl, alkynyl, phenyl and phenylalkyl,
R¹⁴ is an in each case optionally substituted radical from the group of alkyl, alkenyl, alkynyl, phenyl and phenylalkyl,
R¹⁵ is a radical from the group of in each case optionally substituted alkyl, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁵ in the (C-6) and (F-6) radicals, together with the N—S(O)n group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁶ is a radical from the group of hydrogen, in each case optionally substituted alkyl, alkoxy, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁶ in the (C-7) and (F-7) radicals, together with the nitrogen atom to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁷ is a radical from the group of in each case optionally substituted alkyl, alkoxy, alkenyl and alkynyl, in each case optionally substituted cycloalkyl, cycloalkylalkyl and cycloalkenyl, in which the rings may contain at least one heteroatom from the group of sulphur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁷ in the (C-8) and (F-8) radicals, together with the N—C(X) group to which they are bonded, may also form a saturated or unsaturated and optionally substituted 4- to 8-membered ring which may contain one or more further heteroatoms from the group of sulphur, oxygen (where oxygen atoms must not be directly adjacent to one another) and nitrogen and/or at least one carbonyl group,
R¹⁸ is a radical from the group of hydrogen, hydroxy, in each case optionally substituted alkyl, alkoxy, alkoxyalkyl, alkylthioalkyl, alkylsulphinylalkyl, alkylsulphonylalkyl, alkylcarbonyl, alkoxycarbonyl, alkenyl and alkynyl, in each case optionally substituted cycloalkyl, cycloalkylalkyl, cycloalkenyl and cycloalkenylalkyl, in which the rings may contain at least one heteroatom from the group of sulphur, oxygen (where oxygen atoms must not be directly adjacent to one another) and nitrogen, in each case optionally substituted aryl, arylalkyl, heteroaryl and heteroarylalkyl and an optionally substituted amino group,
R¹⁹ is a radical from the group of hydrogen, is an alkali metal or alkaline earth metal ion or is an ammonium ion optionally mono- to tetrasubstituted by C₁-C₄-alkyl or is an in each case optionally halogen- or cyano-substituted alkyl, alkoxy, alkoxyalkyl, alkylthioalkyl, alkylsulphinylalkyl, alkylsulphonylalkyl radical,
Y¹ and Y² are independently C═O or S(O)₂,
Y³ is a radical from the group of hydrogen, halogen, cyano, alkyl, cycloalkyl, haloalkyl, alkoxy, haloalkoxy and NR²⁰R²¹,
W is a radical from the group of O, S, SO and SO₂,
R²² is a radical from the group of alkyl, optionally halogen-, carbamoyl-, thiocarbamoyl- or cyano-substituted cycloalkyl, haloalkyl, alkoxy, haloalkoxy, alkoxyalkyloxy, alkylthio, alkylsulphinyl, alkylsulphonyl, haloalkylthio, haloalkylsulphinyl, haloalkylsulphonyl, alkylthioalkyl, alkylsulphinylalkyl, alkylsulphonylalkyl, alkyl thioalkyloxy, alkyl sulphinylalkyloxy, alkylsulphonylalkyloxy, haloalkylthioalkyl, haloalkylsulphinylalkyl, haloalkylsulphonylalkyl, alkyl thioalkenyl, alkyl sulphinylalkenyl, alkyl sulphonylalkenyl, alkenylthioalkyl, alkenylsulphinylalkyl, alkenylsulphonylalkyl, alkylcarbonylalkyl, haloalkylcarbonylalkyl, alkoxyalkyl, haloalkoxyalkyl, alkoxycarbonylalkyl, haloalkoxycarbonylalkyl, alkylaminosulphonyl, di(alkylamino)sulphonyl, or
in the case that R²=d)
R²² is also optionally substituted aryl or a radical from the group of E-1 to E-51 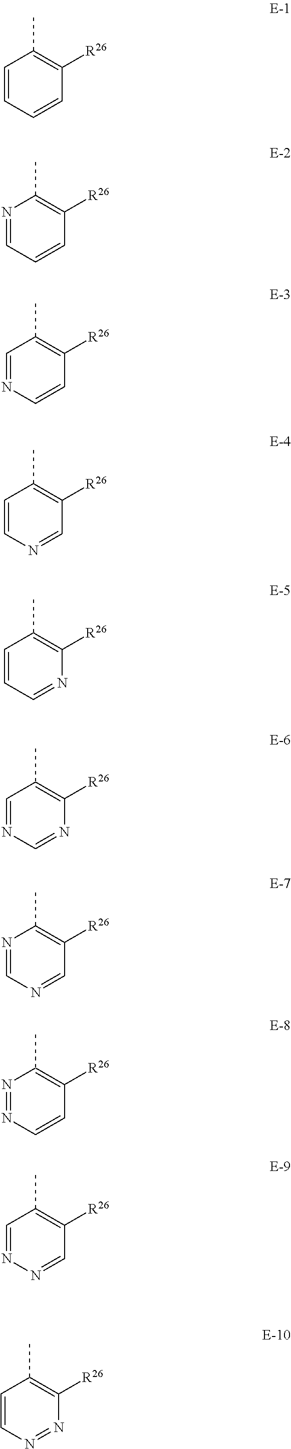 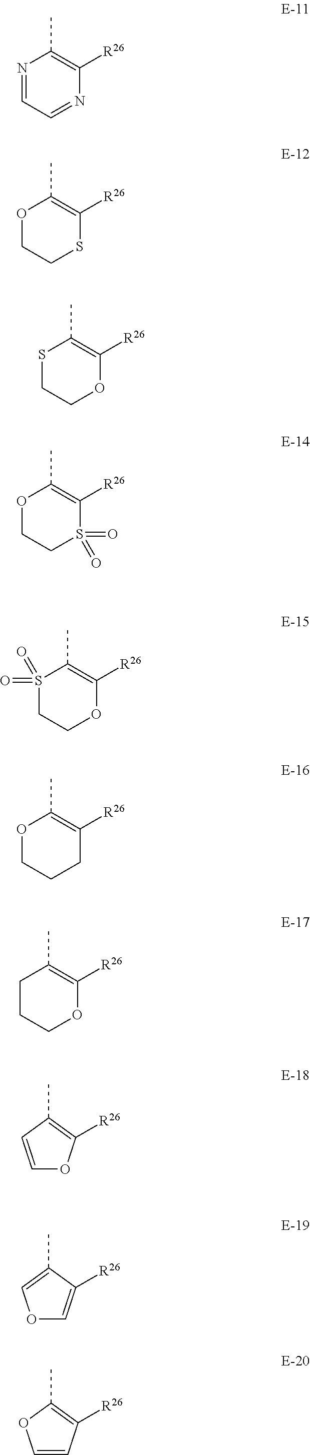 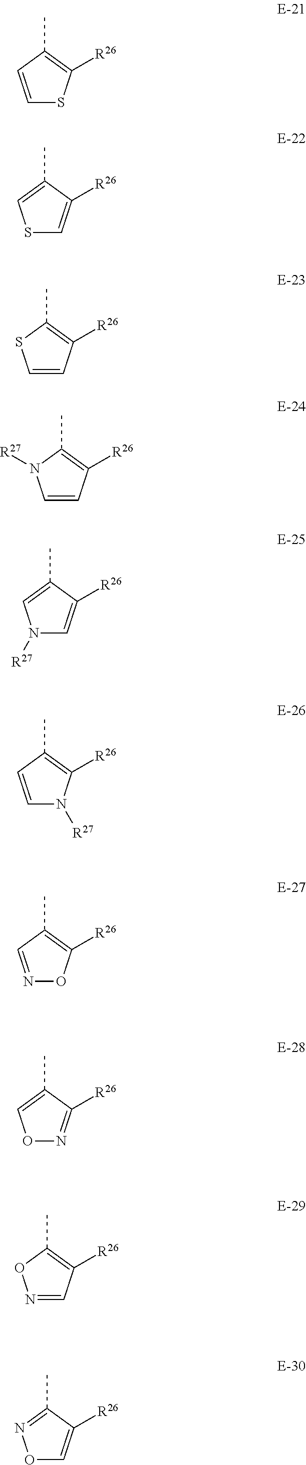 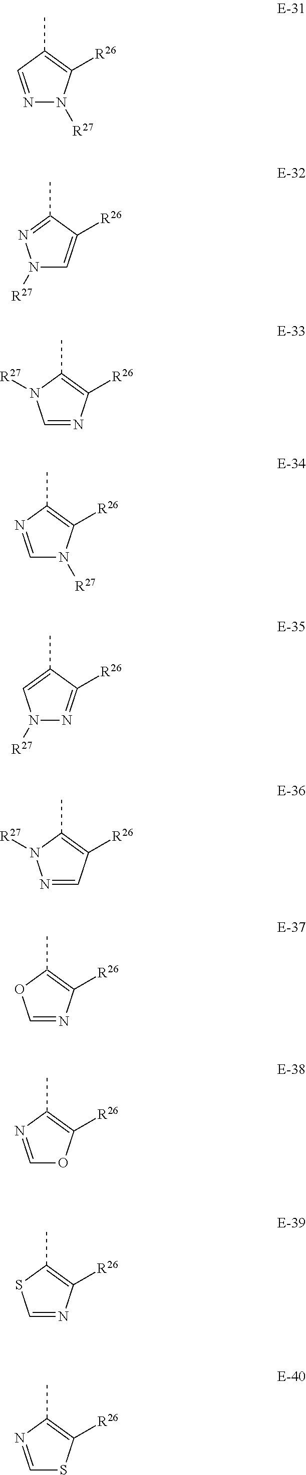 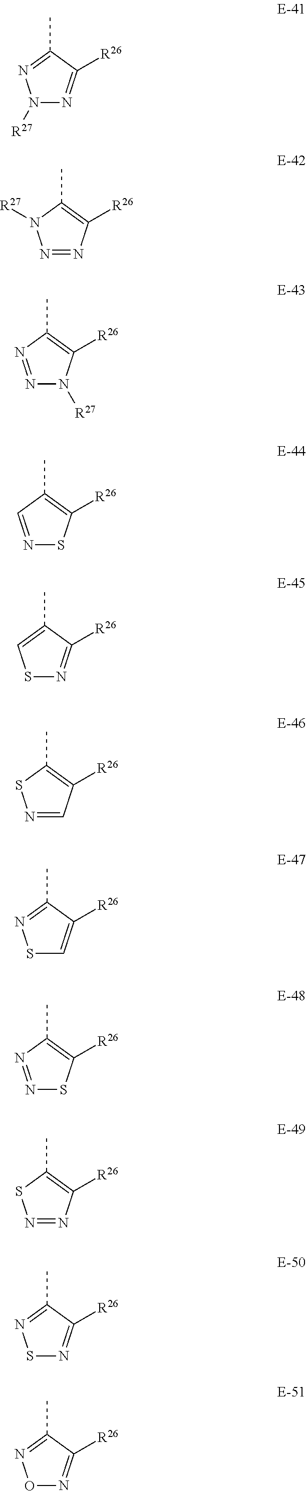
R²⁰ is a radical from the group of hydrogen, halogen, cyano, nitro, amino, hydroxy and in each case optionally substituted alkyl, alkenyl, alkynyl, cycloalkyl, cycloalkylalkyl, alkoxy, alkenyloxy, alkynyloxy, cycloalkyloxy, alkylcarbonyloxy, alkenylcarbonyloxy, alkynylcarbonyloxy, cycloalkylcarbonyloxy, alkoxycarbonyloxy, alkylsulphonyloxy, alkylamino, alkenylamino, alkynylamino, cycloalkylamino, alkylthio, haloalkylthio, alkenylthio, alkynylthio, cycloalkylthio, alkylsulphinyl, alkylsulphonyl, alkylcarbonyl, alkoxyiminoalkyl, alkoxycarbonyl, aminocarbonyl, alkylaminocarbonyl, dialkylaminocarbonyl, aminothiocarbonyl, alkylaminosulphonyl, alkylsulphonylamino, alkylcarbonylamino, alkenylcarbonylamino, alkynylcarbonylamino, cycloalkylcarbonylamino, alkoxycarbonylamino, alkylthiocarbonylamino, bicycloalkyl, aryl, aryloxy, heteroaryl and heteroaryloxy, where the substituents are independently of one another selected from halogen, cyano, nitro, hydroxy, amino, alkyl and haloalkyl,
R²¹ is a radical from the group of hydrogen, alkyl, cycloalkyl, haloalkyl, alkenyl, alkynyl, cycloalkylalkyl, cyanoalkyl, alkylcarbonyl, alkenylcarbonyl, haloalkylcarbonyl, haloalkenylcarbonyl, alkoxyalkyl, alkoxycarbonyl, alkylsulphonyl and haloalkylsulphonyl,
R²³ is a radical from the group of hydrogen, alkyl, alkenyl, alkynyl, cycloalkyl, cycloalkenyl, alkoxy, alkenyloxy, alkynyloxy, cycloalkyloxy, alkylthioalkyl, alkenylthioalkyl, cyanoalkyl, alkoxyalkyl and
R²⁴ is hydrogen or an in each case optionally substituted radical from the group of alkyl, alkenyl, alkynyl, phenyl and phenylalkyl and
R²⁵ is hydrogen or an in each case optionally substituted radical from the group of alkyl, alkenyl, alkynyl, phenyl and phenylalkyl,
R²⁷ is hydrogen or alkyl and
R²⁶ is a radical from the group of hydrogen, alkyl, haloalkyl, alkenyl, alkynyl, cycloalkyl, cycloalkylalkyl, alkoxyalkyl, alkylthioalkyl, alkylsulphinylalkyl, alkylsulphonylalkyl and cyanoalkyl.

2. Compound of the formula (I) according to claim 1, in which
A is the radical 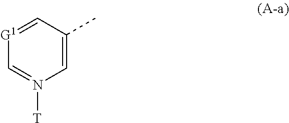
in which the broken line denotes the bond to the nitrogen atom of the bicyclic system of the formula (I),
G¹ is C—B¹,
B¹ is hydrogen,
T is an electron pair,
R¹ is hydrogen,
R² a) is 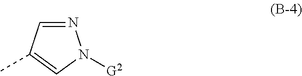
in which the broken line denotes the bond to the carbon atom of the bicyclic system of the formula (I), or
R² b) is the (D-2) radical 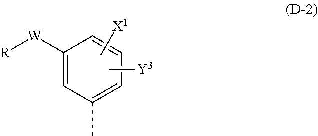
in which the broken line denotes the bond to the carbon atom of the bicyclic system of the formula (I), or
R² c) is the radical of the formula 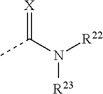
in which the broken line denotes the bond to the carbon atom of the bicyclic system of the formula (I), or
G² is a radical from the group of hydrogen and C₁-C₄-alkyl,
X is oxygen,
X¹ is a radical from the group of hydrogen, fluorine, chlorine and bromine,
R is optionally mono-, di-, tri-, tetra- or penta-fluorine- or -chlorine-substituted C₁-C₄-alkyl,
W is a radical from the group of S, SO and SO₂,
Y³ is methyl or ethyl,
R²² is a radical from the group of C₁-C₆-alkyl, C₁-C₆-haloalkyl and C₁-C₄-alkylthio-C₁-C₄-alkyl and
R²³ is hydrogen or C₁-C₆-alkyl.

3. Composition, comprising a content of at least one compound of formula (I) according to claim 1 and one or more customary extenders and/or surfactants.

4. A compound of the formula (I) according claim 1 for controlling pests.

5. Compound of the formula

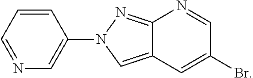 [SMILES: Brc1cnc2nn(-c3cccnc3)cc2c1]

6. Compound of the formula 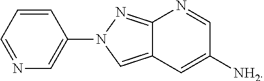 [SMILES: Nc1cnc2nn(-c3cccnc3)cc2c1]
